(19) United States
(12) Patent Application Publication
(10) Pub. No.: US 2023/0372253 A1
(43) Pub. Date: Nov. 23, 2023

(54) NOVEL LIPID-BASED SMALL MOLECULE INTEGRIN RECEPTOR-LIGAND AGONIST ADJUVANTS CARRIER COMPOSITIONS, INTEGRIN AGONIST ADJUVANT PHARMACEUTICAL COMPOSITIONS THEREFROM, AND METHODS FOR MAKING AND USING SAME

(71) Applicant: 7 Hills Pharma LLC, Houston, TX (US)

(72) Inventor: Upendra K. Marathi, III, Houston, TX (US)

(73) Assignee: 7 Hills Pharma LLC, Houston, TX (US)

(21) Appl. No.: 18/142,459

(22) Filed: May 2, 2023

Related U.S. Application Data
(60) Provisional application No. 63/337,960, filed on May 3, 2022.
(60) Provisional application No. 63/348,852, filed on Jun. 3, 2022.

(51) Int. Cl. A61K 9/48; A61K 31/381; A61K 47/44; A61K 47/10; A61K 47/14; A61K 47/24; A61K 47/12

(52) U.S. Cl. CPC A61K 9/4858; A61K 31/381; A61K 47/44; A61K 47/10; A61K 47/14; A61K 47/24; A61K 47/12

(57) ABSTRACT

A pharmaceutical composition comprises a drug-carrier system having a small-molecule drug of low water solubility such as one or more small molecule integrin agonist adjuvants in a substantially non-aqueous carrier composition that comprises at least one phospholipid, at least one neutral lipid, and at least one pharmaceutically acceptable solubilizing agent. The drug-carrier system, when mixed with an aqueous phase, typically forms a non-gelling, substantially non-transparent liquid dispersion. The composition is suitable for administration by a suitable route, e.g., orally, to a subject in need thereof.

### RELATED APPLICATIONS

This application claims priority to and the benefit of U.S. Patent Provisional Patent Application Ser. No. 63/348,852 filed Jun. 3, 2022 (3 Jun. 2022) and 63/337,960 filed May 3, 2022 (3 May 2022).

United States patent applications and patents are incorporated by reference via the closing paragraph: U.S. patent application Ser. No. 14/621,112 filed Feb. 12, 2015; Ser. No. 14/754,931 filed Jun. 30, 2015; Ser. No. 16/516,978 filed Jul. 19, 2019; and Ser. No. 17/471,922 filed Sep. 10, 2021; United States Published Application Nos. 20150250883; 20160000755; 20160000755; and 20200054600; and U.S. Pat. No. 10,342,866 issued Jul. 9, 2019; U.S. Pat. No. 10,709,781 issued Jul. 13, 2020; U.S. Pat. No. 10,716,849 issued Jul. 20, 2020; U.S. Pat. No. 10,709,780 issued Jul. 13, 2020, and U.S. Pat. No. 11,311,619 issued Apr. 26, 2022.

### BACKGROUND OF THE INVENTION

### 1. Field of the Invention

Embodiments of the present invention relate to carrier compositions for poorly water-soluble pharmaceutical agents, poorly water-soluble pharmaceutical agent compositions derived therefrom, and methods for making and using same.

More particularly, embodiments of the present invention relate to carrier compositions for poorly water-soluble, small molecule integrin agonist adjuvant pharmaceutical agents, poorly water-soluble, small molecule integrin agonist adjuvant pharmaceutical agent compositions therefrom, and methods for making and using same, wherein the integrin agonist adjuvant carrier compositions comprising one or more zwitterionic surfactants, one or more non-ionic surfactants, one or more neutral lipids, and one or more polar organic co-solvents or solubilizing agents.

### 2. Description of the Related Art

Integrin agonists are agents that are capable of increasing integrin activation and facilitating interactions between cells possessing integrins and cells possessing integrin associated ligands. These interactions have been found to be important in a wide variety of pharmaceutical applications.

While numerous pharmaceutical carriers are known in the art, there is still a need in the art for carrier compositions for increasing the oral bioavailability of integrin agonists for use in enhancing vaccine efficacies, antigen presentation efficacies, immune response efficacies, and other therapies that involve integrin activation.

### SUMMARY OF THE INVENTION

Embodiments of this disclosure provide carrier compositions for administering poorly water-soluble pharmaceutical agents in the form of lipid-based delivery system comprising of one or more zwitterionic surfactants such as one or more phospholipids, one or more neutral lipids, and one or more co-solvents or solubilizing agents. The lipid-based delivery system may be used to formulate oral poorly water-soluble pharmaceutical agent solutions capable of being encapsulated in or filled in soft gel capsules or hard gel capsules, and when mixed with an aqueous phase, typically form a non-gelling, substantially non-transparent liquid dispersion or emulsion. In certain embodiments, the one or more neutral lipids comprising neutral lipids or phospholipid containing fatty acid groups having more than fourteen carbon atoms (i.e., >C₁₄ fatty acids), which are believed to enhance partitioning of the compositions into the lymphatic circulation and decrease the extent of gastrointestinal mucosal and hepatic CYP450 enzymes. In doing so, the lipid-based drug delivery system will increase solubility and oral bioavailability of poorly soluble integrin agonists with Log P values greater than 5 (i.e., Log P>5).

Embodiments of this disclosure provide integrin agonist carrier compositions comprising one or more zwitterionic surfactants, one or more neutral lipids, and one or more organic solvents.

Embodiments of this disclosure provide integrin agonist pharmaceutical compositions comprising an integrin agonist carrier composition and an effective amount of one or more integrin agonists capable of enhancing cell-to-cell interactions between cell possessing surface integrins and cells possessing surface associated ligands. In certain embodiments, the surface integrins targeted by these agonists including, without limitation, α4β1, α4β7, α5β1, αLβ2 and/or αVβ3 facilitating interactions with ligands including, but are not limited to, VCAM-1, fibronectin, MAdCAM-1, ICAM-1, ICAM-2, and/or vitronectin. In certain embodiments, the integrin agonists are small molecule integrin agonist having molecular weights between about 300 g/mole and about 2000 g/mole, between about 300 g/mole and 1,500 g/mole, or between about 300 g/mole and 1,000 g/mole. In other embodiments, the integrin agonists containing linkers including at least one proponatable group, at least one aromatic or non-aromatic cyclic groups ring, at least one aromatic or non-aromatic heterocyclic group, at least one hydrocarbylene group (i.e., —CₙH₂ₙ— group, n=1 to about 10), at least on alkyleneoxide group (i.e., —CₙH₂ₙOCₘH₂ₘ, n and m=1 to about 10), or any combination of these linkers. In other embodiments, the end group includes aryl, aromatic or non-aromatic heterocyclic, alkaryl group, alkyl substituted aromatic or non-aromatic heteroaryl groups, or any combination thereof.

### BRIEF DESCRIPTION OF THE DRAWINGS

The disclosure is better understood with reference to the following detailed description together with the appended illustrative drawings in which like elements are numbered the same:

### DEFINITIONS USED IN THE INVENTION

In addition to having their customary and usual meaning, the following definitions apply where the context permits in the specification and claims:

The term “integrin agonist pharmaceutical composition” refers to one or more integrin agonists contained in an integrin agonist carrier composition of this disclosure for administration to a mammal as a medicine.

The term “cell-based therapies” refers to pharmaceutical compositions including one or more cell types, one or more integrin agonists or other chemicals or agents for administration to a mammal as medicine.

The term “therapeutically effective amount” refers to an amount of an agent being administered that will relieve at least to some extent one or more of the symptoms of the disorder being treated. For example, an amount of the compound effective to prevent, alleviate or ameliorate symptoms of disease or prolong the survival of a subject to be treated.

With respect to a disease or disorder, the term “treatment” refers to preventing, deterring the occurrence of the disease or disorder, arresting, regressing, or providing relief from symptoms or side effects of the disease or disorder and/or prolonging the survival of the subject to be treated.

The term “alkyl” as used herein alone or in combination refers to C₁-C₁₂ straight or branched, substituted or unsubstituted saturated chain radicals derived from saturated hydrocarbons by the removal of one hydrogen atom. Representative examples of alkyl groups include methyl, ethyl, n-propyl, iso-propyl, n-butyl, sec-butyl, iso-butyl, and tert-butyl, among others.

The term “alkenyl”, alone or in combination, refers to a substituted or unsubstituted straight-chain or substituted or unsubstituted branched-chain alkenyl radical containing from 2 to 10 carbon atoms. Examples of such radicals include, without limitation, ethenyl, E- and Z-pentenyl, decenyl, and the like.

The term “alkynyl”, alone or in combination, refers to a substituted or unsubstituted straight or substituted or unsubstituted branched chain alkynyl radical containing from 2 to 10 carbon atoms. Examples of such radicals include, without limitation, ethynyl, propynyl, propargyl, butynyl, hexynyl, decynyl, and the like.

The term “lower” modifying “alkyl”, “alkenyl”, “alkynyl” or “alkoxy” refers to a C₁-C₆ unit for a particular functionality. For example, lower alkyl means C₁-C₆ alkyl.

The term “cycloalkyl” as used herein alone or in combination refers to a substituted or unsubstituted aliphatic ring system having 3 to 10 carbon atoms and 1 to 3 rings, including, but not limited to cyclopropyl, cyclopentyl, cyclohexyl, norbornyl, and adamantyl, among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide. This term is meant to encompass cycloalkenyl and cycloalkynyl groups. “Cycloalkyl” includes cis or trans forms. Furthermore, the substituents may either be in endo or exo positions in the bridged bicyclic systems.

The term “cycloalkenyl” as used herein alone or in combination refers to a cyclic carbocycle containing from 4 to 8 carbon atoms and one or more double bonds. Examples of such cycloalkenyl radicals include, without limitation, cyclopentenyl, cyclohexenyl, cyclopentadienyl and the like.

The term “cycloalkylalkyl” as used herein refers to a cycloalkyl group appended to a lower alkyl radical, including, but not limited to cyclohexyl methyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl, among others.

The term “alkoxy”, alone or in combination, refers to an alkyl ether radical, wherein the term “alkyl” is defined above. Examples of suitable alkyl ether radicals include, without limitation, methoxy, ethoxy, n-propoxy, iso-propoxy, n-butoxy, iso-butoxy, sec-butoxy, tert-butoxy and the like.

The term “alkenoxy”, alone or in combination, refers to a radical of formula alkenyl-O—, provided that the radical is not an enol ether, wherein the term “alkenyl” is defined above. Examples of suitable alkenoxy radicals include, without limitation, allyloxy, E- and Z-3-methyl-2-propenoxy and the like.

The term “alkynoxy”, alone or in combination, refers to a radical of formula alkynyl-O—, provided that the radical is not an -ynol ether. Examples of suitable alkynoxy radicals include, without limitation, propargyloxy, 2-butynyloxy and the like.

The term “carboxyl” as used herein refers to —CO₂H.

The term “thioalkoxy”, refers to a thioether radical of formula alkyl-S—, wherein “alkyl” is defined above.

The term “carboxaldehyde” as used herein refers to —C(O)R, wherein R is hydrogen.

The term “carboxamide” as used herein refers to —C(O)NR₂, wherein R is hydrogen, alkyl, or any other suitable substituent.

The term “alkoxyalkoxy” as used herein refers to R_{b} O—R_{c}O—, wherein R_{b} is lower alkyl as defined above and R_{c} is alkylene wherein alkylene is —(CH₂)ₙ— wherein n′ is an integer from 1 to 6. Representative examples of alkoxyalkoxy groups include methoxymethoxy, ethoxymethoxy, and t-butoxymethoxy, among others.

The term “alkylamino” as used herein refers to R_{d} NH—, wherein R_{d} is a lower alkyl group, for example, ethylamino, butylamino, among others.

The term “alkenylamino” alone or in combination, refers to a radical of formula alkenyl-NH— or (alkenyl)₂N—, wherein the term “alkenyl” is defined above, provided that the radical is not an enamine. An example of such alkenylamino radicals is the allylamino radical.

The term “alkynylamino”, alone or in combination, refers to a radical of formula alkynyl-NH— or (alkynyl)₂-, wherein the term “alkynyl” is defined above, provided that the radical is not an amine. An example of such alkynylamino radicals is the propargyl amino radical.

The term “dialkylamino” as used herein refers to RₑR_{f}N—, wherein Rₑ and R_{f} are independently selected from lower alkyl, for example diethylamino, and methyl propylamino, among others.

The term “amino” as used herein refers to H₂N—.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl, among others.

The term “aryl” or “aromatic” as used herein alone or in combination refers to a substituted or unsubstituted carbocyclic aromatic group having about 6 to 12 carbon atoms such as phenyl, naphthyl, indenyl, indanyl, azulenyl, fluorenyl and anthracenyl; or a heterocyclic aromatic group selected from the group consisting of furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl and the like. “Arylalkyl” and “alkylaryl” employ the term “alkyl” as defined above. Rings may be substituted. Aromatic rings may be a fused ring system including other aromatic rings or non-aromatic rings to form multicyclic rings, and are also encompassed by the term “aromatic,” as used herein.

The term “aralkyl”, alone or in combination, refers to an aryl substituted alkyl radical, wherein the terms “alkyl” and “aryl” are defined above. Examples of suitable aralkyl radicals include, without limitation, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “aralkenyl”, alone or in combination, refers to an aryl substituted alkenyl radical, wherein the terms “aryl” and “alkenyl” are defined above.

The term “arylamino”, alone or in combination, refers to a radical of formula aryl-NRg-, wherein “aryl” is defined above. Rg may be selected from the group consisting of H, lower alkyl, aryl and aralkyl, among others. Examples of arylamino radicals include, without limitation, phenylamino(anilido), naphthlamino, 2-, 3-, and 4-pyridylamino and the like.

The term “biaryl”, alone or in combination, refers to a radical of formula aryl-aryl, wherein the term “aryl” is defined above.

The term “thioaryl”, alone or in combination, refers to a radical of formula aryl-S—, wherein the term “aryl” is defined above. An example of a thioaryl radical is the thiophenyl radical.

The term “aroyl”, alone or in combination, refers to a radical of formula aryl-CO—, wherein the term “aryl” is defined above. Examples of suitable aromatic acyl radicals include, without limitation, benzoyl, 4-halobenzoyl, 4-carboxybenzoyl, naphthoyl, pyridylcarbonyl and the like.

The term “heterocyclyl”, alone or in combination, refers to a non-aromatic 3- to 10-membered ring containing at least one endocyclic N, O, or S atom. The heterocycle may be optionally aryl fused. The heterocycle may also optionally be substituted with at least one substituent which is independently selected from the group consisting of hydrogen, halogen, hydroxyl, amino, nitro, trifluoromethyl, trifluoromethoxy, alkyl, aralkyl, alkenyl, alkynyl, aryl, cyano, carboxyl, alkoxycarbonyl, carboxyalkyl, oxo, arylsulfonyl and aralkylaminocarbonyl, among others.

The term “alkylheterocyclyl” as used herein refers to an alkyl group as previously defined appended to the parent molecular moiety through a heterocyclyl group.

The term “heterocyclylalkyl” as used herein refers to a heterocyclyl group as previously defined appended to the parent molecular moiety through an alkyl group.

The term “aminal” as used herein refers to a hemi-acetal of the structure RCH(NH₂)(OH).

The terms “electron-withdrawing” or “electron-donating” refer to the ability of a substituent to withdraw or donate electrons relative to that of hydrogen if hydrogen occupied the same position in the molecule. These terms are well-understood by one skilled in the art and have the meaning described in ADVANCED ORGANIC CHEMISTRY by J. March 1985, pp. 16-18, incorporated herein by reference. Electron withdrawing groups include halo, nitro, carboxyl, lower alkenyl, lower alkynyl, carboxaldehyde, carboxyamido, aryl, quaternary ammonium, trifluoromethyl, and aryl lower alkanoyl, among others. Electron donating groups include such groups as hydroxy, lower alkyl, amino, lower alkylamino, di(lower alkyl)amino, aryloxy, mercapto, lower alkylthio, lower alkylmercapto, and disulfide, among others. One skilled in the art will appreciate that the substituents may have electron donating or electron withdrawing properties under different chemical conditions. Moreover, the present invention contemplates any combination of substituents selected from the above-identified groups.

The most preferred electron donating or electron withdrawing substituents are halo, nitro, alkanoyl, carboxaldehyde, arylalkanoyl, aryloxy, carboxyl, carboxamide, cyano, sulfonyl, sulfoxide, heterocyclyl, guanidine, quaternary ammonium, lower alkenyl, lower alkynyl, sulfonium salts, hydroxy, lower alkoxy, lower alkyl, amino, lower alkylamino, di(lower alkyl)amino, amine lower alkyl mercapto, mercaptoalkyl, alkylthio and alkyldithio.

Use of the above terms is meant to encompass substituted and unsubstituted moieties. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogens, trifluoromethoxy, trifluoromethyl, alkyl, aralkyl, alkenyl, alkynyl, aryl, cyano, carboxy, carboalkoxy, carboxyalkyl, cycloalkyl, cycloalkylalkyl, heterocyclyl, alkylheterocyclyl, heterocyclylalkyl, oxo, arylsulfonyl and aralkylaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)O—. Rings may be substituted.

The term “mammals” includes humans and other animals.

The term “heteroatom” as used herein encompasses nitrogen, sulfur, and oxygen.

The term “alpha” as used herein indicates the position immediately adjacent to the position described.

The term “Log P” means the ratio of the concentrations of a substance in two heterogenous phases in equilibrium with each other. Typically, the organic solvent in n-octanol, but n-hexane; toluene; chlorobenzene; dichloromethane; methyl tert-butyl ether; isopropyl acetate; benzyl alcohol; cyclohexanol; or n-butanol may be used as well.

The term “inactive ingredient” as used herein indicated a harmless drug that may be used as an inactive ingredient, such as a coloring, emulsifier, excipient, flavoring, lubricant, preservative, or solvent, in the preparation of other drugs shall be exempt from section 502(f)(1) of the act (21 CFR 201.117).

The term “gelatin” means a collection of peptides and proteins produced by partial hydrolysis of collagen extracted from the skin, bones, and connective tissues of animals such as domesticated cattle, chicken, pigs, and fish. During hydrolysis, some bonds between and within component proteins are broken. Its chemical composition is, in many aspects, closely like that of its parent collagen. Photographic and pharmaceutical grades of gelatin are sourced from cattle bones and pig skin. Gelatins are classified as a hydrogel.

The term “excipient” or “adjuvant” as used herein means any substance other than the active drug or product which has been appropriately evaluated for safety and is included in a drug delivery system to either aid the processing of the drug delivery system during its manufacture; protect, support, or enhance stability, bioavailability, or patient acceptability; assist in product identification; or enhance any other attribute of the overall safety and effectiveness of the drug delivery system during storage or use (40 CFR 63.1251).

The term “effector cell” as used herein means a cell that has been activated by their cognate tumor-antigen and involved in eliminating a cancer cell. Effector cell types may include: 1) Tumor Infiltrating Lymphocytes (TIL) are lymphocytes isolated from tumors and expanded ex vivo that possess cells surface markers including but not limited CD8 or CD4, 2) T-cell clones reactive to one or plurality of tumor antigens that possess cells surface markers including but not limited CD8 or CD4, 3) T-cells genetically engineered with tumor specific-T-cell receptors or -chimeric antigen receptors that possess cells surface markers including but not limited CD8 or CD4, 4) natural killer cells reactive to a specific or plurality of tumor antigens.

The term “adoptive T-cell” is an effector cell that is derived from a naive T-cell or activated T-cell capable of effector functions.

The term “solid tumor” as used herein means an abnormal mass of tissue that usually does not contain cysts or liquid areas. Solid tumors may be benign (not cancer), or malignant (cancer). Solid tumors are named for the type of cells present in the solid tumor. Examples of solid tumors include, without limitation, sarcomas, carcinomas, and lymphomas.

The term “small molecule agonist” as used herein is not a conventional ligand and is synonymous to a stabilizer of a cognate ligand-receptor interaction.

The term “linker” means a moiety that links two parts of a molecular system together. In the disclosure, the linker are molecular moieties that like to end groups of a small molecule integrin agonist, e.g., end group 1-linker-end group 2. In the integrin agonists of Formula (I), the linkers are defined by -M³-M⁴-M¹-M⁶- and the end group 1 is defined by R¹-M¹-N(R²)-M²- and end group 2 is defined by —R³ In the integrin agonists of Formula (II), the linkers are defined by —R^{a}—Z—R^{b}— and end group 1 is defined by Q¹- and end group 2 is defined by -Q².

The term “at least one”, “one or more” or “one or a plurality” means one or two or more and the three terms may be used interchangeably herein. For example, at least one device, one or more devices, or one or a plurality of device means one device or two devices or many devices, up to some finite number such as the number 20, but higher and lower values may be used.

The term “one or a plurality” means one item or a plurality of items.

The term “about” means that a value of a given quantity is within ±10% of the stated value. In other embodiments, the value is within ±5% of the stated value. In other embodiments, the value is within ±2.5% of the stated value. In other embodiments, the value is within ±1% of the stated value.

The term “substantially” or “essentially” means that a value of a given quantity is within ±5% of the stated value. In other embodiments, the value is within ±2.5% of the stated value. In other embodiments, the value is within ±2% of the stated value. In other embodiments, the value is within ±1% of the stated value. In other embodiments, the value is within ±0.5% of the stated value. In other embodiments, the value is within ±0.1% of the stated value.

The term “substantially free” or “essentially free” means that a value of a given quantity that is ≤5%. In other embodiments, the value is ≤2.5%. In other embodiments, the value is ≤2%. In other embodiments, the value is ≤1%. In other embodiments, the value is ≤0.5%. In other embodiments, the value is ≤0.1%.

All ranges include the endpoint values and any subrange thereof. All single values include all lower values. For example, the term about 25 includes all values greater than zero up to 25.

### ABBREVIATIONS USED IN THE INVENTION

The following abbreviations are used herein: Ac is acetyl, AcOH is acetic acid, 6-Ahx-OH is 6-aminohexanoic acid, Bn is benzyl, Boc is tert-butyloxycarbonyl, nBu is n-butyl, nBuLi is n-butyllithium, 1.6M in hexanes (unless other concentration noted), CBz is benzyloxycarbonyl, CDI is N,N′-carbonyldiimidazole, COMU is (1-cyano-2-ethoxy-2-oxoethylidenaminooxy) dimethylamino-morpholino-carbenium hexafluorophosphate, Dab is 2,4-diaminobutyryl, DBU is 1,8-diazabicyclo[5.4.0]undec-7-ene, DCE is 1,2-dichloroethane, DCHA is dicyclohexylamine, DCM is dichloromethane (methlyene chloride), dioxane is 1,4-dioxane, DIPEA is N,N-diisopropylethylamine, DMED is N,N′-dimethylethylene diamine, DMF is N,N-dimethylformamide, DMSO is dimethylsulfoxide Et is ethyl, EtOH is ethanol, Fmoc is 9H-fluoren-9-ylmethyloxycarbonyl, Glu is glutamic acid, Gly is glycine, HBTU is O-(Benzotriazol-1-yl)-N,N,N′,N′-tetramethyluronium hexafluorophosphate, HMDS is hexamethyldisilazane, iPr is isopropyl, KHMDS is potassium bis(trimethylsilyl)amide, Lys is lysine, LHMDS is lithium bis(trimethylsilyl)amide, Me is methyl, MeOH is methanol, Nle is norleucine, NMM is 4-methylmorpholine, NSMC is N-succinimidyl-N-methylcarbamate, OAc is acetate, Orn is Ornithine, pTsOH is para-toluenesulfonic acid, Ph is phenyl, RT is room temperature, tBu is tert-butyl, TEA is triethylamine, Tfa is trifluoroacetyl, THF is tetrahydrofuran, Tol is toluene, Tyr is tyrosine, and Z is benzyloxycarbonyl.

### DETAILED DESCRIPTION OF THE INVENTION

The inventors have found that integrin agonist carrier compositions may be formulated to improve bioavailability of integrin agonists. The integrin agonist carrier compositions may be used to formulate integrin agonist pharmaceutical compositions for administration to an animal or human. Additionally, the inventors have found that methods for making and using the integrin agonist carrier compositions and the integrin agonist pharmaceutical compositions. The integrin agonist carrier compositions comprising one or more zwitterionic surfactants, one or more neutral lipids, and one or more polar organic solvents.

The inventor has found that integrin agonists, which enhance integrin-mediated intercellular adhesion, may be beneficial in a variety of therapies, especially therapies known or suspected to involve integrin-mediated binding of cells possessing certain integrins and cells possessing the integrins associated ligands. Integrins targeted by integrin agonists include, but are not limited to, α4β1 (very late antigen-4 (VLA-4)), α4β7, α5β1, αLβ2 (Lymphocyte Function-Associated Antigen 1 (LFA-1)), and/or αVβ3 and the corresponding ligands include, but are not limited to, VCAM-1, fibronectin, MAdCAM-1, ICAM-1, ICAM-2 and/or vitronectin.

Small molecule, poorly water-soluble pharmaceutical integrin agonist adjuvant agents and their immunogenic compositions, and methods of using the same to elicit immunological responses in a subject are disclosed herein. The small molecule, poorly water-soluble pharmaceutical integrin agonist adjuvant agents comprise compound(s) that facilitate the interaction of integrins α4β1, α4β7, α5β1, and/or αLβ2 and their cognate ligands. The integrin adjuvant agents described above may be used to potentiate and enhance the immunological effects of active agents, including vaccine antigens derived from various pathogens, toxins, and/or tumors. The practical and preferred route of administration of these integrin agonist adjuvant agents are via the oral route in form of capsules, tablets, solutions, or suspensions.

Ethane-1,2-diylbis(oxy)bis(ethane-2,1-diyl)bis(bis(thiophene-2ylmethyl)carbamate (C1) is a representative small molecule, poorly water-soluble pharmaceutical integrin agonist adjuvant agent with desired pharmacologic properties. However, C1 has limited by aqueous solubility and oral bioavailability. To improve oral absorption, lipid-based carrier compositions have been formulated, and methods to manufacture oral dose forms to reach optimal pharmacokinetic exposures.

Pharmaceutical integrin agonist adjuvant agents of low water solubility, for example those classified as “practically insoluble” or “insoluble” according to United States Pharmacopeia (USP) 24 (2000), p. 10, i.e., having solubility of less than about 1 part per 10,000 parts water (less than about 100 ug/ml) are notoriously difficult to formulate for oral delivery. Among other problems, bioavailability of such drugs, when administered by the oral route, tends to be very low. The oral bioavailability is further confounded by the fact that these molecules have molecular weights >500, without ionizable group, and are metabolized by gastrointestinal mucosal and hepatic CYP45b enzymes.

C1 is a small-molecule, integrin agonist adjuvant agent having low water solubility that was disclosed and used in U.S. Pat. Nos. 9,512,109, 10,071,980, 10,035,784, 10,287,264, 10,342,866, 10,709,780, 10,709,781, 10,716,849, and 11,311,619, and foreign counterparts. Relevant properties of 7HP349 are listed in Table1.

TABLE 1
Small-molecule, Integrin Agonist Adjuvant Agent, 7HP349, Description and Properties

| | |
|---|---|
| Common Name: | 7HP349 |
| Nonproprietary Name: | VLA-4 and LFA-1 integrin allosteric activator |
| Chemical name: | ethane-1,2-diylbis(oxy))bis(ethane-2,1-diyl) |
| | bis(bis(thiophene-2-yl-methyl)carbamate or |
| | 3-oxo-1-(2-thienyl)-2-(2-thienylmethyl)-4,7,10-trioxa-2- |
| | azadodecan-12-yl bis(2-thienylmethyl)carbamate or |
| | 1,16-bis(2-thieny])-2-15-bis(2-thienylmethyl)-3,14-dioxo- |
| | 4,7,10,13-tetraoxa-2,15-diazahexadecane |
| Other non-proprietary name: | VLA-4 (α4ß1) and LFA-1 (αLß2) Integrin Activator |
| Structure: | 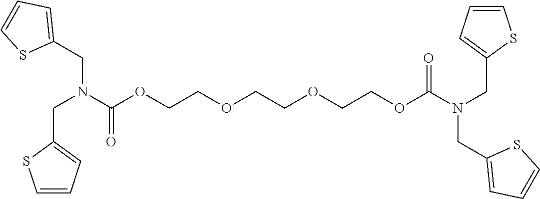 [SMILES: O=C(OCCOCCOCCOC(=O)N(Cc1cccs1)Cc1cccs1)N(Cc1cccs1)Cc1cccs1] |
| Molecular Weight: | 620.82 |
| Physical Description: | Colorless to pale-yellow viscous liquid |
| Solubility: | Insoluble in water |
| | Practically insoluble in non-polar organic solvents |
| | such as hexane, heptane, etc. |
| | Highly soluble in common organic solvents such |
| | as methanol, ethanol, ethyl acetate, acetone, |
| | DMSO, DMF, DCM, etc. |
| | Low solubility (<0.01 μM) in hydrochloric acid- |
| | potassium chloride buffer (pH 2), citrate buffer |
| | (pH 4), sodium phosphate buffer (pH 7.4), SGF, |
| | FaSSIF and FeSSIF |
| | Soluble up to 50 mg/mL in vegetable oils such as |
| | corn, soybean, canola oils typically containing |
| | >C16 fatty acids |
| LogP: | 6.22 |
| Cytochrome P450 Metabolism: | Substrate for CYP3A4 and CYP2C19 |

Another specific illustrative small-molecule drug of low water solubility is the compound 3-oxo-1-(2-thienyl)-2-(2-thienylmethyl)-4,7-dioxa-2-azanonan-9-yl bis(2-thienylmethyl)carbamate or 1,13-bis(2-thienyl)-2-12-bis(2-thienylmethyl)-3,11-dioxo-4,7,10-trioxa-2,12-diazatridecane (7HP577), an integrin agonist adjuvant disclosed in U.S. Pat. Nos. 9,512,109, 10,071,980, 10,035,784, 10,287,264, 10,342,866, 10,709,780, 10,709,781, 10,716,849, and 11,311,619, and foreign counterparts This neutral compound, which has a molecular weight of 576.78 g/mol and Log P value of 6.58.

US20070104780 to Lipari et al disclose a pharmaceutical composition comprises a drug-carrier system having a small-molecule drug of low water solubility, e.g., N-4-(3-amino-1H-indazol-4-yl)phenyl-N′-(2-fluoro-5-methylphenyl)urea (ABT-869) and (+)-1-(5-tert-butyl-1-yl)-3-(1H-indazol-4-yl)-urea (ABT-102), in solution in a substantially non-aqueous carrier that comprises at least one phospholipid and a pharmaceutically acceptable solubilizing agent. The drug carrier system, when mixed with an aqueous phase, typically forms a non-gelling, substantially non-transparent liquid dispersion. The composition is suitable for administration by a suitable route, e.g., orally, to a subject in need thereof.

Caliph et al, J. Pharm Sci, 98, 1073, 2000 disclose the use of carriers including neutral lipids containing fatty acid side chains including greater than 10 carbon atoms (i.e., >C₁₀ fatty acids), optimally neutral lipids containing fatty acid side chains including at least 18 carbon atoms (i.e., ≥C₁₈ fatty acids) improve the lymphatic absorption and oral bioavailability of insoluble compounds such as halofantrine, and Ali Khan et al Int J Nanomedicine, 2013; 8: 2733-2744 disclose such lipid-based formulations may decrease first pass metabolism loss.

There remains a need in the pharmaceutical art for a novel liquid formulation of a small-molecule drug of low water solubility such as 7HP349 and 7HP577 that is suitable for oral administration. More particularly and without limitation, there is a need for Such a formulation having at least one of the following features, advantages, or benefits: acceptably high concentration of integrin agonists such as 7HP349 and 7HP577 (for example at least about 5% w/w); and acceptable bioavailability (for example at least about 5%) when administered orally.

### Lipid-Based Carrier Compositional Ranges

The lipid-based carrier compositions of this disclosure comprise one or more zwitterionic surfactants, one or more neutral lipids, and one or more polar organic co-solvents or solubilizing agents. In certain embodiments, the one or more zwitterionic surfactants comprise one or more phospholipids. Table 2 and Table 3 tabulates ranges for the three principal ingredients of the lipid-based carrier compositions.

TABLE 2
Compositional Ranges for Type 1
Lipid-based Carrier Compositions

| | Ranges | Ranges | Ranges | Ranges |
|---|---|---|---|---|
| Ingredient | (wt. %) | (wt. %) | (wt. %) | (wt. %) |
| surfactants | 100 to 0 | about 10 to | about 30 to | about 40 to |
| | | about 90 | about 70 | about 60 |
| neutral | 0 to 100 | about 90 to | about 70 to | about 60 to |
| lipids | | about 10 | about 30 | about 40 |

All ranges include end points and subranges.

TABLE 3
Compositional Ranges for Lipid-based Carrier Compositions

| | Ranges | Ranges | Ranges | Ranges |
|---|---|---|---|---|
| Ingredient | (wt. %) | (wt. %) | (wt. %) | (wt. %) |
| surfactants | about 5 to | about 5 to | about 5 to | about 5 to |
| | about 94 | about 90 | about 80 | about 70 |
| neutral | about 94 to | about 90 to | about 80 to | about 70 to |
| lipids | about 5 | about 5 | about 5 | about 5 |
| organic | about 1 | about 5 | about 15 | about 25 |
| solvents | | | | |

All ranges include end points and subranges.

The lipid-based carrier compositions may also include up to 10 wt. % of other adjuvants and/or excipients, with the other wt. % adjusted so that the composition adds to 100 wt. %. In other embodiments, the other ingredients include up to 5 wt. %. In other embodiments, the other ingredients include up to 2.5 wt. %. In other embodiments, the other ingredients include up to 1 wt. %.

### Lipid-Based Carrier Pharmaceutical Compositional Ranges

The lipid-based pharmaceutical compositions of this disclosure comprise one or more zwitterionic surfactants, one or more neutral lipids, one or more polar organic co-solvents or solubilizing agents, and one or more small molecule, poorly water-soluble pharmaceutical integrin agonist adjuvant agents. In certain embodiments, the one or more zwitterionic surfactants comprise one or more phospholipids. The following Table 4 tabulates ranges for the four principal ingredients of the lipid-based carrier compositions.

### Table 4

TABLE 4
Compositional Ranges for Lipid-based Carrier Compositions

| | Ranges | Ranges | Ranges | Ranges |
|---|---|---|---|---|
| Ingredient | (wt. %) | (wt. %) | (wt. %) | (wt. %) |
| lipid-based | about 75 to | about 75 to | about 80 to | about 80 to |
| carrier | about 99 | about 97.5 | about 95 | about 90 |
| integrin | about 25 to | about 25 to | about 20 to | about 20 to |
| agonist | about 1 | about 2.5 | about 5 | about 10 |
| adjuvant | | | | |
| agents | | | | |

All ranges include end points and subranges.

The lipid-based pharmaceutical compositions may also include up to 10 wt. % of other adjuvants and/or excipients, with the other wt. % adjusted so that the composition adds to 100 wt. %. In other embodiments, the other ingredients include up to 5 wt. %. In other embodiments, the other ingredients include up to 2.5 wt. %. In other embodiments, the other ingredients include up to 1 wt. %.

### Dosages and Effective Amounts

In certain embodiments, the dosage effective amounts of the poorly water-soluble pharmaceutical agents such as integrin agonists of the disclosure in the lipid-based delivery system are between about 1 milligram (mg) and about 500 mg or any subrange such as between about 1 mg and about 250 mg, between about 1 mg and about 200 mg, between about 1 mg and about 150 mg, between about 1 mg and about 100 mg, between about 1 mg and about 75 mg, between about 1 mg and about 50 mg, between about 1 mg and about 25 mg, between about 5 mg and about 150 mg, between about 5 mg and about 100 nM, between about 5 mg and about 75 mg, between about 5 mg and about 50 mg, between about 5 mg and about 25 mg, or any other subranges thereof.

In certain embodiments, the therapeutically effective amounts of the poorly water-soluble pharmaceutical agents such as integrin agonists of the disclosure at the site of application are between about 1 femto molar (fM) and about 300 micro molar (μM) or any subrange such as between about 1 fM and about 200 μM, between about 1 fM and about 100 PM, between about 1 fM and about 50 μM, between about 1 fM and about 25 μM, between about 1 fM and about 20 μM, between about 1 fM and about 15 μM, between about 1 fM and about 5 μM, between about 1 fM and about 1 μM, between about 1 fM and about 100 nano molar (nM), between about 1 fM and about 75 nM, between about 1 fM and about 50 nM, between about 1 fM and about 25 nM, or any other subranges thereof.

In certain embodiments, the plasma concentrations in nanogram/milliliter (ng/mL) of the poorly water-soluble pharmaceutical agents such as integrin agonists of the disclosure are between about 1 ng/mL and about 25 ng/mL or any subrange such as between about 1 ng/mL and about 20 ng/mL, between about 1 ng/mL and about 15 ng/mL, between about 1 ng/mL and about 5 ng/mL, between about 1 ng/mL and about 1 ng/mL, between about 1 ng/mL and about 100 ng/mL, between about 1 ng/mL and about 75 ng/mL, between about 1 ng/mL and about 50 ng/mL, between about 1 ng/kg and about 25 ng/mL, or any other subranges thereof.

### Poorly Water-Soluble Pharmaceutical Agents

The lipid-based carrier compositions of this disclosure may be used to deliver any poorly water-soluble pharmaceutical agent or mixture thereof to an animal or human. In certain embodiments, the poorly water-soluble pharmaceutical agents comprise poorly water-soluble integrin activating pharmaceutical agents or mixture thereof. Such agents are characterized by possessing a Log P value of greater than 5.0 (>5.0), greater than or equal to 5.25 (≥5.25), greater than or equal to 5.5 (≥5.5), greater than or equal to 5.75 (≥5.75), or greater than or equal to 6.0 (≥6.0).

General Types of Integrin Activating Pharmaceutical Agents

In certain embodiments, the integrin activating pharmaceutical agents are given by compounds of Formula (I):

R¹-M¹-N(R²)-M²-M³-M⁴-M⁵-M⁶-R³ (I)
wherein a first class of the compounds of Formula (I) is defined by:
   R¹ is selected from the group consisting of aryl and aralkyl,
   R² is alkyl, aryl, or aralkyl,
   M¹ is CH₂,
   M² is CO,
   M³ is O, S, or NR⁶,
      R⁶ when present is hydrogen or lower alkyl,
   M⁴ is absent or CH₂,
   M⁵ is (CR¹¹R¹²),
      R¹¹ is hydrogen,
   R¹² is selected from the group consisting of hydrogen, NR²¹CONR²²R²³, NR²¹COR²⁴, NR²¹SO₂R²⁴, NR²¹COOR²⁴, OCOR²⁴, OR²⁴, O(CH₂CH₂O)ₛR²⁴, COOR²⁴, alkyl, and hydroxyalkyl,
      s is an integer of 1 to 6,
      R²¹ and R²² when present are independently selected from the group consisting of hydrogen or lower alkyl,
      R²³ when present is selected from the group consisting of hydroxyalkyl, alkoxyalkyl, alkyl, aryl, aralkyl and alkoxycarbonylalkyl,
         provided that when M³ is NR⁶ and M⁴ is absent, then R²³ is not 1-(1,3-benzodioxol-5-yl)-3-ethoxy-3-oxopropyl,
      R²⁴ when present is selected from the group consisting of alkyl, aryl, aralkyl, heterocyclyl, cycloalkyl, cycloalkylalkyl, and heterocyclylalkyl, and mixtures thereof,
   M⁶ is (CH₂)_{q},
      q is an integer from 0 to 6,
   R³ is selected from the group consisting of hydrogen, CONR¹³R¹⁴, NR¹⁵COOR¹⁶, NR¹⁵COR¹⁶, NR¹⁵CONR¹³R¹⁴, NR¹⁵SO2R¹⁶, OCOR¹⁶, COOR¹⁶, OR¹⁶, SR¹⁶, heterocyclyl, hydroxyl, hydroxyalkyl, guanadino, alkyl and aryl,
      R¹³ and R¹⁵ when present are independently hydrogen or lower alkyl,
      R¹⁴ and R¹⁶ when present are independently selected from the group consisting of hydrogen, alkyl, aryl, aralkyl, cycloalkyl, heterocyclyl, cycloalkylalkyl, and heterocyclylalkyl,
   R¹, R², R³, R¹², R¹⁴, R¹⁶, R²³ and R²⁴ when present may independently be either unsubstituted or substituted with one or more substituents selected from the group consisting of alkyl, aryl, aralkyl, cycloalkyl, cycloalkylalkyl, heterocyclyl, heterocyclylalkyl, heterocyclylaryl, hydroxy, alkoxy, azido, haloalkoxy, hydroxyalkyl, aryloxy, hydroxyaryl, alkoxyaryl, halo, haloalkyl, haloaryl, amino, alkylamino, dialkylamino, arylamino, diarylamino, —NHCO(alkyl), —NHCO(aryl), —NHCO(aralkyl), —NHCO(haloalkyl), —NHSO₂(alkyl), —NHSO₂(aryl), —NHSO₂(aralkyl), alkoxycarbonyl, alkoxycarbonylalkyl, —OCO(alkylamino), —OCO(dialkylamino), and mixtures thereof;
      or
wherein a second class of the compounds of Formula (I) is defined by:
   R¹ is aryl or aralkyl,
   R² is alkyl or aralkyl,
   M¹ is CH₂,
   M² is CO,
   M³ is absent or is O or CH₂,
   M⁴ is absent or is CH₂,
   M⁵ is absent or is O or (CR¹¹R¹²),
      R¹¹ is hydrogen,
      R¹² is selected from the group consisting of hydrogen, NR²¹CONR²²R²³,
      NR²¹COR²⁴, NR²¹SO2R²⁴ and NR²¹COOR²⁴,
         R²¹ and R²² each of which, when present is independently selected from the group of hydrogen and lower alkyl,
         R²³ and R²⁴, each of which, when present is independently selected from the group consisting of hydrogen, alkyl, aryl, cycloalkyl, cycloalkylalkyl, heterocyclyl, heterocyclylalkyl and aralkyl,
   M⁶ is selected from the group consisting of (CH₂)_{q}, (CH₂)_{q}CH═CH—(CH₂)ᵣ, (CH₂)_{q}-arylene-(CH₂)ᵣ and (CH₂CH₂O)_{q}, where
      q and r are independently integers from 0 to 6,
      R³ is CONR¹³R¹⁴,
      R¹³ and R¹⁴, each of which, when present is independently selected from the group consisting of hydrogen, alkyl, aryl, cycloalkyl, cycloalkylalkyl, heterocyclyl, heterocyclylalkyl and aralkyl, and
   R¹, R², R¹³, R¹⁴, R²³ and R²⁴, when present, independently either are unsubstituted or are substituted with one or more substituents selected from the group consisting of alkyl, aryl, aralkyl, cycloalkyl, cycloalkylalkyl, heterocyclyl, heterocyclylalkyl, heterocyclylaryl, hydroxy, alkoxy, azido, haloalkoxy, hydroxyalkyl, aryloxy, hydroxyaryl, alkoxyaryl, halo, haloalkyl, haloaryl, amino, alkylamino, dialkylamino, arylamino, diarylamino, —NHCO(alkyl), —NHCO(aryl), —NHCO(aralkyl), —NHCO(haloalkyl), —NHSO₂(alkyl), —NHSO₂(aryl), —NHSO₂(aralkyl), alkoxycarbonyl, alkoxycarbonylalkyl, —OCO(alkylamino), —OCO(dialkylamino), and mixtures thereof;
or
wherein a third class of the compounds of Formula (I) is defined by:
   R¹ is aryl or aralkyl,
   R² is alkyl or aralkyl,
   M¹ is CH₂,
   M² is SO₂ or CO,
   M³ is absent or is CH₂,
   M⁴ is absent or is CH₂,
   M⁵ is absent or is (CR¹¹R¹²),
      R¹¹, when present, is hydrogen,
      R¹², when present, is selected from the group consisting of hydrogen,
      alkyl, NR²¹CONR²²R²³, NR²¹COR²⁴, NR²¹SO2R²⁴ and NR²¹COOR²⁴,
         R²¹ and R²², each of which when present, is independently selected from the group of hydrogen, lower alkyl, and aralkyl,
         R²³ and R²⁴, each of which, when present is independently selected from the group consisting of hydrogen, alkyl, aryl, cycloalkyl, cycloalkylalkyl, heterocyclyl, heterocyclylalkyl and aralkyl,
   M⁶ is (CH₂)_{q}, or NR³⁴(CH₂)_{q},
      q is an integer from 0 to 6,
      R³⁴, when present, is selected form the group consisting of alkyl, aralkyl, COR³⁵, and SO₂R³⁵,
         R³⁵ when present, is selected form the group consisting of alkyl, aryl, and aralkyl, and
   R³ is selected from the group consisting of CONR¹³R¹⁴, SO₂NR¹³R¹⁴, NR¹⁵COOR¹⁶, NR¹⁵COR¹⁶, NR¹⁵CONR¹³R¹⁴, and NR¹⁵SO₂R¹⁶,
      R¹³ and R^{M}, each of which, when present, is independently selected from the group consisting of hydrogen, alkyl, aryl, cycloalkyl, cycloalkylalkyl, heterocyclyl, heterocyclylalkyl and aralkyl,
      R¹⁵ and R¹⁶, each of which when present, is independently selected from the group of hydrogen, lower alkyl, and aralkyl,
   R¹, R², R¹³, R¹⁴, R¹⁵, R¹⁶, R²³, R²⁴, R³⁴ and R³⁵, when present, either are unsubstituted or are substituted with one or more substituents selected from the group consisting of alkyl, aryl, aralkyl, cycloalkyl, cycloalkylalkyl, heterocyclyl, heterocyclylalkyl, heterocyclylaryl, hydroxy, alkoxy, azido, haloalkoxy, hydroxyalkyl, aryloxy, hydroxyaryl, alkoxyaryl, halo, haloalkyl, haloaryl, amino, alkylamino, dialkylamino, arylamino, diarylamino, —NHCO(alkyl), —NHCO(aryl), —NHCO(aralkyl), —NHCO(haloalkyl), —NHSO₂(alkyl), —NHSO₂(aryl), —NHSO₂(aralkyl), alkoxycarbonyl, alkoxycarbonylalkyl, —OCO(alkylamino), and —OCO(dialkylamino),
   with the proviso that when M² is CO, then M⁶ is NR³⁴(CH₂)_{q},
      wherein q is not 0;
or
wherein a four class of the compounds of Formula (I) is defined by:
   R¹ is alkyl, aryl or aralkyl,
   R² is selected from the group consisting of aralkyl and alkyl,
      provided that when R¹ is alkyl, R² is aralkyl,
   M¹ is CO or SO₂,
      provided that when M¹ is SO₂ and R¹ is phenyl, 4-methylphenyl or 2,4,6-trimethylphenyl, R² is not alkyl, 2-phenethyl, benzyl, or 2-methoxy-2-oxoethyl, and when M¹ is CO and R¹ is 2-furyl, 4-pyridyl, or 3,5-dinitrophenyl, R² is not alkyl, benzyl or 2-(1H-indol-2-yl)ethyl,
   M² is absent or CH₂,
   M³ and M⁴ are absent,
   M⁵ is (CR¹¹R¹²),
      R¹¹ is hydrogen,
      R¹² is selected from the group consisting of hydrogen, NR²¹CONR²²R²³, NR²¹COR²⁴, NR²¹SO₂R²⁴, NR²¹COOR²⁴, CONR²²R²³, COOR²⁴, O(CH₂CH₂O)ₛR²⁴, hydroxyalkyl, and alkoxyalkyl,
         R²¹, and R²², when present, are independently selected from the group consisting of hydrogen and C₁-C₆ alkyl, and
         R²³ and R²⁴, each of which, when present, is independently selected from the group consisting of hydrogen, alkyl, aryl, cycloalkyl, cycloalkylalkyl, heterocyclyl, heterocyclylalkyl and aralkyl, and
         s is an integer of 1 to 6,
   M⁶ is (CH₂)_{q},
      q is an integer of 0 to 6,
   R³ is selected from the group consisting of NR¹⁵COOR¹⁶, NR¹⁵COR¹⁶, NR¹⁵CONR¹³R¹⁴, and NR¹⁵SO₂R¹⁶, and
      R¹³ when present, is independently selected from the group consisting of hydrogen and C₁-C₆ alkyl, and
      R¹⁴, R¹⁵, and R¹⁶ each of which, when present, are independently selected from the group consisting of hydrogen, alkyl, aryl, cycloalkyl, cycloalkylalkyl, heterocyclyl, heterocyclylalkyl and aralkyl, and
   R¹, R², R³, R¹², R¹⁴, R¹⁵, R¹⁶, R²³, and R²⁴, when present, independently either are unsubstituted or are substituted with one or more substituents selected from the group consisting of alkyl, aryl, aralkyl, cycloalkyl, cycloalkylalkyl, heterocyclyl, heterocyclylalkyl, heterocyclylaryl, hydroxy, alkoxy, azido, haloalkoxy, hydroxyalkyl, aryloxy, hydroxyaryl, alkoxyaryl, halo, haloalkyl, haloaryl, amino, alkylamino, dialkylamino, arylamino, diarylamino, —NHCO(alkyl), —NHCO(aryl), —NHCO(aralkyl), —NHCO(haloalkyl), —NHSO₂(alkyl), —NHSO₂(aryl), —NHSO₂(aralkyl), alkoxycarbonyl, alkoxycarbonylalkyl, —OCO(alkylamino) and —OCO(dialkylamino),
or
pharmaceutically acceptable salts thereof,
or
mixtures thereof.

In some embodiments, a chemical compound is provided having the general formula (I), where R¹ is aryl or aralkyl, R² is alkyl, aryl or aralkyl, M¹ is CH₂, M² is CO, M³ is absent, M⁴ is absent or is CH₂, M⁵ is (CR¹¹R¹²), M⁶ is (CH₂)_{q}, wherein q is an integer of 0 to 6, R¹¹ is hydrogen, and R¹² is selected from the group consisting of hydrogen, NR²¹CONR²²R²³, NR²¹COR²⁴, NR²¹SO₂R²⁴, NR²¹COOR²⁴, OCOR²⁴, OR²⁴, SCOR²⁴, SR²⁴, N₃, CN, and O(CH₂CH₂O)ₛR²⁴, wherein s is an integer of 1 to 6, R²¹ and R²² when present are independently selected from the group consisting of hydrogen, lower alkyl, or aralkyl, R²³ when present is selected from the group consisting of hydroxyalkyl, alkoxyalkyl, alkyl, aryl, aralkyl, and alkoxycarbonylalkyl, R²⁴ when present is selected from the group consisting of alkyl, aryl, aralkyl, heterocyclyl, cycloalkyl, cycloalkylalkyl and heterocyclylalkyl, provided that when M³ and M⁴ are absent, R¹² is not of the formula:

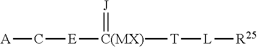

where A is selected from the group consisting of —O—, —S—, and —NR²⁶—, E is selected from the group consisting of —CH₂—, —O—, —S—, and —NR²⁷—, J is selected from the group consisting of —O—, —S—, and —NR²⁸—, T is selected from the group consisting of CO and (CH₂)_{b} wherein b is an integer of zero to three, L is selected from the group consisting of —(CH₂)—, —O—, —S—, and —NR²⁹— wherein n is an integer of zero to three, M is selected from the group consisting of CR³⁰R³¹ and (CH₂)—, wherein u is an integer of zero or one, X is selected from the group consisting of CO₂B, PO₃H₂, SO₃H, OPO₃H₂, CONHCOR³², CONHSO₂R³³, oxazolyl, tetrazolyl and hydrogen, B, R²⁵, R²⁶, R²⁷, R²⁸, R²⁹, R³⁰, R³¹, R³² and R³³ are independently selected from the group consisting of hydrogen, halogen alkyl, alkenyl, alkynyl, alkoxy, alkenoxy, alkynoxy, thioalkoxy, aliphatic acyl, —CF₃, nitro, amino, cyano, N(C₁-C₃ alkyl)CO(C₁-C₃ alkyl), C₁-C₃ alkylamino, alkenylamino, alkynylamino, di(C₁-C₃ alkyl)amino, CO₂(C₁-C₃ alkylamino), CONH(C₁-C₃ alkylamino), CH═NOH, PO₃H₂, OPO₃H₂, CON(C₁-C₃ alkyl)₂, haloalkyl, alkoxycarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkenyl, aralkyl, alkylheterocyclyl, heterocyclycalkyl, sulfonyl, sulfonamide, carbamate, aryloxyalkyl, carboxyl and CONH(benzyl), wherein B, X, R²⁵, R²⁶, R²⁷, R²⁸, R²⁹, R³⁰, R³¹ and R³² are unsubstituted or substituted with at least one electron donating or electron withdrawing group, R³ is selected from the group of hydrogen, NR¹⁵COOR¹⁶, NR¹⁵COR¹⁶, NR¹⁵CONR¹³R¹⁴, NR¹⁵SO₂R¹⁶, OCOR¹⁶, COOR¹⁶, alkyl, SR¹⁶, heterocyclyl, hydroxyl, hydroxyalkyl, guanadino and aryl, wherein R¹³ and R¹⁵ when present are independently hydrogen, lower alkyl, or aralkyl, R¹⁴ and R¹⁶ when present are independently selected from the group consisting of hydrogen, alkyl, aralkyl, cycloalkyl, cycloalkylalkyl, heterocyclyl and heterocyclylalkyl provided that when R³ is hydrogen, alkyl or aryl, R¹² is not hydrogen, and provided that when R¹ is phenyl, R³ is benzyloxycarbonylamino, and R¹² is hydrogen, R² is not 2-methoxybenzyl, and R¹, R², R³, R¹², R¹³, R¹⁴, R¹⁵, R¹⁶, R²¹, R²², R²³ and R²⁴ when present may independently be either unsubstituted or substituted with one or more substituents selected from the group consisting of alkyl, aryl, aralkyl, cycloalkyl, cycloalkylalkyl, heterocyclyl, heterocyclylalkyl, heterocyclylaryl, hydroxy, alkoxy, azido, hydroxyalkyl, aryloxy, hydroxyaryl, alkoxyaryl, halo, haloalkyl, haloaryl, haloalkoxy, amino, alkylamino, dialkylamino, arylamino, diarylamino, —NHCO(alkyl), —NHCO(aryl), —NHCO(aralkyl), —NHCO(haloalkyl), —NHSO₂(alkyl), —NHSO₂(aryl), —NHSO₂(aralkyl), alkoxycarbonyl, alkoxycarbonylalkyl, —OCO(alkylamino), —OCO(dialkylamino).

In other embodiments, the integrin activating pharmaceutical agents are defined by compounds of Formula (II):

Q¹-R^{a}—Z—R^{b}-Q² (II)

wherein:
the Q¹ and Q² groups may independently be an R¹R²N— group, an R¹R²NC(═O)— group, an R¹R²NC(═O)N(R³)— group, an R¹R²NC(═O)O— group, or an R¹R²NSO₂— group,
   the R¹ and R² groups may independently be a hydrocarbyl group, a heterohydrocarbyl group, an aryl-containing hydrocarbyl group, a heteroaryl-containing hydrocarbyl group, an aryl-containing heterohydrocarbyl group, a heteroaryl-containing heterohydrocarbyl group, a fused heterocyclic ring group, or any combination thereof, and
   the R³ group may be a hydrocarbyl group or a heterohydrocarbyl group;
the R^{a} and R^{b} groups may independently be a hydrocarbenyl linking group, wherein one or more carbon atoms may be replaced by oxygen atoms, e.g., an alkyleneoxide linking group such as a methyleneoxide containing linking group or an ethyleneoxide containing linking group; and
the Z group may be a hydrocarbenyl linking group or a heterohydrocarbenyl linking group and includes one or more protonatable moieties.

In certain embodiments, the one or more protonatable moieties become protonated at biological pHs and/or are protonated and include pharmaceutically acceptable counterions.

In certain embodiments, the R¹ and R² groups are selected from the groups consisting of an 2-thienylalkyl group, an 3-alkoxybenzyl group, an 4-alkoxybenzyl group, an pyridin-2-ylalkyl, pyridin-4-ylalkyl group, an pyridin-4-ylalkyl group, an 4-dialkylaminobenzyl group, an 3-dialkylaminobenzyl group, and mixture or combinations thereof, where the alkyl or alkoxy groups independently include 1 to 6 carbon atoms.

In other embodiments, the R¹ and R² groups are selected from the groups consisting of an 2-thienylmethyl group, an 2-(2-thienyl)ethyl group, an 3-methoxybenzyl group, an 4-methoxybenzyl group, an pyridin-2-ylmethyl group, an pyridin-4-ylmethyl group, an pyridin-4-ylmethyl group, an 4-dimethylaminobenzyl group, an 3-dimethylaminobenzyl, carbazole, 3,6-dimethoxycarbazole, and mixture or combinations thereof.

In certain embodiments, the R^{a} and R^{b} groups may independently be an —O(R^{c}O)ₙ—, —R^{d}O(R^{c}O)ₙ— group, an —O(R^{c}O)ₙR^{e}-group, an —R^{d}O(R^{c}O)ₙR^{e}— group, or an R^{aa} group, wherein R^{c}, R^{d}, R^{e}, and R^{aa} may independently be hydrocarbyl linking groups, and each n is independently an integer having a value of 1 to 6. In other embodiments, the R^{a} and R^{b} groups may independently be —O((CH₂)ₘO)ₙ—, where m is an integer having a value of 1 to 3 and n is an integer having a value or 1 to 6.

In other embodiments, the R^{a} and R^{b} groups may independently be an —O((CH₂)ₘ₁)((CH₂)ₘ₂O)ₙ(CH₂)ₘ₃— group, where m1, m2 and m3 are integers having values of 1 to 3 and n is an integer having a value of 1 to 8. In other embodiments, the R^{a} and R^{b} groups may independently be an —((CH₂)ₘ₁)((CH₂)ₘ₂O)ₙ(CH₂)ₘ₃— group or —(CH₂)ₘ₁—, where m1, m2 and m3 are integers having values of 1 to 3 and n is an integer having a value of 1 to 6. Illustrative examples include, without limitation, an —O(CH₂O)ₙ— group, an —O(CH₂CH₂O)ₙ— group, an —O(CH₂CH₂CH₂O)ₙ-group, an —CH₂O(CH₂O)ₙ-group, an —O(CH₂O)ₙCH₂-group, an —CH₂O(CH₂O)ₙCH₂-group, an —CH₂O(CH₂CH₂O)ₙ-group, an —O(CH₂CH₂O)ₙCH₂-group, an —CH₂O(CH₂CH₂O)ₙCH₂-group, an —CH₂CH₂O(CH₂CH₂O)ₙ-group, an —O(CH₂CH₂O)ₙCH₂CH₂-group, an —CH₂CH₂O(CH₂CH₂O)ₙCH₂CH₂-group, or higher analogs, or —(CH₂)ₙ— group, wherein n is an integer having a value between 1 and 6. It should be recognized that the choice of R^{a} and R^{b} will depend on the choice of Q¹ and Q² so that the agonists do not include certain moieties such as an —C—N—O— moiety, an —O—O— moiety, or other linkages that are unstable or breakdown into undesirable by products.

In certain embodiments, the Z group may be an —R^{f}N(R⁴)R^{g}— group, an —R^{f}N⁺(R⁴R⁵A⁻)R^{g}— group, an —R^{f}C(R⁶)(N(R⁴R⁵))R^{g}— group, an —R^{f}C(R⁶)(N(R⁴R⁵R⁷A⁻))R^{g}— group, an —R^{f}C(R⁶)(R^{h}N(R⁴R⁵))R^{g}— group, or an —R^{f}C(R⁶)(RhN(R⁴R⁵R⁷A⁻))R^{g}— group, wherein (a) the R⁴, R⁵, R⁶, and R⁷ groups are independently hydrocarbyl groups or heterohydrocarbyl groups, (b) the R^{f} and R^{g} groups are independently C₁-C₃ alkenyl linking group, and (c) the A⁻ groups are independently counterions.

In other embodiments, the Z group comprises an -G¹-J-G²- group, wherein (a) the J group comprises an arylene group or a heteroarylene group; (b) the G¹ and G² groups are independently an —R^{f}—(R⁸)N— group, an —R^{f}—(R⁸)N—R⁹— group, an —R^{f}—O— group, an —R^{f}—O—R^{g}— group, an —C(═O)— group, an —C(═O)—R^{g}— group, an —C(═O)N(R^{g})— group, an —C(═O)N(R⁸)—R^{g}— group, an —C(═O)O— group, an —C(═O)O—R^{g}— group, an —R^{f}—(R⁸)NC(═O)— group, an —R^{f}—(R⁸)NC(═O)—R^{g}— group, an —R^{f}—(R⁸)NC(═O)N(R⁸)— group, an —R^{f}—(R⁸)NC(═O)N(R⁸)—R^{g}— group, an —R^{f}—(R⁸)NC(═O)O— group, an —R^{f}—(R⁸)NC(═O)O—R^{g}— group, an —R^{f}OC(═O)— group, an —R^{f}—OC(═O)—R^{g}— group, an —R^{f}—OC(═O)N(R⁸)— group, an —R^{f}—OC(═O)N(R⁸)—R^{g}— group, an —R^{f}—OC(═O)O— group, or an —R^{f}—OC(═O)O—R^{g}— group; (c) the R^{f} and R^{g} groups are independently C₁-C₃ alkenyl linking group, (d) the R⁸ groups are independently a hydrogen atom or a C₁-C₈ hydrocarbyl group, and (e) the A⁻ groups independently comprise counterions.

In certain embodiments, the Z group may include a hydrocarbyl or a heterohydrocarbyl linking group including any of the hydrocarbyl or a heterohydrocarbyl linking group disclosed herein.

In other embodiments, the Z group may include a hydrocarbyl or a heterohydrocarbyl group, wherein the hydrocarbyl or a heterohydrocarbyl group includes one or more moieties that protonate at biological pHs and/or bear a charge in association with an acceptable counterion. Illustrative examples of Z groups comprising a hydrocarbyl group or a heterohydrocarbyl group including at least one moiety that protonates at biological pHs and/or bears a charge in association with an acceptable counterion include, without limitation, groups derived from pyrrole, pyrrole-2,3-dicarboxylic acid, pyridine, pyridine-2,3-dicarboxylic acid, pyridine-2,4-dicarboxylic acid, pyridine-2,5-dicarboxylic acid, pyridine-3,4-dicarboxylic acid, pyridine-2,4-dicarboxylic acid, pyridine-2,5-dicarboxylic acid, pyridine-2,6-dicarboxylic acid, pyridine-3,4-dicarboxylic acid, pyridine-3,5-dicarboxylic acid, or other heterohydrocarbyl groups. In other embodiments, the Z group may be derived from a hydrocarbyl group include, without limitation, 1-amino-benzene-2,4-dicarboxylic acid, 1-amino-benzene-2,5-dicarboxylic acid, 1-amino-benzene-2,6-dicarboxylic acid, 1-amino-benzene-3,4-dicarboxylic acid, 1-amino-benzene-3,5-dicarboxylic acid, 1,2,3-trihydroxybenzene, 1,2,4-trihydroxybenzene, 1,2,5-trihydroxybenzene, 1,3,4-trihydroxybenzene, 1,3,5-trihydroxybenzene, 1-amino-2,3-dihydroxybenzene, 1-amino-2,4-dihydroxybenzene, 1-amino-2,5-dihydroxybenzene, 1-amino-3,4-dihydroxybenzene, 1-amino-3,5-dihydroxybenzene, orthoformic acid, glycerol, 2-amino-1,3-dihydroxypropane, diethanolamine, N-methyldiethanolamine, dipropanolamine, N-methyldipropanolamine, diisopropanolamine, N-methyldiisopropanolamine, higher dialkanolamines, higher N-methyl dialkanolamines, or other hydrocarbyl groups.

In certain embodiments, when present and not a hydrogen atom, the R¹, R², R³, R⁴, R⁵, R⁶, R⁷, and R⁸ groups may independently be either unsubstituted or substituted with one or more substituents selected from the group consisting of an alkyl group, an aryl group, an aralkyl group, a cycloalkyl group, a cycloalkylalkyl group, a heterocyclyl group, a heterocyclylalkyl group, a heterocyclylaryl group, a hydroxy group, an alkoxy group, an azido group, a haloalkoxy group, a hydroxyalkyl group, an aryloxy group, a hydroxyaryl group, an alkoxyaryl group, a halogen atom, a haloalkyl group, a haloaryl group, an amino group, an alkylamino group, a dialkylamino group, an arylamino group, a diarylamino group, an —NHC(═O)(alkyl) group, an —NHC(═O)(aryl) group, an —NHC(═O)(aralkyl) group, an —NHC(═O)(haloalkyl) group, an —NHSO₂(alkyl) group, an —NHSO₂(aryl) group, an —NHSO₂(aralkyl) group, an alkoxycarbonyl group, an alkoxycarbonylalkyl group, an —OC(═O)(alkylamino) group, and an —OC(═O)(dialkylamino) group.

### Specific Integrin Activating Pharmaceutical Agents of Formulas (I)

In some embodiments, the integrin agonists of Formula (I) comprise:
N,N,N′,N′-tetrakis(2-thienylmethyl)pentanediamide;
N-(3-methoxybenzyl)-N,N′,N′-tris(2-thienylmethyl)pentanediamide;
N,N,N′-tris(2-thienylmethyl)pentanediamide;
N′-[2-(2-thienyl)ethyl]-N,N-bis(2-thienylmethyl)pentanediamide;
N-[2-(2-thienyl)ethyl]-N,N′,N′-tris (2-thienylmethyl)pentanediamide;
N,N-bis(pyridin-4-ylmethyl)-N′,N′-bis(2-thienylmethyl) pentanediamide;
N,N-bis(pyridin-3-ylmethyl)-N′,N′-bis(2-thienylmethyl)pentanediamide;
N,N-bis(3-methoxybenzyl)-N′,N′-bis(2-thienylmethyl)pentanediamide;
N,N,N′,N′-tetrakis(4-methoxybenzyl)pentanediamide;
N,N,N′,N′-tetrakis(2-thienylmethyl) hexanediamide;
N,N,N′,N′-tetrakis(4-methoxybenzyl)hexanediamide;
N,N,N′,N′-tetrakis (3-methoxybenzyl)hexanediamide;
N,N,N′,N′-tetrakis(2-thienylmethyl)heptanediamide;
2,2′-(1,3-phenylene)bis[N,N-bis(2-thienylmethyl)acetamide];
N,N,N′,N′-tetrakis (4-methoxybenzyl)heptanediamide;
N,N,N′,N′-tetrakis(2-thienylmethyl)octanediamide;
(3E)-N,N,N′,N′-tetrakis(2-thienylmethyl)hex-3-enediamide;
2,2′-oxybis[N,N-bis(2-thienylmethyl) acetamide];
3-oxo-1-(2-thienyl)-2-(2-thienylmethyl)-4,7,10-trioxa-2-azadodecan-12-yl bis(2-thienylmethyl)carbamate (7HP349);
3-oxo-1-(2-thienyl)-2-(2-thienylmethyl)-4,7-dioxa-2-azanonan-9-yl bis(2-thienylmethyl)carbamate (7HP577);
N,N,N′,N′-tetrakis(4-methoxybenzyl)succinamideethane-1,2-diyl bis[bis(2-thienylmethyl)carbamate]; N,N,N′,N′-tetrakis(4-methoxybenzyl)octanediamide;
N,N,N′,N′-tetrakis(2-thienylmethyl)pyridine-3,5-dicarboxamide;
N,N,N′,N′-tetrakis (2-thienylmethyl)pyridine-2,6-dicarboxamide;
N,N,N′,N′-tetrakis(2-thienylmethyl) pyridine-2,4-dicarboxamide;
2,2′-(1,4-phenylene) bis[N,N-bis(2-thienylmethyl)acetamide];
8-{2-[bis(2-thienylmethyl)amino]-2-oxoethoxy}-N,N-bis(2-thienylmethyl)quinoline-2-carboxamide;
N,N′-bis(4-methoxybenzyl)-N,N′-bis(2-thienylmethyl)hexanediamide; and
tert-butyl{(2S)-1,6-bis[bis(2-thienylmethyl)amino]-1,6-dioxohexan-2-yl}carbamate.

### Specific Integrin Activating Pharmaceutical Agents of Formulas (II)

### R^{a}ZR^{b}═(OCH₂)ₙN(CH₃)(CH₂O) Class

In certain embodiments, the integrin agonists of Formula (II) comprise one or more agonists, wherein the R^{a}ZR^{b} group comprises —(OCH₂)ₙN(CH₃)(CH₂O)ₙ—, wherein n is an integer between 1 and 6. In other embodiments, the agonists of Formula (II) comprise one or more of the following integrin activating compounds:
(a) 3-oxo-1-(2-thienyl)-2-(2-thienylmethyl)-4-oxa-2,6-diaza-6-methyl-heptan-7-yl-bis(2-thienylmethyl)carbamate or 3,9-dioxo-1,11-bis(2-thienyl)-2,10-bis(2-thienylmethyl)-4,8-dioxa-2,6,10-triaza-6-methyl-undecane,
(b) 3-oxo-1-(2-thienyl)-2-(2-thienylmethyl)-4,6,10-trioxa-2,8-diaza-8-methyl-undecan-11-yl-bis(2-thienylmethyl)carbamate or 3,13-dioxo-1,15-bis(2-thienyl)-2,14-bis(2-thienylmethyl)-4,6,10,12-tetraoxa-2,8,14-triaza-8-methyl-pentadecane,
(c) 3-oxo-1-(2-thienyl)-2-(2-thienylmethyl)-4,6,8,12,14-pentaoxa-2,10-diaza-10-methyl-pentadecan-15-yl-bis(2-thienylmethyl)carbamate or 3,17-dioxo-1,19-bis(2-thienyl)-2,18-bis(2-thienylmethyl)-4,6,8,12,14,16-hexaoxa-2,10,18-triaza-10-methyl-nonadecane,
(d) 3-oxo-1-(3-methoxyphenyl)-2-(3-methoxybenzyl)-4-oxa-2,6-diaza-6-methyl-heptan-7-yl-bis(3-methoxybenzyl)carbamate or 3,9-dioxo-1,11-bis(3-methoxyphenyl)-2,10-bis(3-methoxybenzyl)-4,8-dioxa-2,6,10-triaza-6-methyl-undecane,
(e) 3-oxo-1-(3-methoxyphenyl)-2-(3-methoxybenzyl)-4,6,10-trioxa-2,8-diaza-8-methyl-undecan-11-yl-bis(3-methoxybenzyl)carbamate or 3,13-dioxo-1,15-bis(3-methoxyphenyl)-2,14-bis(3-methoxybenzyl)-4,6,10,12-tetraoxa-2,8,14-triaza-8-methyl-pentadecane,
(f) 3-oxo-1-(3-methoxyphenyl)-2-(3-methoxybenzyl)-4,6,8,12,14-pentaoxa-2,10-diaza-10-methyl-pentadecan-15-yl-bis(3-methoxybenzyl)carbamate or 3,17-dioxo-1,19-bis(2-thienyl)-2,18-bis(3-methoxybenzyl)-4,6,8,12,14,16-hexaoxa-2,10,18-triaza-10-methyl-nonadecane,
(g) 3-oxo-1-(3-methoxyphenyl)-2-(4-dimethylaminobenzyl)-4-oxa-2,6-diaza-6-methyl-heptan-7-yl-(3-methoxybenzyl)(4-dimethylaminobenzyl)carbamate or 3,9-dioxo-1,11-bis(3-methoxyphenyl)-2,10-bis(3-methoxybenzyl)-4,8-dioxa-2,6,10-triaza-6-methyl-undecane,
(h) 3-oxo-1-(3-methoxyphenyl)-2-(4-dimethylaminobenzyl)-4,6,10-trioxa-2,8-diaza-8-methyl-undecan-11-yl-(3-methoxybenzyl)(4-dimethylaminobenzyl)carbamate or 3,13-dioxo-1,15-bis(3-methoxyphenyl)-2,14-bis(3-methoxybenzyl)-4,6,10,12-tetraoxa-2,8,14-triaza-8-methyl-pentadecane,
(i) 3-oxo-1-(3-methoxyphenyl)-2-(4-dimethylaminobenzyl)-4,6,8,12,14-pentaoxa-2,10-diaza-10-methyl-pentadecan-15-yl-(3-methoxybenzyl)(4-dimethylaminobenzyl)carbamate or 3,17-dioxo-1,19-bis(2-thienyl)-2,18-bis(3-methoxybenzyl)-4,6,8,12,14,16-hexaoxa-2,10,18-triaza-10-methyl-nonadecane,
(j) 3-oxo-1-(4-methoxyphenyl)-2-(4-methoxybenzyl)-4-oxa-2,6-diaza-6-methyl-heptan-7-yl-bis(4-methoxybenzyl)carbamate or 3,9-dioxo-1,11-bis(4-methoxyphenyl)-2,10-bis(4-methoxybenzyl)-4,8-dioxa-2,6,10-triaza-6-methyl-undecane,
(k) 3-oxo-1-(4-methoxyphenyl)-2-(4-methoxybenzyl)-4,6,10-trioxa-2,8-diaza-8-methyl-undecan-11-yl-bis(4-methoxybenzyl)carbamate or 3,13-dioxo-1,15-bis(4-methoxyphenyl)-2,14-bis(4-methoxybenzyl)-4,6,10,12-tetraoxa-2,8,14-triaza-8-methyl-pentadecane,
(l) 3-oxo-1-(4-methoxyphenyl)-2-(4-methoxybenzyl)-4,6,8,12,14-pentaoxa-2,10-diaza-10-methyl-pentadecan-15-yl-bis(4-methoxybenzyl)carbamate or 3,17-dioxo-1,19-bis(4-methoxyphenyl)-2,18-bis(4-methoxybenzyl)-4,6,8,12,14,16-hexaoxa-2,10,18-triaza-10-methyl-nonadecane,
(m) 3-oxo-1-(4-methoxyphenyl)-2-(4-dimethylaminobenzyl)-4-oxa-2,6-diaza-6-methyl-heptan-7-yl-(4-methoxybenzyl)(4-dimethylaminobenzyl)carbamate or 3,9-dioxo-1,11-bis(4-methoxyphenyl)-2,10-bis(4-methoxybenzyl)-4,8-dioxa-2,6,10-triaza-6-methyl-undecane,
(n) 3-oxo-1-(4-methoxyphenyl)-2-(4-dimethylaminobenzyl)-4,6,10-trioxa-2,8-diaza-8-methyl-undecan-11-yl-(4-methoxybenzyl)(4-dimethylaminobenzyl)carbamate or 3,13-dioxo-1,15-bis(4-methoxyphenyl)-2,14-bis(4-methoxybenzyl)-4,6,10,12-tetraoxa-2,8,14-triaza-8-methyl-pentadecane,
(o) 3-oxo-1-(4-methoxyphenyl)-2-(4-dimethylaminobenzyl)-4,6,8,12,14-pentaoxa-2,10-diaza-10-methyl-pentadecan-15-yl-(4-methoxybenzyl)(4-dimethylaminobenzyl)carbamate or 3,17-dioxo-1,19-bis(2-thienyl)-2,18-bis(4-methoxybenzyl)-4,6,8,12,14,16-hexaoxa-2,10,18-triaza-10-methyl-nonadecane,
(p) N-methylbis[(9-carbazolylcarbonyloxy)methyl]amine,
(q) N-methylbis{[(9-carbazolylcarbonyloxy)methoxy]methyl}amine,
(r) N-methylbis({[(9-carbazolylcarbonyloxy)methoxy]methoxy}methyl)amine,
(s) N-methylbis[(3,6-dimethoxy-9-carbazolylcarbonyloxy)methyl]amine,
(t) N-methylbis{[(3,6-dimethoxy-9-carbazolylcarbonyloxy)methoxy]methyl}amine,
(u) N-methylbis({[(3,6-dimethoxy-9-carbazolylcarbonyloxy)methoxy]methoxy}methyl)amine,
(v) higher analogs, or
(w) mixtures and combinations thereof.

In other embodiments, the integrin agonists of Formula (II) comprise one or more of the following integrin activating compounds:
(a) 3-oxo-1-(2-thienyl)-2-(2-thienylmethyl)-4-oxa-2,7-diaza-7-methyl-nonan-9-yl-bis(2-thienylmethyl)carbamate or 3,11-dioxo-1,13-bis(2-thienyl)-2,11-bis(2-thienylmethyl)-4,10-dioxa-2,6,12-triaza-7-methyl-tridecane,
(b) 3-oxo-1-(2-thienyl)-2-(2-thienylmethyl)-4,7,13-trioxa-2,10-diaza-10-methyl-pentadecan-15-yl-bis(2-thienylmethyl)carbamate,
(c) 3-oxo-1-(2-thienyl)-2-(2-thienylmethyl)-4,7,10,16,19-pentaoxa-2,13-diaza-13-methyl-heneicosan-21-yl-bis(2-thienylmethyl)carbamate,
(d) 3-oxo-1-(3-methoxyphenyl)-2-(3-methoxybenzyl)-4-oxa-2,7-diaza-7-methyl-nonan-9-yl-bis(3-methoxybenzyl)carbamate,
(e) 3-oxo-1-(3-methoxyphenyl)-2-(3-methoxybenzyl)-4,7,13-trioxa-2,10-diaza-10-methyl-pentadecan-15-yl-bis(3-methoxybenzyl)carbamate,
(f) 3-oxo-1-(3-methoxyphenyl)-2-(3-methoxybenzyl)-4,7,10,16,19-pentaoxa-2,13-diaza-13-methyl-heneicosan-21-yl-bis(3-methoxybenzyl)carbamate,
(g) 3-oxo-1-(3-methoxyphenyl)-2-(4-dimethylaminobenzyl)-4-oxa-2,7-diaza-7-methyl-nonan-9-yl-(3-methoxybenzyl)(4-dimethylaminobenzyl)carbamate,
(h) 3-oxo-1-(3-methoxyphenyl)-2-(4-dimethylaminobenzyl)-4,7,13-trioxa-2,10-diaza-10-methyl-pentadecan-15-yl-(3-methoxybenzyl)(4-dimethylaminobenzyl)carbamate,
(i) 3-oxo-1-(3-methoxyphenyl)-2-(4-dimethylaminobenzyl)-4,7,10,16,19-pentaoxa-2,13-diaza-13-methyl-heneicosan-2l-yl-(3-methoxybenzyl)(4-dimethylaminobenzyl)carbamate,
(j) 3-oxo-1-(4-methoxyphenyl)-2-(4-methoxybenzyl)-4-oxa-2,7-diaza-7-methyl-nonan-9-yl-bis(4-methoxybenzyl)carbamate,
(k) 3-oxo-1-(4-methoxyphenyl)-2-(4-methoxybenzyl)-4,7,13-trioxa-2,10-diaza-10-methyl-pentadecan-15-yl-bis(4-methoxybenzyl)carbamate,
(l) 3-oxo-1-(4-methoxyphenyl)-2-(4-methoxybenzyl)-4,7,10,16,19-pentaoxa-2,13-diaza-13-methyl-heneicosan-21-yl-bis(4-methoxybenzyl)carbamate,
(m) 3-oxo-1-(4-methoxyphenyl)-2-(4-dimethylaminobenzyl)-4-oxa-2,7-diaza-7-methyl-nonan-9-yl-(4-methoxybenzyl)(4-dimethylaminobenzyl) carbamate,
(n) 3-oxo-1-(4-methoxyphenyl)-2-(4-dimethylaminobenzyl)-4,7,13-trioxa-2,10-diaza-10-methyl-pentadecan-15-yl-(4-methoxybenzyl)(4-dimethylaminobenzyl)carbamate,
(o) 3-oxo-1-(4-methoxyphenyl)-2-(4-dimethylaminobenzyl)-4,7,10,16,19-pentaoxa-2,13-diaza-13-methyl-heneicosan-21-yl-(4-methoxybenzyl)(4-dimethylaminobenzyl)carbamate,
(p) 2-{[2-(9H-carbazol-9-ylcarbonyloxy)ethyl]-N-methylamino}ethyl 9H-carbazole-9-carboxylate,
(q) 2-[2-({2-[2-(9H-carbazol-9-ylcarbonyloxy)ethoxy]ethyl}-N-methylamino)ethoxy]ethyl 9H-carbazole-9-carboxylate,
(r) 2-(2-{2-[(2-{2-[2-(9-carbazolylcarbonyloxy)ethoxy]ethoxy}ethyl)-N-methylamino]ethoxy}ethoxy)ethyl 9-carbazolecarboxylate,
(s) 2-{[2-(3,6-dimethoxy-9H-carbazol-9-ylcarbonyloxy)ethyl]-N-methylamino}ethyl 3,6-dimethoxy-9H-carbazole-9-carboxylate,
(t) 2-[2-({2-[2-(3,6-dimethoxy-9H-carbazol-9-ylcarbonyloxy)ethoxy]ethyl}-N-methylamino) ethoxy]ethyl 3,6-dimethoxy-9H-carbazole-9-carboxylate,
(u) 2-(2-{2-[(2-{2-[2-(3,6-dimethoxy-9-carbazolylcarbonyloxy)ethoxy]ethoxy}ethyl)-N-methylamino]ethoxy}ethoxy)ethyl 3,6-dimethoxy-9-carbazolecarboxylate,
(v) higher analogs, or
(w) mixtures and combinations thereof.

### R^{a} and R^{b}═(OCH₂)ₙ or (CH₃)(CH₂O)ₙ and Z=2,6-Dihydroxy Pyridine Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and the Z group is derived from 2,6-dihydroxypyridine. In other embodiments, the integrin agonists of Formula (II) comprise one or more of the following integrin activating compounds:
(a) 2-[bis(thenyl)aminocarbonyloxy],6-[bis(thenyl)aminocarbonyloxy]pyridine,
(b) 2-[bis(3-methoxybenzyl)aminocarbonyloxy],6-[bis(3-methoxybenzyl)aminocarbonyloxy]pyridine,
(c) 2-[(3-methoxybenzyl)(4-dimethylaminobenzyl)aminocarbonyloxy], 6-[(3-methoxybenzyl)(4-dimethylaminobenzyl)aminocarbonyloxy]pyridine,
(d) 2-[bis(4-methoxybenzyl)aminocarbonyloxy], 6-[bis(4-methoxybenzyl)aminocarbonyloxy]pyridine,
(e) 2-[(4-methoxybenzyl)(4-dimethylaminobenzyl)aminocarbonyloxy], 6-[(4-methoxybenzyl)(4-dimethylaminobenzyl)aminocarbonyloxy]pyridine,
(f) 6-(9H-carbazol-9-ylcarbonyloxy)-2-pyridyl 9H-carbazole-9-carboxylate,
(g) 6-(3,6-dimethoxy-9H-carbazol-9-ylcarbonyloxy)-2-pyridyl 3,6-dimethoxy-9H-carbazole-9-carboxylate, or (h) mixtures and combinations thereof.

In other embodiments, the integrin activating compounds comprise one or more of the following integrin activating compounds:
(a) 2-[bis(thenyl)aminocarbonyloxymethoxy],6-[bis(thenyl)aminocarbonyloxy methoxy] pyridine,
(b) 2-[bis(thenyl)aminocarbonyloxymethoxymethoxy],6-[bis(thenyl)amino carbonyloxy methoxymethoxy]pyridine,
(c) 2-[bis(3-methoxybenzyl)aminocarbonyloxymethoxy],6-[bis(3-methoxybenzyl) amino carbonyloxymethoxy]pyridine,
(d) 2-[bis(3-methoxybenzyl)aminocarbonyloxymethoxymethoxy],6-[bis(3-methoxybenzyl)amino carbonyloxymethoxyemthyloxy] pyridine,
(e) 2-[(3-methoxybenzyl)(4-dimethylaminobenzyl)aminocarbonyloxymethoxy], 6-[(3-methoxybenzyl)(4-dimethylaminobenzyl)aminocarbonyloxy methoxy]pyridine,
(f) 2-[(3-methoxybenzyl)(4-dimethylaminobenzyl) aminocarbonyloxymethoxy methoxy],6-[(3-methoxybenzyl)(4-dimethylaminobenzyl)aminocarbonyloxy methoxymethoxy]pyridine,
(g) 2-[bis(4-methoxybenzyl)aminocarbonyloxymethoxy],6-[bis(4-methoxybenzyl)amino carbonyloxymethoxy]pyridine,
(h) 2-[bis(4-methoxybenzyl)aminocarbonyloxymethoxymethoxy],6-[bis(4-methoxybenzyl) aminocarbonyloxymethoxymethoxy] pyridine,
(i) 2-[(4-methoxybenzyl)(4-dimethylaminobenzyl)aminocarbonyloxymethoxy], 6-[(4-methoxybenzyl)(4-dimethylaminobenzyl)aminocarbonyloxymethoxy]pyridine,
(j) 2-[(4-methoxybenzyl)(4-dimethylaminobenzyl)aminocarbonyloxymethoxymethoxy],6-[(4-methoxybenzyl)(4-dimethylaminobenzyl)aminocarbonyloxymethoxymethoxy]pyridine,
(k) 2-{6-[2-(9H-carbazol-9-ylcarbonyloxy)methoxy]-2-pyridyloxy}methyl 9H-carbazole-9-carboxylate,
(l) 2-[2-(6-{2-[2-(9-carbazolylcarbonyloxy)methoxy]methoxy}-2-pyridyloxy)methoxy]methyl 9-carbazolecarboxylate,
(m) 2-{6-[2-(3,6-dimethoxy-9H-carbazol-9-ylcarbonyloxy)methoxy]-2-pyridyloxy}methyl 3,6-dimethoxy-9H-carbazole-9-carboxylate,
(n) 2-[2-(6-{2-[2-(3,6-dimethoxy-9-carbazolylcarbonyloxy)methoxy]methoxy}-2-pyridyloxy)methoxy]methyl 3,6-dimethoxy-9-carbazolecarboxylate,
(o) higher analogs, or
(p) mixtures and combinations thereof.

In other embodiments, the integrin activating compounds comprise one or more of the following integrin activating compounds:
(a) 2-[bis(thenyl)aminocarbonyloxyethoxy],6-[bis(thenyl)amino carbonyloxyethoxy]pyridine,
(b) 2-[bis(thenyl)aminocarbonyloxyethoxyethoxy],6-[bis(thenyl)aminocarbonyloxy ethoxyethoxy]pyridine,
(c) 2-[bis(3-methoxybenzyl)aminocarbonyloxyethoxy],6-[bis(3-methoxybenzyl)amino carbonyloxyethoxy]pyridine,
(d) 2-[bis(3-methoxybenzyl)aminocarbonyloxyethoxyethoxy],6-[bis(3-methoxybenzyl)amino carbonyloxyethoxyethoxy] pyridine,
(e) 2-[(3-methoxybenzyl)(4-dimethylaminobenzyl)aminocarbonyloxyethoxy], 6-[(3-methoxybenzyl)(4-dimethylaminobenzyl)aminocarbonyloxyethoxy]pyridine,
(f) 2-[(3-methoxybenzyl)(4-dimethylaminobenzyl)aminocarbonyloxyethoxyethoxy], 6-[(3-methoxybenzyl)(4-dimethylaminobenzyl)aminocarbonyloxyethoxyethoxy]pyridine,
(g) 2-[bis(4-methoxybenzyl)aminocarbonyloxyethoxy], 6-[bis(4-methoxybenzyl)aminocarbonyloxyethoxy]pyridine,
(h) 2-[bis(4-methoxybenzyl)aminocarbonyloxyethoxyethoxy], 6-[bis(4-methoxybenzyl)aminocarbonyloxyethoxyethoxy] pyridine,
(i) 2-[(4-methoxybenzyl)(4-dimethylaminobenzyl)aminocarbonyloxyethoxy], 6-[(4-methoxybenzyl)(4-dimethylaminobenzyl)aminocarbonyloxyethoxy]pyridine,
(j) 2-[(4-methoxybenzyl)(4-dimethylaminobenzyl)aminocarbonyloxyethoxyethoxy], 6-[(4-methoxybenzyl)(4-dimethylaminobenzyl)aminocarbonyloxyethoxyethoxy]pyridine,
(k) 2-{6-[2-(9H-carbazol-9-ylcarbonyloxy)ethoxy]-2-pyridyloxy}ethyl 9H-carbazole-9-carboxylate,
(l) 2-[2-(6-{2-[2-(9-carbazolylcarbonyloxy)ethoxy]ethoxy}-2-pyridyloxy)ethoxy]ethyl 9-carbazolecarboxylate,
(m) 2-{6-[2-(3,6-dimethoxy-9H-carbazol-9-ylcarbonyloxy)ethoxy]-2-pyridyloxy}ethyl 3,6-dimethoxy-9H-carbazole-9-carboxylate,
(n) 2-[2-(6-{2-[2-(3,6-dimethoxy-9-carbazolylcarbonyloxy)ethoxy]ethoxy}-2-pyridyloxy)ethoxy]ethyl 3,6-dimethoxy-9-carbazolecarboxylate,
(o) higher analogs, or
(p) mixtures and combinations thereof.

### R^{a} and R^{b}═(OCH₂)ₙ or CH₃)(CH₂O)ₙ and Z=2,6-Dimethanol Pyridine Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 2,6-dimethanolpyridine. In other embodiments, the integrin agonists of Formula (II) comprise one or more of the following integrin activating compounds:
(a) 2-({bis[(2-thienyl)methyl]aminocarbonyloxy}methyl)-6-({bis[(2-thienyl)methyl]aminocarbonyloxy}methyl)pyridine,
(b) 2-({bis(3-methoxybenzyl)aminocarbonyloxy}methyl)-6-({bis(3-methoxybenzyl) aminocarbonyloxy}methyl)pyridine,
(c) 2-(3-methoxybenzyl,4-dimethylaminobenzyl)aminocarbonyloxy}methyl)-6-(3-methoxybenzyl,4-dimethylamino benzyl)aminocarbonyloxy}methyl) pyridine,
(d) 2-({bis(4-methoxybenzyl)aminocarbonyloxy}methyl)-6-({bis(4-methoxybenzyl) aminocarbonyloxy} methyl)pyridine,
(e) 2-(4-methoxybenzyl,4-dimethylaminobenzyl)aminocarbonyloxy}methyl)-6-(4-methoxybenzyl,4-dimethylaminobenzyl) aminocarbonyloxy}methyl) pyridine,
(f) {6-[(9H-carbazol-9-ylcarbonyloxy)methyl]-2-pyridyl} methyl 9H-carbazole-9-carboxylate,
(g) {6-[(3,6-dimethoxy-9H-carbazol-9-ylcarbonyloxy)methyl]-2-pyridyl}methyl 3,6-dimethoxy-9H-carbazole-9-carboxylate, or
(h) mixtures and combinations thereof.

In other embodiments, the integrin activating compounds comprise one or more of the following integrin activating compounds:
(a) 2-{bis[(2-thienyl)methyl]aminocarbonyloxy}methoxy)methyl]-6-{bis[(2-thienyl)methyl]aminocarbonyloxy}methoxy)methyl]pyridine,
(b) 2-{bis[(2-thienyl)methyl]aminocarbonyloxy}methoxymethoxy)methyl]-6-{bis[(2-thienyl)methyl]aminocarbonyloxy}methoxymethoxy)methyl]pyridine,
(c) 2-{bis(3-methoxybenzyl)aminocarbonyloxy}methoxy)methyl]-6-{bis(3-methoxybenzyl)aminocarbonyloxy} methoxy)methyl]pyridine,
(d) 2-{bis(3-methoxybenzyl)aminocarbonyloxy}methoxymethoxy)methyl]-6-{bis(3-methoxybenzyl)aminocarbonyloxy}methoxymethoxy) methyl]pyridine,
(e) 2-(3-methoxybenzyl,4-dimethylaminobenzyl)aminocarbonyloxy}methoxy)methyl]-6-(3-methoxybenzyl,4-dimethylaminobenzyl)aminocarbonyloxy}methoxy)methyl]pyridine,
(f) 2-(3-methoxybenzyl,4-dimethylaminobenzyl)aminocarbonyloxy}methoxymethoxy)methyl]-6-(3-methoxybenzyl,4-dimethylaminobenzyl)aminocarbonyloxy}methoxyemthoxy) methyl]pyridine,
(g) 2-{bis(4-methoxybenzyl)aminocarbonyloxy}methoxy)methyl]-6-{bis(4-methoxybenzyl) aminocarbonyloxy}methoxy)methyl]pyridine,
(h) 2-{bis(4-methoxybenzyl)aminocarbonyloxy}methoxymethoxy)methyl]-6-{bis(4-methoxybenzyl)aminocarbonyloxy} methoxymethoxy)methyl]pyridine,
(i) 2-(4-methoxybenzyl,4-dimethylaminobenzyl)aminocarbonyloxy}methoxy)methyl]-6-(4-methoxybenzyl,4-dimethylaminobenzyl)amino carbonyloxy}methoxy)methyl]pyridine,
(j) 2-(4-methoxybenzyl,4-dimethylaminobenzyl)amino carbonyloxy}methoxyemthoxy)methyl]-6-(4-methoxybenzyl,4-dimethylaminobenzyl)amino carbonyloxy}methoxymethoxy)methyl]pyridine,
(k) 2-[(6-{[2-(9H-carbazol-9-ylcarbonyloxy)methoxy]methyl}-2-pyridyl)methoxy]ethyl 9H-carbazole-9-carboxylate,
(l) 2-(2-{[6-{2-[2-(9H-carbazolylcarbonyloxy)methoxy]methoxy}methyl)-2-pyridyl]methoxy}ethoxy)ethyl 9-carbazolecarboxylate,
(m) 2-[(6-{[2-(3,6-dimethoxy-9H-carbazol-9-ylcarbonyloxy)methoxy]methyl}-2-pyridyl) methoxy]ethyl 3,6-dimethoxy-9H-carbazole-9-carboxylate,
(n) 2-(2-{[6-{2-[2-(3,6-dimethoxy-9H-carbazolylcarbonyloxy)methoxy]methoxy}methyl)-2-pyridyl]methoxy}methoxy)ethyl 3,6-dimethoxy-9-carbazolecarboxylate,
(o) higher analogs, or
(p) mixtures and combinations thereof.

In other embodiments, the integrin activating compounds comprise one or more of the following integrin activating compounds:
(a) 2-{bis[(2-thienyl)methyl]aminocarbonyloxy}ethoxy)methyl]-6-{bis[(2-thienyl)methyl]aminocarbonyloxy}ethoxy)methyl]pyridine,
(b) 2-{bis[(2-thienyl)methyl]aminocarbonyloxy}ethoxyethoxy)methyl]-6-{bis[(2-thienyl)methyl]aminocarbonyloxy}ethoxyethoxy)methyl]pyridine,
(c) 2-{bis(3-methoxybenzyl)aminocarbonyloxy}ethoxy)methyl]-6-{bis(3-methoxybenzyl)amino carbonyloxy}ethoxy)methyl]pyridine,
(d) 2-{bis(3-methoxybenzyl)aminocarbonyloxy}ethoxyethoxy)methyl]-6-{bis(3-methoxybenzyl)aminocarbonyloxy}ethoxyethoxy)methyl]pyridine,
(e) 2-(3-methoxybenzyl,4-dimethylaminobenzyl)aminocarbonyloxy}ethoxy)methyl]-6-(3-methoxybenzyl,4-dimethylaminobenzyl)aminocarbonyloxy}ethoxy)methyl]pyridine,
(f) 2-(3-methoxybenzyl,4-dimethylaminobenzyl)aminocarbonyloxy}ethoxyethoxy)methyl]-6-(3-methoxybenzyl,4-dimethylaminobenzyl)aminocarbonyloxy}ethoxyethoxy)methyl]pyridine,
(g) 2-{bis(4-methoxybenzyl)aminocarbonyloxy}ethoxy)methyl]-6-{bis(4-methoxybenzyl) aminocarbonyloxy}ethoxy)methyl]pyridine,
(h) 2-{bis(4-methoxybenzyl)aminocarbonyloxy}ethoxyethoxy)methyl]-6-{bis(4-methoxybenzyl)aminocarbonyloxy}ethoxyethoxy)methyl]pyridine,
(i) 2-(4-methoxybenzyl,4-dimethylaminobenzyl)aminocarbonyloxy}ethoxy)methyl]-6-(4-methoxybenzyl,4-dimethylaminobenzyl)aminocarbonyloxy}ethoxy)methyl]pyridine,
(j) 2-(4-methoxybenzyl,4-dimethylaminobenzyl)aminocarbonyloxy}ethoxyethoxy)methyl]-6-(4-methoxybenzyl,4-dimethylaminobenzyl)aminocarbonyloxy}ethoxyethoxy)methyl]pyridine,
(k) 2-[(6-{[2-(9H-carbazol-9-ylcarbonyloxy)ethoxy]methyl}-2-pyridyl)methoxy]ethyl 9H-carbazole-9-carboxylate,
(l) 2-(2-{[6-({2-[2-(9H-carbazolylcarbonyloxy)ethoxy]ethoxy}methyl)-2-pyridyl]methoxy}ethoxy)ethyl 9-carbazolecarboxylate,
(m) 2-[(6-{[2-(3,6-dimethoxy-9H-carbazol-9-ylcarbonyloxy)ethoxy]methyl}-2-pyridyl)methoxy]ethyl 3,6-dimethoxy-9H-carbazole-9-carboxylate,
(n) 2-(2-{[6-({2-[2-(3,6-dimethoxy-9H-carbazolylcarbonyloxy)ethoxy]ethoxy}methyl)-2-pyridyl]methoxy}ethoxy)ethyl 3,6-dimethoxy-9-carbazolecarboxylate,
(o) higher analogs, or
(p) mixtures and combinations thereof.

### R^{a} and R^{b}═(OCH₂)ₙ or CH₃)(CH₂O)ₙ and Z=3,5-Dihydroxy Pyridine Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 3,5-dihydroxypyridine. In other embodiments, the integrin agonists of Formula (II) comprise one or more of the following integrin activating compounds:
(a) 3-[bis(thenyl)aminocarbonyloxy],5-[bis(thenyl)aminocarbonyloxy]pyridine,
(b) 3-[bis(3-methoxybenzyl)aminocarbonyloxy],5-[bis(3-methoxybenzyl)aminocarbonyloxy]pyridine,
(c) 3-[(3-methoxybenzyl)(4-dimethylaminobenzyl)aminocarbonyloxy],5-[(3-methoxybenzyl)(4-dimethylaminobenzyl)aminocarbonyloxy]pyridine,
(d) 3-[bis(4-methoxybenzyl)aminocarbonyloxy],5-[bis(4-methoxybenzyl)aminocarbonyloxy]pyridine,
(e) 3-[(4-methoxybenzyl)(4-dimethylaminobenzyl)aminocarbonyloxy],5-[(4-methoxybenzyl)(4-dimethylaminobenzyl)aminocarbonyloxy]pyridine,
(f) 5-(9H-carbazol-9-ylcarbonyloxy)-3-pyridyl 9H-carbazole-9-carboxylate,
(g) 5-(3,6-dimethoxy-9H-carbazol-9-ylcarbonyloxy)-3-pyridyl 3,6-dimethoxy-9H-carbazole-9-carboxylate, or
(h) mixtures and combinations thereof.

In other embodiments, the integrin activating compounds comprise one or more of the following integrin activating compounds:
(a) 3-[bis(thenyl)aminocarbonyloxymethoxy],5-[bis(thenyl)amino carbonyloxymethoxy]pyridine,
(b) 3-[bis(thenyl)aminocarbonyloxymethyoxymethoxy], 5-[bis(thenyl)aminocarbonyloxy methoxymethoxy]pyridine,
(c) 3-[bis(3-methoxybenzyl)aminocarbonyloxymethoxy],5-[bis(3-methoxybenzyl)aminocarbonyloxy methoxy]pyridine,
(d) 3-[bis(3-methoxybenzyl)aminocarbonyloxymethoxymethoxy],5-[bis(3-methoxybenzyl)aminocarbonyloxymethoxymethoxy]pyridine,
(e) 3-[(3-methoxybenzyl)(4-dimethylaminobenzyl)aminocarbonyloxymethoxy],5-[(3-methoxybenzyl)(4-dimethylaminobenzyl)aminocarbonyloxymethoxy]pyridine,
(f) 3-[(4-methoxybenzyl)(4-dimethylaminobenzyl)aminocarbonyloxymethoxymethoxy],5-[(4-methoxybenzyl)(4-dimethylaminobenzyl)aminocarbonyloxymethoxymethoxy]pyridine,
(g) 3-[bis(4-methoxybenzyl)aminocarbonyloxymethoxy],5-[bis(4-methoxybenzyl)aminocarbonyloxy methoxy]pyridine,
(h) 3-[bis(3-methoxybenzyl)aminocarbonyloxymethoxymethoxy],5-[bis(3-methoxybenzyl)aminocarbonyloxymethoxymethoxy]pyridine,
(i) 3-[(4-methoxybenzyl)(4-dimethylaminobenzyl)aminocarbonyloxymethoxy],5-[(4-methoxybenzyl)(4-dimethylaminobenzyl)aminocarbonyloxymethoxy]pyridine,
(j) 3-[(4-methoxybenzyl)(4-dimethylaminobenzyl)aminocarbonyloxymethoxymethoxy],5-[(4-methoxybenzyl)(4-dimethylaminobenzyl)aminocarbonyloxymethoxymethoxy]pyridine,
(k) 2-{5-[2-(9H-carbazol-9-ylcarbonyloxy)methoxy]-3-pyridyloxy}ethyl 9H-carbazole-9-carboxylate,
(l) 2-[2-(5-{2-[2-(9-carbazolylcarbonyloxy)methoxy]methoxy}-3-pyridyloxy)ethoxy]ethyl 9-carbazolecarboxylate,
(m) 2-{5-[2-(3,6-dimethoxy-9H-carbazol-9-ylcarbonyloxy)methoxy]-3-pyridyloxy}ethyl 3,6-dimethoxy-9H-carbazole-9-carboxylate,
(n) 2-[2-(5-{2-[2-(3,6-dimethoxy-9H-carbazolylcarbonyloxy)methoxy]methoxy}-3-pyridyloxy)ethoxy]ethyl 3,6-dimethoxy-9-carbazolecarboxylate,
(o) higher analogs, or
(p) mixtures and combinations thereof.

In other embodiments, the integrin activating compounds comprise one or more of the following integrin activating compounds:
(a) 3-[bis(thenyl)aminocarbonyloxyethoxy],5-[bis(thenyl)aminocarbonyloxy ethoxy]pyridine,
(b) 3-[bis(thenyl)aminocarbonyloxyethyoxyethoxy],5-[bis(thenyl) aminocarbonyloxyethyoxyethoxy]pyridine,
(c) 3-[bis(3-methoxybenzyl)amino carbonyloxyethoxy],5-[bis(3-methoxybenzyl)aminocarbonyloxyethoxy]pyridine,
(d) 3-[bis(3-methoxybenzyl)aminocarbonyloxyethoxyethoxy],5-[bis(3-methoxybenzyl)aminocarbonyloxy ethoxyethoxy]pyridine,
(e) 3-[(3-methoxybenzyl)(4-dimethylaminobenzyl)amino carbonyloxy],5-[(3-methoxybenzyl)(4-dimethylaminobenzyl)aminocarbonyloxy]pyridine,
(f) 3-[(3-methoxybenzyl)(4-dimethylaminobenzyl)aminocarbonyloxyethoxy],5-[(3-methoxybenzyl)(4-dimethylaminobenzyl)aminocarbonyloxyethoxy]pyridine,
(g) 3-[(4-methoxybenzyl)(4-dimethylaminobenzyl)aminocarbonyloxyethoxyethoxy],5-[(4-methoxybenzyl)(4-dimethylaminobenzyl)aminocarbonyloxyethoxyethoxy]pyridine,
(h) 3-[bis(4-methoxybenzyl)aminocarbonyloxyethoxy],5-[bis(4-methoxybenzyl)amino carbonyloxyethoxy]pyridine,
(i) 3-[bis(3-methoxybenzyl)aminocarbonyloxy ethoxyethoxy],5-[bis(3-methoxybenzyl)aminocarbonyloxyethoxyethoxy]pyridine,
(j) 3-[(4-methoxybenzyl)(4-dimethylaminobenzyl)aminocarbonyloxyethoxy],5-[(4-methoxybenzyl)(4-dimethylaminobenzyl)aminocarbonyloxyethoxy]pyridine,
(k) 3-[(4-methoxybenzyl)(4-dimethylaminobenzyl)aminocarbonyloxyethoxyethoxy],5-[(4-methoxybenzyl)(4-dimethylaminobenzyl)aminocarbonyloxyethoxyethoxy]pyridine,
(l) 2-{5-[2-(9H-carbazol-9-ylcarbonyloxy)ethoxy]-3-pyridyloxy}ethyl 9H-carbazole-9-carboxylate,
(m) 2-[2-(5-{2-[2-(9-carbazolylcarbonyloxy)ethoxy]ethoxy}-3-pyridyloxy)ethoxy]ethyl 9-carbazolecarboxylate,
(n) 2-{5-[2-(3,6-dimethoxy-9H-carbazol-9-ylcarbonyloxy)ethoxy]-3-pyridyloxy}ethyl 3,6-dimethoxy-9H-carbazole-9-carboxylate,
(o) 2-[2-(5-{2-[2-(3,6-dimethoxy-9H-carbazolylcarbonyloxy)ethoxy]ethoxy}-3-pyridyloxy)ethoxy]ethyl 3,6-dimethoxy-9-carbazolecarboxylate,
(p) higher analogs, or
(q) mixtures and combinations thereof.

### R^{a} and R^{b}═(OCH₂)ₙ or CH₃)(CH₂O)ₙ and Z=3,5-Dimethanol Pyridine Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 3,5-dimethanolpyridine. In other embodiments, the integrin agonists of Formula (II) comprise one or more of the following integrin activating compounds:
(a) 3-({bis[(2-thienyl)methyl]aminocarbonyloxy}methyl)-5-({bis[(2-thienyl)methyl]amino carbonyloxy}methyl)pyridine,
(b) 3-({bis(3-methoxybenzyl)aminocarbonyloxy}methyl)-5-({bis(3-methoxybenzyl)aminocarbonyloxy}methyl)pyridine,
(c) 3-(3-methoxybenzyl,4-dimethyl aminobenzyl)aminocarbonyloxy}methyl)-5-(3-methoxybenzyl,4-dimethylaminobenzyl) aminocarbonyloxy}methyl)pyridine,
(d) 3-({bis(4-methoxybenzyl)aminocarbonyloxy}methyl)-5-({bis(4-methoxybenzyl)aminocarbonyloxy}methyl)pyridine,
(e) 3-(4-methoxybenzyl,4-dimethylaminobenzyl)aminocarbonyloxy}methyl)-5-(4-methoxybenzyl,4-dimethylaminobenzyl) aminocarbonyloxy}methyl)pyridine,
(f) {5-[(9H-carbazol-9-ylcarbonyloxy)methyl]-3-pyridyl}methyl 9H-carbazole-9-carboxylate,
(g) {5-[(3,6-dimethoxy-9H-carbazol-9-ylcarbonyloxy)methyl]-3-pyridyl}methyl 3,6-dimethoxy-9H-carbazole-9-carboxylate, or
(h) mixtures and combinations thereof.

In other embodiments, the integrin activating compounds comprise one or more of the following integrin activating compounds:
(a) 3-{bis[(2-thienyl)methyl]aminocarbonyloxy} methoxy)methyl]-5-{bis[(2-thienyl)methyl]aminocarbonyloxy}methoxy)methyl]pyridine,
(b) 3-{bis[(2-thienyl)methyl]aminocarbonyloxy}methoxymethoxy)methyl]-5-{bis[(2-thienyl)methyl] aminocarbonyloxy}methoxymethoxy)methyl]pyridine,
(c) 3-{bis(3-methoxybenzyl)amino carbonyloxy}methoxy)methyl]-5-{bis(3-methoxybenzyl)aminocarbonyloxy}methoxy) methyl]pyridine,
(d) 3-{bis(3-methoxybenzyl)aminocarbonyloxy}methoxymethoxy)methyl]-5-{bis(3-methoxybenzyl)aminocarbonyloxy}methoxymethoxy)methyl]pyridine,
(e) 3-(3-methoxybenzyl,4-dimethylaminobenzyl)aminocarbonyloxy}methoxy)methyl]-5-(3-methoxybenzyl,4-dimethylaminobenzyl)aminocarbonyloxy}methoxy)methyl]pyridine,
(f) 3-(3-methoxybenzyl,4-dimethylaminobenzyl)aminocarbonyloxy}methoxymethoxy)methyl]-5-(3-methoxybenzyl,4-dimethylaminobenzyl)aminocarbonyloxy}methoxymethoxy)methyl]pyridine,
(g) 3-{bis(4-methoxybenzyl)aminocarbonyloxy}methoxy)methyl]-5-{bis(4-methoxybenzyl) aminocarbonyloxy}methoxy)methyl]pyridine,
(h) 3-{bis(4-methoxybenzyl)aminocarbonyloxy} methoxyethoxy)methyl]-5-{bis(4-methoxybenzyl)aminocarbonyloxy}methoxymethoxy) methyl]pyridine,
(i) 3-(4-methoxybenzyl,4-dimethylaminobenzyl)aminocarbonyloxy}methoxy)methyl]-5-(4-methoxybenzyl,4-dimethylaminobenzyl)aminocarbonyloxy}methoxy)methyl]pyridine,
(j) 3-(4-methoxybenzyl,4-dimethylaminobenzyl)aminocarbonyloxy}methoxymethoxy)methyl]-5-(4-methoxybenzyl,4-dimethylaminobenzyl)aminocarbonyloxy}methoxymethoxy)methyl]pyridine,
(k) 2-[(5-{[2-(9H-carbazol-9-ylcarbonyloxy)methoxy]methyl}-3-pyridyl)methoxy]ethyl 9H-carbazole-9-carboxylate, (1) 2-(2-{[5-(12-[2-(9-carbazolylcarbonyloxy) methoxy]methoxy}methyl)-3-pyridyl]methoxy}ethoxy)ethyl 9-carbazolecarboxylate,
(m) 2-[(5-{[2-(3,6-dimethoxy-9H-carbazol-9-ylcarbonyloxy)methoxy]methyl-3-pyridyl)methoxy]ethyl 3,6-dimethoxy-9H-carbazole-9-carboxylate,
(n) 2-(2-{[5-({2-[2-(3,6-dimethoxy-9-carbazolylcarbonyloxy)methoxy]methoxy}methyl)-3-pyridyl]methoxy}ethoxy)ethyl 3,6-dimethoxy-9-carbazolecarboxylate,
(o) higher analogs, or
(p) mixtures and combinations thereof.

In other embodiments, the integrin activating compounds comprise one or more of the following integrin activating compounds:
(a) 3-{bis[(2-thienyl)methyl]aminocarbonyloxy}ethoxy)methyl]-5-{bis[(2-thienyl)methyl]aminocarbonyloxy}ethoxy)methyl]pyridine,
(b) 3-{bis[(2-thienyl)methyl]aminocarbonyloxy}ethoxymethoxy)methyl]-5-{bis[(2-thienyl)methyl]aminocarbonyloxy} ethoxyethoxy)methyl]pyridine,
(c) 3-{bis(3-methoxybenzyl)aminocarbonyloxy}ethoxy)methyl]-5-{bis(3-methoxybenzyl)aminocarbonyloxy}ethoxy)methyl]pyridine,
(d) 3-{bis(3-methoxybenzyl)aminocarbonyloxy}ethoxyethoxy)methyl]-5-{bis(3-methoxybenzyl)aminocarbonyloxy} ethoxyethoxy)methyl]pyridine,
(e) 3-(3-methoxybenzyl,4-dimethylaminobenzyl)aminocarbonyloxy}ethoxy)methyl]-5-(3-methoxybenzyl,4-dimethylaminobenzyl)aminocarbonyloxy} ethoxy)methyl]pyridine,
(f) 3-(3-methoxybenzyl,4-dimethylaminobenzyl)aminocarbonyloxy} ethoxymethoxy)methyl]-5-(3-methoxybenzyl,4-dimethylaminobenzyl)aminocarbonyloxy} ethoxyethoxy)methyl]pyridine,
(g) 3-{bis(4-methoxybenzyl)aminocarbonyloxy}ethoxy)methyl]-5-{bis(4-methoxybenzyl)aminocarbonyloxy}ethoxy)methyl]pyridine,
(h) 3-{bis(4-methoxybenzyl)aminocarbonyloxy}ethoxyethoxy)methyl]-5-{bis(4-methoxybenzyl)aminocarbonyloxy}ethoxyethoxy)methyl]pyridine,
(i) 3-(4-methoxybenzyl,4-dimethylaminobenzyl)aminocarbonyloxy}ethoxy)methyl]-5-(4-methoxybenzyl,4-dimethylaminobenzyl)aminocarbonyloxy}ethoxy)methyl]pyridine,
(j) 3-(4-methoxybenzyl,4-dimethylaminobenzyl)aminocarbonyloxy}ethoxyethoxy)methyl]-5-(4-methoxybenzyl,4-dimethylaminobenzyl)aminocarbonyloxy} ethoxyethoxy)methyl]pyridine,
(k) 2-[(5-{[2-(9H-carbazol-9-ylcarbonyloxy)ethoxy]methyl}-3-pyridyl)methoxy]ethyl 9H-carbazole-9-carboxylate,
(l) 2-(2-{[5-({2-[2-(9-carbazolylcarbonyloxy)ethoxy]ethoxy}methyl)-3-pyridyl]methoxy}ethoxy)ethyl 9-carbazolecarboxylate,
(m) 2-[(5-{[2-(3,6-dimethoxy-9H-carbazol-9-ylcarbonyloxy)ethoxy]methyl}-3-pyridyl)methoxy]ethyl 3,6-dimethoxy-9H-carbazole-9-carboxylate,
(n) 2-(2-{[5-({2-[2-(3,6-dimethoxy-9-carbazolylcarbonyloxy)ethoxy]ethoxy}methyl)-3-pyridyl]methoxy}ethoxy)ethyl 3,6-dimethoxy-9-carbazolecarboxylate,
(o) higher analogs, or
(p) mixtures and combinations thereof.

### R^{a} and R^{b}═(OCH₂)ₙ or (CH₃)(CH₂O)ₙ and Z=3,5-Dihydroxy-Dimethyl Aniline Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 3,5-dihydroxy-dimethylaniline. In other embodiments, the integrin agonists of Formula (II) comprise one or more of the following integrin activating compounds:
(a) 3-[bis(thenyl)aminocarbonyloxy],5-[bis(thenyl)aminocarbonyloxy]dimethylamino benzene,
(b) 3-[bis(3-methoxybenzyl)aminocarbonyloxy],5-[bis(3-methoxybenzyl)aminocarbonyloxy]dimethylaminobenzene,
(c) 3-[(3-methoxybenzyl)(4-dimethylaminobenzyl)aminocarbonyloxy],5-[(3-methoxybenzyl)(4-dimethylaminobenzyl)aminocarbonyloxy]dimethylamino benzene,
(d) 3-[bis(4-methoxybenzyl)aminocarbonyloxy],5-[bis(4-methoxybenzyl)aminocarbonyloxy]dimethylamino benzene,
(e) 3-[(4-methoxybenzyl)(4-dimethylaminobenzyl)aminocarbonyloxy],5-[(4-methoxybenzyl)(4-dimethylaminobenzyl)aminocarbonyloxy]dimethylamino benzene,
(f) 3-(9H-carbazol-9-ylcarbonyloxy)-5-(dimethylamino)phenyl 9H-carbazole-9-carboxylate,
(g) 3-(3,6-dimethoxy-9H-carbazol-9-ylcarbonyloxy)-5-(dimethylamino)phenyl-3,6-dimethoxy-9H-carbazole-9-carboxylate, or
(h) mixtures and combinations thereof.

In other embodiments, the integrin activating compounds comprise one or more of the following integrin activating compounds:
(a) 3-[bis(thenyl)aminocarbonyloxymethoxy],5-[bis(thenyl)aminocarbonyloxymethoxy]dimethylaminobenzene,
(b) 3-[bis(thenyl)aminocarbonyloxymethoxymethoxy],5-[bis(thenyl)aminocarbonyloxy methoxymethoxy]dimethylaminobenzene,
(c) 3-[bis(3-methoxybenzyl)aminocarbonyloxymethoxy],5-[bis(3-methoxybenzyl)aminocarbonyloxy methoxy]dimethylaminobenzene,
(d) 3-[bis(3-methoxybenzyl)aminocarbonyloxymethoxymethoxy],5-[bis(3-methoxybenzyl)amino carbonyloxymethoxymethoxy] dimethylaminobenzene,
(e) 3-[(3-methoxybenzyl)(4-dimethylaminobenzyl)aminocarbonyloxymethoxy],5-[(3-methoxy benzyl)(4-dimethylaminobenzyl)aminocarbonyloxy methoxy]dimethylaminobenzene,
(f) 3-[(3-methoxybenzyl)(4-dimethylaminobenzyl)aminocarbonyloxymethoxymethoxy],5-[(3-methoxybenzyl)(4-dimethylaminobenzyl)aminocarbonyloxymethoxymethoxy]dimethylamino benzene,
(g) 3-[bis(4-methoxybenzyl)aminocarbonyloxymethoxy],5-[bis(4-methoxybenzyl)amino carbonyloxymethoxy]dimethylaminobenzene,
(h) 3-[bis(4-methoxybenzyl)aminocarbonyloxymethoxymethoxy],5-[bis(4-methoxybenzyl) aminocarbonyloxymethoxymethoxy] dimethylaminobenzene,
(i) 3-[(4-methoxybenzyl)(4-dimethylaminobenzyl)aminocarbonyloxymethoxy],5-[(4-methoxybenzyl)(4-dimethylaminobenzyl)aminocarbonyloxymethoxy] dimethylaminobenzene,
(j) 3-[(4-methoxybenzyl)(4-dimethylaminobenzyl)aminocarbonyloxymethoxymethoxy],5-[(4-methoxybenzyl)(4-dimethylaminobenzyl)aminocarbonyloxymethoxymethoxy]dimethylamino benzene,
(k) 2-{3-[2-(9H-carbazol-9-ylcarbonyloxy)methoxy]-5-(dimethylamino)phenoxy}methyl 9H-carbazole-9-carboxylate,
(l) 2-[2-(3-{2-[2-(9-carbazolylcarbonyloxy)methoxy]methoxy}-5-(dimethylamino)phenoxy) methoxy]ethyl 9-carbazolecarboxylate,
(m) 2-{3-[2-(3,6-dimethoxy-9H-carbazol-9-ylcarbonyloxy)methoxy]-5-(dimethylamino)phenoxy} ethyl 3,6-dimethoxy-9H-carbazole-9-carboxylate,
(n) 2-[2-(3-{2-[2-(3,6-dimethoxy-9-carbazolylcarbonyloxy)methoxy]methoxy}-5-(dimethylamino) phenoxy)methoxy]methyl 3,6-dimethoxy-9-carbazolecarboxylate,
(o) higher analogs, or
(p) mixtures and combinations thereof.

In other embodiments, the integrin activating compounds comprise one or more of the following integrin activating compounds:
(a) 3-[bis(thenyl)aminocarbonyloxyethoxy],5-[bis(thenyl)aminocarbonyloxyethoxy]dimethylamino benzene,
(b) 3-[bis(thenyl)aminocarbonyloxyethoxyethoxy],5-[bis(thenyl)amino carbonyloxyethoxyethoxy] dimethylaminobenzene,
(c) 3-[bis(3-methoxybenzyl) aminocarbonyloxyethoxy],5-[bis(3-methoxybenzyl)aminocarbonyloxy ethoxy]dimethylaminobenzene,
(d) 3-[bis(3-methoxybenzyl)aminocarbonyloxyethoxyethoxy],5-[bis(3-methoxybenzyl)amino carbonyloxyethoxyethoxy]dimethylaminobenzene,
(e) 3-[(3-methoxybenzyl)(4-dimethylaminobenzyl)aminocarbonyloxyethoxy],5-[(3-methoxybenzyl)(4-dimethylaminobenzyl)aminocarbonyloxyethoxy] dimethylaminobenzene,
(f) 3-[(3-methoxybenzyl)(4-dimethylaminobenzyl)aminocarbonyloxyethoxyethoxy],5-[(3-methoxybenzyl)(4-dimethylaminobenzyl)aminocarbonyloxyethoxyethoxy]dimethylaminobenzene,
(g) 3-[bis(4-methoxybenzyl)aminocarbonyloxyethoxy],5-[bis(4-methoxybenzyl)aminocarbonyloxy ethoxy]dimethylaminobenzene,
(h) 3-[bis(4-methoxybenzyl)aminocarbonyloxyethoxyethoxy],5-[bis(4-methoxybenzyl)amino carbonyloxyethoxyethoxy]dimethylaminobenzene,
(i) 3-[(4-methoxybenzyl)(4-dimethylaminobenzyl)aminocarbonyloxyethoxy],5-[(4-methoxybenzyl)(4-dimethylaminobenzyl)aminocarbonyloxyethoxy]dimethylaminobenzene,
(j) 3-[(4-methoxybenzyl)(4-dimethylaminobenzyl)aminocarbonyloxyethoxyethoxy],5-[(4-methoxybenzyl)(4-dimethylaminobenzyl)aminocarbonyloxyethoxyethoxy]dimethylaminobenzene,
(k) 2-{3-[2-(9H-carbazol-9-ylcarbonyloxy)ethoxy]-5-(dimethylamino)phenoxy}ethyl 9H-carbazole-9-carboxylate,
(l) 2-[2-(3-{2-[2-(9-carbazolylcarbonyloxy)ethoxy]ethoxy}-5-(dimethylamino)phenoxy)ethoxy]ethyl 9-carbazolecarboxylate,
(m) 2-{3-[2-(3,6-dimethoxy-9H-carbazol-9-ylcarbonyloxy)ethoxy]-5-(dimethylamino)phenoxy}ethyl 3,6-dimethoxy-9H-carbazole-9-carboxylate,
(n) 2-[2-(3-{2-[2-(3,6-dimethoxy-9-carbazolylcarbonyloxy)ethoxy]ethoxy}-5-(dimethylamino) phenoxy)ethoxy]ethyl 3,6-dimethoxy-9-carbazolecarboxylate,
(o) higher analogs, or
(p) mixtures and combinations thereof.

### R^{a} and R^{b}═(OCH₂)ₙ or CH₃)(CH₂O)ₙ and Z=3,5-Dihydroxy-Dimethyl Benzyl Amine Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 3,5-dihydroxy-Dimethyl-BenzylAmine. In other embodiments, the integrin activating compounds comprise one or more of the following integrin activating compounds:
(a) 3-[bis(thenyl)aminocarbonyloxy],5-[bis(thenyl)aminocarbonyloxy] dimethylamino benzylamine,
(b) 3-[bis(3-methoxybenzyl)aminocarbonyloxy],5-[bis(3-methoxybenzyl)amino carbonyloxy]dimethylamino benzylamine,
(c) 3-[(3-methoxybenzyl)(4-dimethylaminobenzyl)aminocarbonyloxy],5-[(3-methoxybenzyl)(4-dimethylaminobenzyl)amino carbonyloxy]dimethylamino benzylamine,
(d) 3-[bis(4-methoxybenzyl)aminocarbonyloxy],5-[bis(4-methoxybenzyl)amino carbonyloxy]dimethylaminobenzylamine,
(e) 3-[(4-methoxybenzyl)(4-dimethylaminobenzyl) aminocarbonyloxy],5-[(4-methoxybenzyl)(4-dimethylaminobenzyl)aminocarbonyloxy] dimethylaminobenzylamine,
(f) 3-(9H-carbazol-9-ylcarbonyloxy)-5-[(methylamino)methyl]phenyl 9H-carbazole-9-carboxylate,
(g) 3-(3,6-dimethoxy-9H-carbazol-9-ylcarbonyloxy)-5-[(dimethylamino)methyl]phenyl 3,6-dimethoxy-9H-carbazole-9-carboxylate, or
(h) mixtures and combinations thereof.

In other embodiments, the integrin activating compounds comprise one or more of the following integrin activating compounds:
(a) 3-[bis(thenyl)aminocarbonyloxymethoxy],5-[bis(thenyl)aminocarbonyloxymethoxy]dimethylaminobenzylamine,
(b) 3-[bis(thenyl)aminocarbonyloxymethoxymethoxy],5-[bis(thenyl)aminocarbonyloxymethoxy methoxy]dimethylaminobenzylamine,
(c) 3-[bis(3-methoxybenzyl)aminocarbonyloxymethoxy],5-[bis(3-methoxybenzyl)aminocarbonyloxy methoxy]dimethylaminobenzylamine,
(d) 3-[bis(3-methoxybenzyl)amino carbonyloxymethoxymethoxy],5-[bis(3-methoxybenzyl)amino carbonyloxymethoxymethoxy] dimethylaminobenzylamine,
(e) 3-[(3-methoxybenzyl)(4-dimethylaminobenzyl)aminocarbonyloxymethoxy],5-[(3-methoxy benzyl)(4-dimethylaminobenzyl)aminocarbonyloxymethoxy] dimethylaminobenzylamine,
(f) 3-[(3-methoxybenzyl)(4-dimethylaminobenzyl)aminocarbonyloxy methoxymethoxy],5-[(3-methoxybenzyl)(4-dimethylaminobenzyl)aminocarbonyloxymethoxymethoxy],dimethylaminobenzyl amine,
(g) 3-[bis(4-methoxybenzyl)aminocarbonyloxymethoxy],5-[bis(4-methoxybenzyl)aminocarbonyloxy methoxy]dimethylaminobenzylamine,
(h) 3-[bis(4-methoxybenzyl)aminocarbonyloxymethoxymethoxy],5-[bis(4-methoxybenzyl) aminocarbonyloxyethoxymethoxy]dimethylaminobenzylamine,
(i) 3-[(4-methoxybenzyl)(4-dimethylaminobenzyl)aminocarbonyloxymethoxy],5-[(4-methoxybenzyl)(4-dimethylaminobenzyl)aminocarbonyloxymethoxy]dimethylaminobenzylamine,
(j) 3-[(4-methoxybenzyl)(4-dimethylaminobenzyl)aminocarbonyloxymethoxymethoxy],5-[(4-methoxybenzyl)(4-dimethylaminobenzyl)aminocarbonyloxymethoxymethoxy]dimethylamino benzylamine,
(k) 2-{3-[2-(9H-carbazol-9-ylcarbonyloxy)methoxy]-5-[(dimethylamino)methyl]phenoxy}methyl 9H-carbazole-9-carboxylate,
(l) 2-[2-(3-{2-[2-(9-carbazolylcarbonyloxy)methoxy]methoxy}-5-[(dimethylamino)methyl]phenoxy) methoxy]methyl 9-carbazolecarboxylate,
(m) 2-{3-[2-(3,6-dimethoxy-9H-carbazol-9-ylcarbonyloxy)ethoxy]-5-[(dimethylamino)methyl]phenoxy}methyl 3,6-dimethoxy-9H-carbazole-9-carboxylate,
(n) 2-[2-(3-{2-[2-(3,6-dimethoxy-9-carbazolylcarbonyloxy)methoxy]methoxy}-5-[(dimethylamino) methyl] phenoxy)methoxy]methyl 3,6-dimethoxy-9-carbazolecarboxylate,
(o) higher analogs, or
(p) mixtures and combinations thereof.

In other embodiments, the integrin activating compounds comprise one or more of the following integrin activating compounds:
(a) 3-[bis(thenyl)aminocarbonyloxyethoxy],5-[bis(thenyl)aminocarbonyloxyethoxy]dimethylamino benzylamine,
(b) 3-[bis(thenyl)aminocarbonyloxyethoxyethoxy], 5-[bis(thenyl)aminocarbonyloxyethoxyethoxy] dimethylaminobenzylamine,
(c) 3-[bis(3-methoxybenzyl)aminocarbonyloxyethoxy],5-[bis(3-methoxybenzyl)aminocarbonyloxy ethoxy]dimethylaminobenzylamine,
(d) 3-[bis(3-methoxybenzyl)aminocarbonyloxyethoxyethoxy],5-[bis(3-methoxybenzyl)amino carbonyloxyethoxyethoxy]dimethylaminobenzylamine,
(e) 3-[(3-methoxybenzyl)(4-dimethylaminobenzyl)aminocarbonyloxyethoxy],5-[(3-methoxybenzyl)(4-dimethylaminobenzyl)aminocarbonyloxyethoxy]dimethylaminobenzylamine,
(f) 3-[(3-methoxybenzyl)(4-dimethylaminobenzyl)aminocarbonyloxyethoxyethoxy],5-[(3-methoxybenzyl)(4-dimethylaminobenzyl)aminocarbonyloxyethoxyethoxy], dimethylaminobenzyl amine,
(g) 3-[bis(4-methoxybenzyl)aminocarbonyloxyethoxy],5-[bis(4-methoxybenzyl)aminocarbonyloxy ethoxy]dimethylaminobenzylamine,
(h) 3-[bis(4-methoxybenzyl)aminocarbonyloxyethoxyethoxy],5-[bis(4-methoxybenzyl)amino carbonyloxyethoxyethoxy]dimethylaminobenzylamine,
(i) 3-[(4-methoxybenzyl)(4-dimethylaminobenzyl)aminocarbonyloxyethoxy],5-[(4-methoxy benzyl)(4-dimethylaminobenzyl)aminocarbonyloxyethoxy]dimethylaminobenzylamine,
(j) 3-[(4-methoxybenzyl)(4-dimethylaminobenzyl)aminocarbonyloxyethoxyethoxy],5-[(4-methoxybenzyl)(4-dimethylaminobenzyl)aminocarbonyloxyethoxyethoxy]dimethylamino benzylamine,
(k) 2-{3-[2-(9H-carbazol-9-ylcarbonyloxy)ethoxy]-5-[(dimethylamino)methyl]phenoxy}ethyl 9H-carbazole-9-carboxylate,
(l) 2-[2-(3-{2-[2-(9-carbazolylcarbonyloxy)ethoxy]ethoxy}-5-[(dimethylamino)methyl]phenoxy) ethoxy]ethyl 9-carbazolecarboxylate,
(m) 2-{3-[2-(3,6-dimethoxy-9H-carbazol-9-ylcarbonyloxy)ethoxy]-5-[(dimethylamino)methyl]phenoxy}ethyl 3,6-dimethoxy-9H-carbazole-9-carboxylate,
(n) 2-[2-(3-{2-[2-(3,6-dimethoxy-9-carbazolylcarbonyloxy)ethoxy]ethoxy}-5-[(dimethylamino) methyl]phenoxy)ethoxy]ethyl 3,6-dimethoxy-9-carbazolecarboxylate,
(o) higher analogs, or
(p) mixtures and combinations thereof.

### R^{a} and R¹═(OCH₂)ₙ or CH₃)(CH₂O)ₙ and Z=2,6-Pyridine Dicarboxylic Acid Chloride Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 2,6-pyridine dicarboxylic acid chloride. In other embodiments, the integrin agonists of Formula (II) comprise one or more of the following integrin activating compounds:
(a) N,N,N,N-tetra(2-thienylmethyl)-2,6-pyridinedicarboxamide,
(b) N,N,N,N-tetra(3-methoxybenzyl)-2,6-pyridinedicarboxamide,
(c) N,N-bis(3-methoxybenzyl)-N,N-bis(4-dimethylaminobenzyl)-2,6-pyridinedicarboxamide,
(d) N,N,N,N-tetra(4-methoxybenzyl)-2,6-pyridinedicarboxamide,
(e) N,N-bis(4-methoxybenzyl)-N,N-bis(4-dimethylaminobenzyl)-2,6-pyridinedicarboxamide,
(f) or mixtures and combinations thereof.

In other embodiments, the integrin activating compounds comprise one or more of the following integrin activating compounds:
(a) bis(2-{bis(2-thienylmethyl)aminocarbonyloxy}methyl)2,6-pyridinedicarboxylate,
(b) bis(2-{bis(2-thienylmethyl)aminocarbonyloxymethoxy}methyl)2,6-pyridinedicarboxylate,
(c) bis(2-{bis[3-methoxybenzyl]aminocarbonyloxy}methyl)2,6-pyridinedicarboxylate,
(d) bis(2-{bis[3-methoxybenzyl]aminocarbonyloxymethoxy}methyl)2,6-pyridinedicarboxylate,
(e) bis(2-{(3-methoxybenzyl)(4-dimethylaminobenzyl)aminocarbonyloxy}methyl) 2,6-pyridinedicarboxylate,
(f) bis(2-{(4-methoxybenzyl)(4-dimethylaminobenzyl)aminocarbonyloxy methoxy}methyl) 2,6-pyridinedicarboxylate,
(g) bis(2-{bis[4-methoxybenzyl]aminocarbonyloxy}methyl)2,6-pyridinedicarboxylate,
(h) bis(2-{bis[4-methoxybenzyl]aminocarbonyloxymethoxy} methyl)2,6-pyridinedicarboxylate,
(i) bis(2-{(4-methoxybenzyl)(4-dimethylaminobenzyl) aminocarbonyloxy}methyl) 2,6-pyridinedicarboxylate,
(j) bis(2-{(4-methoxybenzyl)(4-dimethylaminobenzyl)aminocarbonyloxymethoxy} methyl)2,6-pyridinedicarboxylate,
(k) higher analogs, or
(l) mixtures and combinations thereof.

In other embodiments, the integrin activating compounds comprise one or more of the following integrin activating compounds:
(a) bis(2-{bis(2-thienylmethyl)aminocarbonyloxy}ethyl)2,6-pyridinedicarboxylate,
(b) bis(2-{bis(2-thienylmethyl)aminocarbonyloxyethoxy}ethyl)2,6-pyridinedicarboxylate,
(c) bis(2-{bis[3-methoxybenzyl]aminocarbonyloxy}ethyl)2,6-pyridinedicarboxylate,
(d) bis(2-{bis[3-methoxybenzyl]aminocarbonyloxyethoxy}ethyl)2,6-pyridinedicarboxylate,
(e) bis(2-{(3-methoxybenzyl)(4-dimethylaminobenzyl)aminocarbonyloxy}ethyl)2,6-pyridine dicarboxylate,
(f) bis(2-{(4-methoxybenzyl)(4-dimethylaminobenzyl)aminocarbonyloxyethoxy}ethyl) 2,6-pyridinedicarboxylate,
(g) bis(2-{bis[4-methoxybenzyl]amino carbonyloxy}ethyl)2,6-pyridinedicarboxylate,
(h) bis(2-{bis[4-methoxybenzyl]aminocarbonyloxy ethoxy}ethyl)2,6-pyridinedicarboxylate,
(i) bis(2-{(4-methoxybenzyl)(4-dimethylaminobenzyl)aminocarbonyloxy}ethyl) 2,6-pyridinedicarboxylate,
(j) bis(2-{(4-methoxybenzyl)(4-dimethylaminobenzyl)aminocarbonyloxyethoxy}ethyl) 2,6-pyridinedicarboxylate,
(k) higher analogs, or
(l) mixtures and combinations thereof.

### R^{a} and R^{b}═(OCH₂)ₙ or CH₃)(CH₂O)ₙ and Z=3,5-Pyridine Dicarboxylic Acid Chloride Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 3,5-pyridine diecarboxylic acid chloride. In other embodiments, the integrin agonists of Formula (II) comprise one or more of the following integrin activating compounds:
(a) N,N,N,N-tetra(2-thienylmethyl)-3,5-pyridinedicarboxamide,
(b) N,N,N,N-tetra(4-methoxybenzyl)-3,5-pyridinedicarboxamide,
(c) N,N-bis(3-methoxybenzyl)-N,N-bis(4-dimethylaminobenzyl)-3,5-pyridinedicarboxamide,
(d) N,N,N,N-tetra(4-methoxybenzyl)-3,5-pyridinedicarboxamide,
(e) N,N-bis(4-methoxybenzyl)-N,N-bis(4-dimethylaminobenzyl)-3,5-pyridinedicarboxamide, or
(f) mixture and combinations thereof.

In other embodiments, the integrin activating compounds comprise one or more of the following integrin activating compounds:
(a) bis(2-{bis(2-thienylmethyl]aminocarbonyloxy}methyl)3,5-pyridinedicarboxylate,
(b) bis(2-{bis(2-thienylmethyl]aminocarbonyloxymethoxy}methyl)3,5-pyridinedicarboxylate,
(c) bis(2-{bis(3-methoxybenzyl)aminocarbonyloxy}methyl)3,5-pyridinedicarboxylate,
(d) bis(2-{bis[3-methoxybenzyl]aminocarbonyloxymethoxy}methyl)3,5-pyridinedicarboxylate,
(e) bis(2-{(3-methoxybenzyl)(4-methylaminobenzyl)aminocarbonyloxy}methyl) 3,5-pyridinedicarboxylate,
(f) bis(2-{(3-methoxybenzyl)(4-dimethylaminobenzyl)aminocarbonyloxymethoxy}methyl) 3,5-pyridinedicarboxylate,
(g) bis(2-{bis(4-methoxybenzyl)aminocarbonyloxy}methyl)3,5-pyridinedicarboxylate,
(h) bis(2-{bis[4-methoxybenzyl]aminocarbonyloxymethoxy}methyl)3,5-pyridinedicarboxylate,
(i) bis(2-{bis(4-methoxybenzyl)(4-methylaminobenzyl)aminocarbonyloxy}methyl) 3,5-pyridinedicarboxylate,
(j) bis(2-{(4-methoxybenzyl)(4-dimethylaminobenzyl)aminocarbonyloxymethoxy}methyl) 3,5-pyridinedicarboxylate,
(k) higher analogs, or
(l) mixtures and combinations thereof.

In other embodiments, the integrin activating compounds comprise one or more of the following integrin activating compounds:
(a) bis(2-{bis(2-thienylmethyl]aminocarbonyloxy}ethyl)3,5-pyridinedicarboxylate,
(b) bis(2-{bis(2-thienylmethyl]aminocarbonyloxyethoxy}ethyl)3,5-pyridinedicarboxylate,
(c) bis(2-{bis(3-methoxybenzyl)aminocarbonyloxy}ethyl)3,5-pyridinedicarboxylate,
(d) bis(2-{bis[3-methoxybenzyl]aminocarbonyloxyethoxy}ethyl)2,6-pyridinedicarboxylate,
(e) bis(2-{(3-methoxybenzyl)(4-methylaminobenzyl)aminocarbonyloxy}ethyl) 3,5-pyridine dicarboxylate,
(f) bis(2-{(3-methoxybenzyl)(4-dimethylaminobenzyl)aminocarbonyloxyethoxy}ethyl) 2,6-pyridine dicarboxylate,
(g) bis(2-{bis(4-methoxybenzyl)aminocarbonyloxy}ethyl)3,5-pyridinedicarboxylate,
(h) bis(2-{bis[4-methoxybenzyl]aminocarbonyloxyethoxy}ethyl)3,5-pyridinedicarboxylate,
(i) bis(2-{bis(4-methoxybenzyl)(4-methylaminobenzyl)aminocarbonyloxy}ethyl) 3,5-pyridine dicarboxylate,
(j) bis(2-{(4-methoxybenzyl)(4-dimethylaminobenzyl)aminocarbonyloxyethoxy}ethyl) 2,6-pyridine dicarboxylate,
(k) higher analogs, or
(l) mixtures and combinations thereof.

### R^{a} and R^{b}═(OCH₂)ₙ or CH₃)(CH₂O)ₙ and Z=3-Dimethylamino-1,5-Pentane Diol Class

In certain embodiments, the integrin agonists of Formula (II) comprise one or more integrin agonists, wherein the R^{a} and R^{b} group comprises (OCH₂)ₙ or (OCH₂CH₂)ₙ, wherein n is an integer between 1 and 6 and Z is derived from 3-dimethylamino-1,5-pentane diol. In other embodiments, the integrin agonists of Formula (II) comprise one or more of the following integrin activating compounds:
(a) 1-[bis(2-thienylmethyl)aminocarbonyloxy]-5-[bis(2-thienylmethyl)aminocarbonyloxy]-3-(dimethylamino)pentane,
(b) 1-[bis(3-methoxybenzyl)aminocarbonyloxy]-5-[bis(3-methoxybenzyl)aminocarbonyloxy]-3-(dimethylamino)pentane,
(c) 1-[(3-methoxybenzyl),(4-dimethylaminobenzyl) aminocarbonyloxy]-5-[bis(3-methoxybenzyl),(4-dimethylaminobenzyl) aminocarbonyloxy]-3-(dimethylamino)pentane,
(d) 1-[bis(4-methoxybenzyl)aminocarbonyloxy]-5-[bis(4-methoxybenzyl)aminocarbonyloxy]-3-(dimethylamino)pentane,
(e) 1-[(4-methoxybenzyl),(4-dimethylaminobenzyl) aminocarbonyloxy]-5-[bis(4-methoxybenzyl),(4-dimethylaminobenzyl) aminocarbonyloxy]-3-(dimethylamino)pentane, or
(f) mixtures and combinations thereof.

In other embodiments, the integrin activating compounds comprise one or more of the following integrin activating compounds:
(a) 1-[bis(2-thienylmethyl)aminocarbonyloxymethoxy]-5-[bis(2-thienylmethyl)aminocarbonyloxy methoxy]-3-(dimethylamino)pentane,
(b) 1-[bis(2-thienylmethyl)aminocarbonyloxymethoxymethoxy]-5-[bis(2-thienylmethyl)amino carbonyloxymethoxymethoxy]-3-(dimethylamino)pentane,
(c) 1-[bis(3-methoxybenzyl)aminocarbonyloxymethoxy]-5-[bis(3-methoxybenzyl)aminocarbonyloxy methoxy]-3-(dimethylamino)pentane,
(d) 1-[bis(3-methoxybenzyl)aminocarbonyloxymethoxymethoxy]-5-[bis(3-methoxybenzyl)amino carbonyloxymethoxymethoxy]-3-(dimethylamino)pentane,
(e) 1-{[(3-methoxybenzyl)(4-dimethylaminobenzyl)amino]carbonyloxymethoxy}-5-{[(3-methoxybenzyl)(4-dimethylaminobenzyl) amino]carbonyloxymethoxy}-3-(dimethylamino)pentane,
(f) 1-{[(3-methoxybenzyl)(4-dimethylaminobenzyl) amino]carbonyloxymethoxymethoxy}-5-{[(3-methoxybenzyl)(4-dimethylaminobenzyl)amino]carbonyloxymethoxymethoxy}-3-(dimethylamino)pentane,
(g) 1-[bis(4-methoxybenzyl)aminocarbonyloxymethoxy]-5-[bis(4-methoxybenzyl)amino carbonyloxy methoxy]-3-(dimethylamino)pentane,
(h) 1-[bis(4-methoxybenzyl)aminocarbonyloxymethoxymethoxy]-5-[bis(4-methoxybenzyl)amino carbonyloxymethoxymethoxy]-3-(dimethylamino)pentane,
(i) 1-{[(4-methoxybenzyl)(4-dimethylaminobenzyl)amino]carbonyloxymethoxy}-5-{[(4-methoxy benzyl)(4-dimethylaminobenzyl) amino]carbonyloxymethoxy}-3-(dimethylamino)pentane,
(j) 1-{[(4-methoxybenzyl)(4-dimethylaminobenzyl) amino]carbonyloxymethoxymethoxy}-5-{[(4-methoxybenzyl)(4-dimethylaminobenzyl)amino]carbonyloxymethoxymethoxy}-3-(dimethylamino)pentane,
(k) higher analogs, or
(l) mixtures and combinations thereof.

In other embodiments, the integrin activating compounds comprise one or more of the following integrin activating compounds:
(a) 1-[bis(2-thienylmethyl)aminocarbonyloxyethoxy]-5-[bis(2-thienylmethyl)aminocarbonyloxy ethoxy]-3-(dimethylamino)pentane,
(b) 1-[bis(2-thienylmethyl)aminocarbonyloxyethoxyethoxy]-5-[bis(2-thienylmethyl)amino carbonyloxyethoxyethoxy]-3-(dimethylamino)pentane,
(c) 1-[bis(3-methoxybenzyl)aminocarbonyloxyethoxy]-5-[bis(3-methoxybenzyl)aminocarbonyloxy ethoxy]-3-(dimethylamino)pentane,
(d) 1-[bis(3-methoxybenzyl)aminocarbonyloxyethoxyethoxy]-5-[bis(3-methoxybenzyl)amino carbonyloxyethoxyethoxy]-3-(dimethylamino)pentane,
(e) 1-{[(3-methoxybenzyl)(4-dimethylaminobenzyl)amino]carbonyloxyethoxy}-5-{[(3-methoxybenzyl)(4-dimethylaminobenzyl) amino]carbonyloxyethoxy}-3-(dimethylamino)pentane,
(f) 1-{[(3-methoxybenzyl)(4-dimethylaminobenzyl)amino]carbonyloxyethoxyethoxy}-5-{[(3-methoxybenzyl)(4-dimethylaminobenzyl)amino]carbonyloxyethoxyethoxy}-3-(dimethylamino) pentane,
(g) 1-[bis(4-methoxybenzyl)aminocarbonyloxyethoxy]-5-[bis(4-methoxybenzyl)aminocarbonyloxy ethoxy]-3-(dimethylamino)pentane,
(h) 1-[bis(4-methoxybenzyl)aminocarbonyloxyethoxyethoxy]-5-[bis(4-methoxybenzyl) aminocarbonyloxy ethoxyethoxy]-3-(dimethylamino)pentane,
(i) 1-{[(4-methoxybenzyl)(4-dimethylaminobenzyl)amino]carbonyloxyethoxy}-5-{[(4-methoxybenzyl)(4-dimethylaminobenzyl) amino]carbonyloxyethoxy}-3-(dimethylamino)pentane,
(j) 1-{[(4-methoxybenzyl)(4-dimethylaminobenzyl)amino]carbonyloxyethoxyethoxy}-5-{[(4-methoxybenzyl)(4-dimethylaminobenzyl)amino]carbonyloxyethoxyethoxy}-3-(dimethylamino) pentane,
(k) higher analogs, or
(l) mixtures and combinations thereof.

### R^{a}═(OCH₂)ₙ or CH₃)(CH₂O)ₙ and Z=3-DimethylAmine Class

In certain embodiments, the integrin agonists of Formula (II) comprise one or more integrin agonists, wherein the R^{a} group comprises (OCH₂)ₙ or (OCH₂CH₂)ₙ, wherein n is an integer between 1 and 6 and Z is dimethylamine. In other embodiments, the integrin agonists of Formula (II) comprise one or more of the following integrin activating compounds:
(a) 5-oxo-7-(2-thienyl)-6-(2-thienylmethyl)-2,4-dioxa-6-aza-heptanyl-N,N-dimethylamine,
(b) 5-oxo-7-(3-methoxyphenyl)-6-(3-methoxybenzyl)-2,4-dioxa-6-aza-heptanyl-N,N-dimethylamine,
(c) 5-oxo-7-(3-methoxyphenyl)-6-(4-dimethylaminobenzyl)-2,4-dioxa-6-aza-heptanyl-N,N-dimethylamine,
(d) 5-oxo-7-(4-methoxyphenyl)-6-(4-methoxybenzyl)-2,4-dioxa-6-aza-heptanyl-N,N-dimethylamine,
(e) 5-oxo-7-(4-methoxyphenyl)-6-(4-dimethylaminobenzyl)-2,4-dioxa-6-aza-heptanyl-N,N-dimethylamine,
(f) 7-oxo-9-(2-thienyl)-8-(2-thienylmethyl)-2,4,6-trioxa-8-aza-nonayl-N,N-dimethylamine,
(g) 7-oxo-9-(3-methoxyphenyl)-8-(3-methoxybenzyl)-2,4,6-trioxa-8-aza-undecyl-N,N-dimethylamine,
(h) 7-oxo-9-(3-methoxyphenyl)-8-(4-dimethylaminobenzyl)-2,4,6-trioxa-8-aza-dodecyl-N,N-dimethylamine,
(i) 7-oxo-9-(3-methoxyphenyl)-8-(3-methoxybenzyl)-2,4,6-trioxa-8-aza-dodecyl-(ii) N,N-dimethylamine,
(j) 7-oxo-9-(3-methoxyphenyl)-8-(4-dimethylaminobenzyl)-2,4,6-trioxa-8-aza-dodecyl-N,N-dimethylamine,
(k) 9-oxo-11-(2-thienyl)-10-(2-thienylmethyl)-2,4,6,8-tetraoxa-10-aza-undecyl-N,N-dimethylamine,
(l) 9-oxo-11-(3-methoxyphenyl)-10-(3-methoxybenzyl)-2,4,6,8-tetraoxa-10-aza-pentadecyl-N,N-dimethylamine,
(m) 9-oxo-11-(3-methoxyphenyl)-10-(4-dimethylaminobenzyl)-2,4,6,8-tetraoxa-10-aza-pentadecyl-N,N-dimethylamine,
(n) 9-oxo-11-(4-methoxyphenyl)-10-(4-methoxybenzyl)-2,4,6,8-tetraoxa-10-aza-pentadecyl-N,N-dimethylamine,
(o) 9-oxo-11-(3-methoxyphenyl)-10-(4-dimethylaminobenzyl)-2,4,6,8-tetraoxa-10-aza-pentadecyl-N,N-dimethylamine,
(p) higher analogs, or
(q) mixtures and combinations thereof.

In other embodiments, the integrin activating compounds comprise one or more of the following integrin activating compounds:
(a) 7-oxo-9-(2-thienyl)-8-(2-thienylmethyl)-3,6-dioxa-8-aza-nonanyl-N,N-dimethylamine,
(b) 7-oxo-9-(3-methoxyphenyl)-8-(3-methoxybenzyl)-3,6-dioxa-8-aza-nonanyl-N,N-dimethylamine,
(c) 7-oxo-9-(3-methoxyphenyl)-8-(4-dimethylaminobenzyl)-3,6-dioxa-8-aza-nonanyl-N,N-dimethyl amine,
(d) 7-oxo-9-(4-methoxyphenyl)-8-(4-methoxybenzyl)-3,6-dioxa-8-aza-nonanyl-N,N-dimethylamine,
(e) 7-oxo-9-(4-methoxyphenyl)-8-(4-dimethylaminobenzyl)-3,6-dioxa-8-aza-nonanyl-N,N-dimethyl amine,
(f) 10-oxo-12-(2-thienyl)-11-(2-thienylmethyl)-3,6,9-trioxa-11-aza-dodecyl-N,N-dimethylamine,
(g) 10-oxo-12-(3-methoxyphenyl)-11-(3-methoxybenzyl)-3,6,9-trioxa-11-aza-dodecyl-N,N-dimethyl amine,
(h) 10-oxo-12-(3-methoxyphenyl)-11-(4-dimethylaminobenzyl)-3,6,9-trioxa-11-aza-dodecyl-N,N-dimethylamine,
(i) 10-oxo-12-(3-methoxyphenyl)-11-(3-methoxybenzyl)-3,6,9-trioxa-11-aza-dodecyl-N,N-dimethyl amine,
(j) 10-oxo-12-(3-methoxyphenyl)-11-(4-dimethylaminobenzyl)-3,6,9-trioxa-11-aza-dodecyl-N,N-dimethylamine,
(k) 13-oxo-15-(2-thienyl)-14-(2-thienylmethyl)-3,6,9,12-tetraoxa-14-aza-pentadecyl-N,N-dimethyl amine,
(l) 13-oxo-15-(3-methoxyphenyl)-14-(3-methoxybenzyl)-3,6,9,12-tetraoxa-14-aza-pentadecyl-N,N-dimethylamine,
(m) 13-oxo-15-(3-methoxyphenyl)-14-(4-dimethylaminobenzyl)-3,6,9,12-tetraoxa-14-aza-pentadecyl-N,N-dimethylamine,
(n) 13-oxo-15-(4-methoxyphenyl)-14-(4-methoxybenzyl)-3,6,9,12-tetraoxa-14-aza-pentadecyl-N,N-dimethylamine,
(o) 13-oxo-15-(3-methoxyphenyl)-14-(4-dimethylaminobenzyl)-3,6,9,12-tetraoxa-14-aza-pentadecyl-N,N-dimethylamine,
(p) higher analogs, or
(q) mixtures and combinations thereof.

### Suitable Agents and Materials Surfactants

Zwitterionic Surfactants

Suitable zwitterionic surfactants for use in the present disclosure include, without limitation, phospholipids, betaines, sultaines, sulfobetaine, amidobetaines, imidazoline betaines, or any combination thereof.

Suitable zwitterionic compounds include, without limitation: (1) any compound having the general structure R⁶R⁷R⁸N⁺—R⁹—CO₂⁻, where R⁶, R⁷, and R⁸ are the same or different carbon-containing group, amido carbon-containing group, ether carbon-containing group, or mixtures thereof, and R⁹ is an alkenyl group, alkenyloxide group or mixtures thereof; (2) any compound having the general structure R¹⁰(R⁷R⁸N⁺—R⁹—CO₂⁻)ₙ, where R⁷ and R⁸ are the same or different carbon-containing group, amido carbon-containing group, ether carbon-containing group, or mixtures thereof, R⁹ is an alkenyl group, alkenyloxide group or mixtures thereof, and R¹⁰ is a multivalent substituent having a valency n between 2 and about 6, e.g., CH₂ moiety when n is 2, a CH moiety when n is 3 and a C atom when n is 4; (3) any compound having the general structure R¹²—C(O)—N(R¹¹)—R¹³—N⁺(R⁷R⁸)—R⁹—CO₂⁻, where R⁷, R⁸, R¹¹ and R¹² are the same or different carbon-containing group, amido carbon-containing group, ether carbon-containing group, or mixtures thereof, and R⁹ and R¹³ are the same or different alkenyl group, alkenyloxide group or mixtures thereof; (4) any compound having the general structure R¹⁴—[R¹⁵—C(O)—N(R¹¹)—R¹³—N⁺(R⁷R⁸)—R⁹—CO₂]ₘ, where R⁷, R⁸ and R¹¹ are the same or different carbon-containing group, amido carbon-containing group, ether carbon-containing group, or mixtures thereof, R⁹, R¹³ and R¹⁵ are the same or different alkenyl group, alkenyloxide group or mixtures thereof and R¹⁴ is a multivalent substituent having a valency m between 2 and about 6; other similar ammonium acid zwitterionic agent; or mixtures or combinations thereof. Preferred zwitterionic compounds are betaines such as cocamidopropyl betaine, 5-(1-piperidiniomethyl)-1H-tetrazolide, or similar zwitterionic compounds. Other zwitterionic compounds for use in this invention include, without limitation, phospholipids capable of assuming a zwitterionic state such as phosphatidylcholine, phosphatidylserine, phosphalidylethanolamine, sphingomyelin and other ceramides, as well as various other zwitterionic phospholipids. Preferred sulfo-betaines and related zwitterionic compounds include, without limitation, N-Decyl-N,N-dimethyl-3-ammonio-1-propanesulfonate; Dimethylbenzyl-(3-sulfopropyl)ammonium; Dimethylethyl-(3-sulfopropyl)ammonium; Dimethyl-(2-hydroxyethyl)-(3-sulfopropyl)ammonium; 4-n-Hexylbenzoylamido-propyl-dimethylammoniosulfobetaine; -Methyl-N-(3-sulfopropyl)morpholinium; 4-n-Octylbenzoylamido-propyl-dimethylammoniosulfobetaine; 1-(3-Sulfopropyl)pyridium; N-Tetradecyl-N,N-Dimethyl-3-Ammonio-1-Propanesulfonate, or the like or mixtures or combination thereof.

Exemplary examples of phospholipids include, without limitation, phosphatidylcholine, 1-lysophosphatidylcholine, alkylglycerol monooxygenase, cardiolipin, dinogunellin, 1,2-dioleoyl-sn-glycerophosphoethanolamine, dipalmitoylphosphatidylcholine, dolichyl beta-d-glucosyl phosphate, edelfosine, egg lecithin, soy lecithin, endothelial lipase, glycerophospholipids, hydroxylated lecithins, lecithins, lysophosphatidic acid, lysophosphatidylethanolamine, lysophosphatidylinositol, monolysocardiolipin, myo-inositol trispyrophosphate, perifosine, phosphatidylethanol, phosphatidylethanolamine, phosphatidylglycerol, phosphatidylinositol, phosphatidylinositol (3,4,5)-trisphosphate, phosphatidylinositol 3-phosphate, phosphatidylinositol 3,4-bisphosphate, phosphatidylinositol 3,5-bisphosphate, phosphatidylinositol 4-phosphate, phosphatidylinositol 4,5-bisphosphate, phosphatidylinositol 5-phosphate, phosphatidylinositol phosphate, phosphatidylinositol-4-phosphate 5-kinase, phosphatidylmyo-inositol mannosides, phosphatidylserine, phosphorylcholine, phytosome, PIP2 (disambiguation), POPC, SignaFresh, sphingomyelin, sphingosyl phosphatide, or any mixture thereof.

Exemplary examples of sulfobetaines include, without limitation, Sulfobetaine 8 (SB-8), Sulfobetaine 10 (SB-10), Sulfobetaine 12 (SB-12), Sulfobetaine 14 (SB-14), Sulfobetaine 16 (SB-16) and/or Sulfobetaine 18 (SB-18) available from Taiwan Hopax Chemicals Mfg. Co., Ltd.

Nonionic Surfactants

Suitable nonionic surfactants are categorized by their hydrophilic-lipophilic balance (HLB) number, with a low value (≤10) corresponding to greater lipophilicity and a higher value (≥10) corresponding to higher hydrophilicity. Low HLB (<10) emulsifier include, without limitation, (a) phosphatidylcholine and phosphatidylcholine/solvent mixture such as phosphatidylcholine, phosphatidylcholine in propylene glycol, phosphatidylcholine in medium chain triglycerides, and phosphatidylcholine in safflower oil, (b) unsaturated polyglycolized glycerides such as oleoyl macrogolglycerides and linoleoyl macrogolglycerides, (c) sorbitan esters such as sorbitan monooleate, sorbitan monostearate, sorbitan monolaurate, and sorbitan monopalmitate; or (d) mixtures or combinations thereof. High HLB (>10) emulsifier include, without limitation, (a) polyoxyethylene sorbitan esters such as polysorbate 20, polysorbate 40, polysorbate 60, and polysorbate 80; (b) polyoxyl castor oil derivatives such as Polyoxyl 35 castor oil and Polyoxyl 40 hydrogenated castor oil; (c) polyoxyethylene polyoxypropylene block copolymer such as Poloxamer 188 and Poloxamer 407; (d) saturated polyglycolized glycerides such as lauroyl macrogolglycerides and stearoyl macrogolglycerides; (e) PEG-8 caprylic/capric glycerides such as caprylocaproyl macrogolglycerides; (f) vitamin E derivative such as tocopherol PEG succinate; or (g) mixtures or combinations thereof. Other suitable nonionic surfactants include, without limitation, polyolesters, cyclic polyol esters, polyethylene glycol (PEG) esters, or mixtures and combination thereof. Exemplary examples include, without limitation, sorbitan monofatty and/or polyfatty acid esters, sorbitoal monofatty and/or polyfatty acid esters, mono fatty acid glycerides, polyethylene glycol (PEG) ester surfactants including hydrophilic and/or hydrophobic gelucires such as hydrophilic GELUCIRE® 44/14, lauroyl macrogol glyceride type 1500, hydrophobic GELUCIRE®43/01, GELUCIRE®39/01, GELUCIRE®33/01, or other gelucires; polyglycol modified castor oils such as polyoxyl 35 hydrogenated castor oil, polyoxyl 40 hydrogenated castor oil; polysorbates such as polysorbate 20, 40, 60, 80, etc., and TWEEN® surfactants; and mixtures or combinations thereof.

Sorbitan and Sorbitol Esters

Suitable sorbitan and/or sorbitol esters for use in this disclosure include, without limitation, sorbitan mono ester such as sorbitan caprylate, sorbitan undecylenate, sorbitan laurate, sorbitan palmitate, sorbitan isostearate, sorbitan oleate, sorbitan stearate, etc.; sorbitan sesquiesters such as sesquicaprylate, sorbitan sesquiisostearate, sorbitan sesquioleate, sorbitan sesquistearate, etc.; sorbitan diesters such as sorbitan diisostearate, sorbitan dioleate, sorbitan distearate, etc.; (2002) sorbitan triesters such as sorbitan triisostearate, sorbitan trioleate, sorbitan tristearate, etc.; mixed-chain sorbitan esters such as sorbitan cocoate, sorbitan olivate, sorbitan palmate, sorbitan Theobroma grandiflorum seedate, etc.; or mixtures and combinations thereof. Other sorbitan or sorbitol esters include, without limitation, PEGs sorbitan and sorbitol fatty acid esters including PEG-20 sorbitan cocoate, PEG-40 sorbitan diisostearate, PEG-2 sorbitan isostearate, PEG-5 sorbitan isosteatate, PEG-20 sorbitan isostearate, PEG-40 sorbitan lanolate, PEG-75 sorbitan lanolate, PEG-10 sorbitan laurate, PEG-40 sorbitan laurate, PEG-44 sorbitan laurate, PEG-75 sorbitan laurate, PEG-80 sorbitan laurate, PEG-3 sorbitan oleate, PEG-6 sorbitan oleate, PEG-80 sorbitan palmitate, PEG-40 sorbitan perisostearate, PEG-40 sorbitan peroleate, PEG-3 sorbitan stearate, PEG-6 sorbitan stearate, PEG-40 sorbitan stearate, PEG-60 sorbitan stearate, PEG-30 sorbitan tetraoleate, PEG-40 sorbitan tetraoleate, PEG-60 sorbitan tetraoleate, PEG-60 sorbitan tetrasterate, PEG-160 sorbitan triisostearate; PEG-20 sorbitan triisostearate, Sorbeth-40 hexaoleate, Sorbeth-50 hexaoleate, Sorbeth-30 tetraoleate laurate, Sorbeth-60 tetrastearate, and any mixture thereof. These PEG sorbitans or sorbitols range from tan, waxy solids and amber-colored pastes to clear yellow liquids. Other exemplary nonionic surfactants include, without limitation, polyoxyethylene surfactants such as POE sorbitanmonolaurate (Tween 20, HLB 17), POE sorbitanmonopalmitate (Tween 40, HLB 15.6), POE sorbitanmonostearate (Tween 60, HLB 15.0), POE sorbitanmonooleate (Tween 80, HLB 15.0), POE sorbitantristearate (Tween 65, HLB 10.5), POE sorbitantrioleate (Tween 85, HLB 11.0), POE glycerol trioleate (Tagat TO, HLB 11.5), POE-40-hydrogenated castor oil (solid) Cremophore RH 40, HLB 14.0 to 16.0), POE-35-castor oil (Cremophore EL (liquid), HLB 12.0-14.0), POE (10) oleyl ether (Brij 96, HLB 12.4), POE (23) lauryl ether (Brij 35, HLB 16.9), POE-vitamin E (Alpha-tocopherol TPGS, HLB 13.0), and mixtures or combinations thereof.

Poloxamers

Suitable poloxamers include, without limitation, nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade names Synperonics, Pluronics, and Kolliphor. Because the lengths of the polymer blocks can be customized, many different poloxamers exist that have slightly different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g., P407=poloxamer with a polyoxypropylene molecular mass of 4000 g/mol and a 70% polyoxyethylene content). For the Pluronic and Synperonic tradenames, coding of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobic components; and the last digit x 10 gives the percentage polyoxyethylene content (e.g., L61 indicates a polyoxypropylene molecular mass of 1800 g/mol and a 10% polyoxyethylene content). In the example given, poloxamer 181 (P181)=Pluronic L61 and Synperonic PE/L 61.

Nonionic Neutral Polymers

Suitable nonionic neutral polymers include, without limitation, pH responsive nonionic polymers and temperature sensitive nonionic polymers. Exemplary examples of such pH responsive nonionic polymers include, without limitation, pH responsive dendrimers such as poly-amidoamide (PAMAM), dendrimers, poly(propyleneimine) dendrimers, poly(l-lisine) ester, poly(hydroxyproline), Poly(propyl acrylic acid), poly(methacrylic acid), Carbopol®, Eudragit® S-100, Eudragit® L-100, chitosan, poly(methacrylic acid) (PMMA), PMAA-PEG copolymer, N,N-dimethylaminoethyl methacrylate (DMAEMA), and any mixture thereof. Exemplary examples of temperature sensitive polymer include, without limitation, poloxamers (Pluronics®), prolastin, poly(n-substituted acrylamide), poly(organophosphazene), cyclotriphosphazenes with poly(ethyleneglycol) and amino acid esters, block copolymers of poly(ethylene glycol)/poly(lactic-co-glycolic acid), poly(ethylene glycol) (PEG), poly(propylene glycol) (PPG), PMAA, poly(vinyl alcohol) (PVA), various silk-elastin-like polymers, poly(silamine), poly(vinyl methyl ether) (PVME), poly(vinyl methyl oxazolidone) (PVMO), poly(vinyl pyrrolidone) (PVP), poly(n-vinylcaprolactam), poly(N-vinyl isobutyl amid), poly(vinyl methyl ether), poly(N-vinylcaprolactam) (PVCL), poly(siloxyethylene glycol), poly(dimethylaminoethylmethacrylate), triblock copolymer poly(DL-lactide-co-glycolide-b-ethylene glycol-b-DL-lactide-co-glycolide) (PLGA-PEG-PLGA), cellulose derivatives, alginate, gellan, xyloglucan, and any mixture thereof.

Anionic Surfactants

Suitable anionic surfactants include, without limitation, anionic sulfate surfactant, alkyl ether sulfonates, alkylaryl sulfonates, or mixture or combinations. Preferred sodium or ammonium alcohol ether sulfate surfactants include those having the general formula R¹O—(CH₂CH₂O)ₙSO₃NH₄, where R¹ is a carbon-containing group including an alkyl group, an aryl group, an alkaryl group, an aralkyl group or mixture thereof. Particularly preferred sodium or ammonium alcohol ether sulfate surfactants include short chain sodium or ammonium alcohol ether sulfate surfactants having between 2 and about 10 carbon atoms, especially, between about 4 and 10 carbon atoms and long chain sodium or ammonium alcohol ether sulfate surfactants having between about 10 to about 24 carbon atoms, more particularly, between about 12 and about 18 carbon atoms, especially, between about 12 and about 14 carbon atoms. The sodium ammonium alcohol ether sulfate surfactants are prepared by reacting 1 to 10 moles of ethylene oxide per mole of alkanol, preferred, are prepared by reacting 3 moles of ethylene oxide per mole of alkanol.

Preferred alkylaryl sulfonates including, without limitation, alkyl benzene sulfonic acids and their salts, dialkylbenzene disulfonic acids and their salts, dialkylbenzene sulfonic acids and their salts, alkyltoluene/alkyl xylene sulfonic acids and their salts, alkylnaphthalene sulfonic acids/condensed alkyl naphthalene sulfonic acids and their salts, alkylphenol sulfonic acids/condensed alkylphenol sulfonic acids and their salts, or mixture or combinations thereof.

preferred alkyl ether sulfonates including, without limitation, alkyl ether sulfonates having the general formula R²[—(O—R³O)m-(R⁴O)n-(R⁵)]y where: R²=alkyl, alkenyl, amine, alkylamine, dialkylamine, trialkylamine, aromatic, polyaromatic, cycloalkane, cycloalkene, R³, R⁴═C₂H₄ or C₃H₆ or C₄H₈, R⁴=linear or branched C₇H₁₄SO₃X to C₃₀H₆₀SO₃X when y=1, R⁵=linear or branched C₇H₁₄SO₃X to C₃₀H₆₀SO₃X or H when y>1 but at least one R⁴ must be linear or branched C₇H₁₄SO₃X to C₃₀H₆₀SO₃X, M is greater or equal to 1, n is greater or equal to 0, n+m=1 to 30+, y is greater or equal to 1, X=alkali metal or alkaline earth metal or ammonium or amine.

Cationic Surfactants

Suitable cationic surfactants include, without limitation, Gemini, bis or di quaternary ammonium surfactants such as bis quaternary ammonium halides of bis halogenated ethane, propane, butane, or higher halogenated alkanes, e.g., dichloroethane or dibromoethane, or bis halogenated ethers such as dichloroethylether (DCEE). Preferred bis quaternary ammonium halides are prepared from substituted dimethyl tertiary amines, where the substituent includes between about 4 and about 30 carbon atoms, preferably, between about 6 and about 24 carbon atoms, and particularly, between about 8 and about 24 carbon atoms, and where one or more of the carbon atoms can be replace by an oxygen atom in the form of an ether and/or hydroxyl moiety and/or a nitrogen atom is the form of an amido moiety. Particularly preferred bis quaternary ammonium halides hydrocarbons are prepared from naturally occurring acids, such as fatty acids, synthetic acids, modified naturally occurring acids, or mixture or combinations thereof. Preferred naturally occurring acids are those found in naturally occurring oils such as coconut oil, palm oil, palm kernel oil, soya, safflower oil, sunflower oil, peanut oil, canola oil, or from animals such as tallow oil and its derivatives. Preferred bis quaternary ammonium halides are prepared from disubstituted methyltertiaryamines, where the substituents include between about 4 and about 30 carbon atoms, preferably, between about 6 and about 24 carbon atoms, and particularly, between about 8 and about 24 carbon atoms, and where one or more of the carbon atoms can be replace by an oxygen atom in the form of an ether and/or hydroxyl moiety and/or a nitrogen atom is the form of an amido moiety, such as amidopropyltertiary amines, derived from the reaction of dimethyl aminopropylamine (DMAPA) or similar terminated primary-tertiary diamines, reacted with the above mentioned oils or their corresponding fatty acids, or hydroxy acids. Other preferred cationic surfactants are dimer acids or anhydrides including alkylsubstituted maleic anhydride, alkylsubstituted diethylmalonic acid, or alkylsubstituted higher diacids such as azelaic acid (C9), trimer acids as NTA (nitriloacetic acid), and aconitic acid and trimetellic anhydride are useful though producing a higher trimer. the tertiary amine may be accomplished by reaction of a diamine with a fatty acid or oil, reacting with one amine and then converting the other primary amine to tertiary by the addition of tetrahydrofuran, ethylene oxide, propylene oxide, butylene oxide, epichlorohydrin, or the like and further where the terminal hydrogens of the primary amine can be alkylated using formaldehyde/formic acid mixtures.

### Neutral Lipids

Fatty Acid Esters

Suitable neutral lipids for use in the present disclosure include, without limitation, fatty acid esters, such as alkyl fatty acid esters, and/or polyalkylene oxide fatty acid esters.

Exemplary examples of fatty acid esters comprise esters of any of the fatty acids listed above including, without limitation, mono-alcohol esters, where the mono-alcohol or polyols including 1 carbon atom to 20 carbon atoms, where one or more of the carbon atoms may be replace by O, NR (R is a carbyl or hydrocarbyl group having between 1 and 5 carbon atoms), or S. Exemplary mono-alcohols used to from the free fatty acid esters include methanol, ethanol, propanol, butanol, pentanol or mixtures thereof.

Exemplary examples of fatty acid esters comprise (a) short-chain fatty acid esters (SCFE) include all esters of fatty acids having less than six carbons; (b) medium-chain fatty acid esters (MCFE) include all esters of fatty acids having between 6 to 12 carbons; (c) long-chain fatty acid esters (LCFE) include all esters of fatty acids having 13 to 21 carbons; and (d) very long chain fatty acid esters (VLCFE) include all esters of fatty acids have more than 22 carbons.

Fatty Acid Glycerol Esters

Suitable monoglycerides, diglycerides, and/or triglycerides include monoglycerides, diglycerides, and/or triglycerides derived from animals or plants—animal oils and vegetable oils. Exemplary animal oils include, without limitation, adipose tissue oil, beef tallow, blubber, bone oil, bottlenose oil, butter, butterfat, chicken fat, cod-liver oil, fish oil, ghee, goose grease, halibut-liver oil, hog lard, lanolin, lard, lard oil, lipid, lipoma, margarine, neat s-foot oil, oleo, oleo oil, oleomargarine, porpoise oil, salmon oil, sardine oil, seal oil, shark oil, shortening, sperm oil, suet, tallow, tallow oil, tuna oil, whale oil, wool fat or wool grease, wool oil, or any combination thereof. Exemplary animal oils include, without limitation, coconut oil, corn oil, canola oil, cottonseed oil, olive oil, palm oil, peanut oil, rapeseed oil, safflower oil, sesame oil, soybean oil, sunflower oil, almond oil, beech nut oil, Brazilian nut oil, cashew oil, hazelnut oil, macadamia oil, mongongo nut oil, pecan oil, pine nut oil, pistachio oil, walnut oil, pumpkin seed oil, grapefruit seed oil, lemon oil, orange oil, bitter gourd oil, bottle gourd oil, buffalo gourd oil, butternut squash seed oil, pumpkin seed oil, watermelon seed oil, any mixture thereof.

Exemplary examples of monoglycerides comprise (a) short-chain monoglycerides (SCMs) include all glycerol esters of fatty acids having less than six carbons; (b) medium-chain monoglycerides (MCMs) include all glycerol esters of fatty acids having between 6 to 12 carbons; (c) long-chain fatty acid monoglycerides (LCMs) include all glycerol esters of fatty acids having 13 to 21 carbons; and (d) very long chain fatty acid monoglycerides (VLCMs) include all glycerol esters of fatty acids have more than 22 carbons.

Exemplary examples of diglycerides comprise (a) short-chain diglycerides (SCDs) include all glycerol esters of fatty acids having less than six carbons; (b) medium-chain diglycerides (MCDs) include all glycerol esters of fatty acids having between 6 to 12 carbons; (c) long-chain fatty acid diglycerides (LCDs) include all glycerol esters of fatty acids having 13 to 21 carbons; and (d) very long chain fatty acid diglycerides (VLCDs) include all glycerol esters of fatty acids have more than 22 carbons.

Exemplary examples of triglycerides comprise (a) short-chain triglycerides (SCTs) include all glycerol esters of fatty acids having less than six carbons; (b) medium-chain triglycerides (MCTs) include all glycerol esters of fatty acids having between 6 to 12 carbons; (c) long-chain fatty acid triglycerides (LCTs) include all glycerol esters of fatty acids having 13 to 21 carbons; and (d) very long chain fatty acid triglycerides (VLCTs) include all glycerol esters of fatty acids have more than 22 carbons.

Biocompatible Oils

Suitable biocompatible oils include, without limitation, any oil approved for human, mammal or animal consumption by the FDA or other governmental agency. Exemplary biocompatible oils include, without limitation, plant derived oils or animal derived oils or their derivatives or synthetic oils. In certain embodiments, the natural oils are oils rich in phospholipids such as lecithin oils from soy beans. Exemplary examples of plant derived oils or animal derived oils or their derivatives or synthetic oils include, without limitation, essential oils, vegetable oils an hydrogenated vegetable oils such as peanut oil, canola oil, avocado oil, safflower oil, olive oil, corn oil, soy bean oil, sesame oil, vitamin A, vitamin D, vitamin E, or the like, animal oils, fish oils, krill oil, or the like or mixture thereof.

In certain embodiments, the biocompatible oil is a neutral lipid. Suitable neutral lipids include, without limitation, any neutral lipid such as a triglyceride. For a partial listing of representative neutral lipids, such as the triglycerides, reference is specifically made to U.S. Pat. Nos. 4,950,656 and 5,043,329. Both saturated and unsaturated triglycerides may be employed in the present compositions, and include such triglycerides as tripalmitin (saturated), triolein and trilinolein (unsaturated). However, these particular triglycerides are merely representative examples of useful triglycerides.

Animal fats include, without limitation, lard, duck fat, butter, or mixture or combination thereof.

Vegetable fats include, without limitation, coconut oil, palm oil, cottonseed oil, wheat germ oil, soya oil, olive oil, corn oil, sunflower oil, safflower oil, hemp oil, canola/rapeseed oil, or mixture and combinations thereof.

### Gelatins

Suitable gelatins include chemical materials having an ATC (Anatomical Therapeutic Chemical Classification) registry number B05AA06 and the CAS (Chemical Abstracts Service) registry number 0009000-70-8. Suitable gelatins have the chemical description of purified protein obtained either by partial acid hydrolysis (type A), partial alkaline hydrolysis (type B) or enzymatic hydrolysis of collagen from animals; it may also be a mixture of different types.

These gelatins are described under the following foreign names Gelatina (Latin), Gelatine (German), Gélatine (French), and Gelatina (Spanish).

Gelatins are sold under the following generic names: Gelatin (Official Synonym: Japanese Accepted Name), Gélatine (Official Synonym: Dénomination Commune Française), Gelatina alba (Inofficial Synonym), Gelatina animalis (Inofficial Synonym), UNII-2G86QN327L (Inofficial Synonym), Gelatin (Pharmacopoeia Name: BP 2018, JP XVII, The National Formulary 33, Ph. Eur. 9.3, Ph. Int. 7), Gelatina (Pharmacopoeia Name: Ph. Eur. 9.3, Ph. Int. 7), Gelatine (Pharmacopoeia Name: Ph. Eur. 9.3), Gélatine (Pharmacopoeia Name: Ph. Eur. 9), Purified Gelatin (Pharmacopoeia Name: JP XVII), Absorbable Gelatin (Pharmacopoeia Name: USP 41), Gelatine Polysuccinat zur parenteralen Anwendung (Inofficial Synonym), Gelofusin (Inofficial Synonym), Modified fluid gelatin (Inofficial Synonym), and Modifizierte Gelatine (Inofficial Synonym).

Gelatins are sold under the following brand names: Colagenan manufactured by Herbarium Laboratdrio Botânico, Brazil, Gelatina Pharma Arte manufactured by Pharma Arte, Paraguay, Gelfilm manufactured by Pfizer, New Zealand, Gelfoam manufactured by Pfizer, New Zealand, Gelofusine manufactured by B. Braun, France, Gelofusine manufactured by [+Sodium Chloride] B. Braun, Hungary; B. Braun Medical, Latvia; JV Healthcare Limited, Malta, Geloplasma manufactured by Fresenius Kabi, Estonia; Fresenius Kabi, Romania; Fresenius Kabi Austria, Austria; Fresenius-Kabi, Argentina, Geloplasma [+Magnesium Chloride, +Potassium Chloride, +Sodium Chloride, +Sodium Lactate] manufactured by Fresenius Kabi, Latvia, Glycerin AD [+Glycerol] manufactured by Pharco, Egypt, Glycerin Inf [+Glycerol] manufactured by Pharco, Egypt, Glycerin Pharco [+Glycerol] manufactured by Pharco, Kuwait, Glycerol Adult [+Glycerol] manufactured by Sedico, Egypt, Glycerol Infant [+Glycerol] manufactured by Sedico, Egypt, Intragel [+Sodium Chloride] [veterinary use] manufactured by Zoetis, United States, Stomahesive [+Pectin, +Carmellose] manufactured by Bristol-Myers Squibb, Uruguay, Tego manufactured by Armstrong Laboratorios de Mexico, Mexico, Valcatil [+Cysteine, +Methionine] manufactured by Panalab, Argentina; Panalab, Paraguay, Gelfilm manufactured by Pfizer Japan, Japan, Gelfoam manufactured by Pfizer, Singapore; Pfizer Japan, Japan, Spongel manufactured by Astellas, Japan, Eufusin manufactured by Medacta Italia, Italy, Galafundin [+Calcium Chloride, +Sodium Chloride] manufactured by Braun, Kuwait, Gelafundin manufactured by Buminusantara Bestari Perkasa/B Braun, Indonesia; Pisa, Mexico, Gelafundin 4% manufactured by B. Braun, Germany, Gelafundin ISO manufactured by B. Braun, Germany, Gelafundina manufactured by B. Braun Medical, Spain, Gelafusal manufactured by Biomed, New Zealand; Serumwerk Bernburg, Germany, Gelaspan manufactured by B. Braun Medical, Spain; B. Braun B. Braun Melsungen, Romania; Braun, Norway, Gelaspan 4% manufactured by B. Braun Medical, Estonia, Gelofusine manufactured by B. Braun, Italy; B. Braun Medical, Denmark; B. Braun Medical, Ecuador; B. Braun Medical, Netherlands; B. Braun Melsungen, Serbia; B. Braun B. Braun Melsungen, Romania; Biomed, New Zealand, Gelofusine [+Sodium Chloride] manufactured by B. Braun, Bulgaria; B. Braun, Czech Republic; B. Braun Austria, Austria; B. Braun Medical, Hong Kong; Braun, Sweden, Geloplasma manufactured by Fresenius Kabi, Spain, Geloplasma [+Magnesium Chloride, +Potassium Chloride, +Sodium Chloride, +Sodium Lactate] manufactured by Fresenius Kabi, Czech Republic; Fresenius Kabi France, Slovenia, Infuplas manufactured by Fresenius Kabi, Italy, Isoplex 4% manufactured by Beacon Pharmaceuticals, United Kingdom, Volplex 4% manufactured by Beacon, and Malta; Beacon Pharmaceuticals, United Kingdom.

### Excipients or Adjuvants

The formulation or compositions of this disclosure can also include other chemicals, such as anti-oxidants (e.g., Vitamin A, C, D, E, etc.), trace metals and/or polyvalent cations (aluminum, gold, copper, zinc, calcium, etc.), surface-active agents and/or solvents (e.g., propylene glycol/PPG, dimethylsulfoxide/DMSO, medium chain triglycerides/MCT, etc.), non-toxic dyes and flavor enhancers may be added to the formulation as they are being prepared to improve stability, fluidity/spreadability, permeability, effectiveness and consumer acceptance. These additives, excipients, and/or adjuvants may also function as active agents.

Suitable organic solvents for use in the present disclosure, without limitation, ethanol, n-propanol, n-butanol, n-pentanol, n-hexanol, or any combination thereof.

### EXPERIMENTS OF THE INVENTION CAPSULE FILL FORMULATION

### Example 1

Preferred Capsule Fill Formulation

The preferred capsule solution comprising suitable for soft gelatin or two-piece capsule encapsulation for 7HP349, 50 mg dosage form is presented in Table 5. The capsule-fill formulation contains 5% w/w 7HP349+14% w/w ethanol+81% w/w Phosal 53 MCT, a soy phosphatidylcholine (PC)-containing oil, the same excipients as present in the 7HP349 Capsule drug product. All materials used in formulation development and intended for use in the Placebo are GMP, qualified, compendial inactive ingredients.

TABLE 5
7HP349 Dosage Form Unit Composition, Pharmaceutical
Function, and Quality Standards

| | | Strength | | Pharmaceutical | Quality |
|---|---|---|---|---|---|
| Component | | mg per capsule | % w/w | Function | Standard |
| API | 7HP349 | 50.0 | 3.3% | Active | In-house |
| Excipients | Phosal 53 MCT | 850.0 | 55.8% | Carrier | In-house |
| | anhydrous Ethanol | 147.0 | 9.6% | Co-solvent | USP/NF |
| | Gelatin, Glycerin | 477.6 | 31.3% | Capsule shell, | USP/NF |
| | FD&C Yellow #6 | | | Plasticizer, Color | |

| | | |
|---|---|---|
| Total Capsule Weight | 1524.6 | 100% |

The components in Phosal all comply with USP/NF and Ph. Eur. The ethanol content in the bulk fill formulation is 14.7%. A minimum of 10% ethanol is adequate to maintain API solution stability. The excess ethanol added in the bulk fill formulation is to accommodate loss during capsule drying.

Phosal 53 MCT soy lecithin-containing oil has been shown to improve the oral bioavailability of hydrophobic small molecules. The ingredients and compendial status of the ingredient are summarized in Table 6.

TABLE 6
Phosal 53 MCT Composition and Compendial Status of Ingredients

| | | Maximum Oral | Per Capsule | |
|---|---|---|---|---|
| | | Potency per FDA | Derived from | |
| | | Inactive Ingredient | Phosal 53 | |
| Ingredients | w/w % | Guide | MCT (mg) | Monograph |
| Phosphatidylcholine | >50% | No limit as food, 325 | 492.5Embodiment 43 | USP/FCC |
| | | mg oral capsule | through Embodiment | |
| | | | 45 | |
| Caprylic/Capric | 25-50% | 3390 mg/5 mL | 405 | Ph. Eur. |
| Triglyceride | | | | |
| Alcohol | 1-5% | 356.28 mg/1 mL | 40.5Embodiment 43 | Ph. Eur. |
| | | | through Embodiment | |
| | | | 45 | |
| Sunflower Seed Oil | 1-5% | Not available | 40.5Embodiment 43 | FCC |
| Glyceride | | | through Embodiment | |
| | | | 45 | |
| Oleic acid | 1-5% | 617.7 mg | 40.5Embodiment 43 | Ph. Eur. |
| | | | through Embodiment | |
| | | | 45 | |
| Ascorbyl Palmitate | 0.1-1% | 12.0 mg | 8.1Embodiment 43 | Ph. Eur |
| | | | through Embodiment | |
| | | | 45 | |
| α-tocopherol | 0.1-1% | 42.5 mg | 8.1 | Ph. Eur. |

The calculations are based on maximum ranges reported by manufacturer in 50 mg capsule

### Formulation Development

### Example 2

Physical Stability of 7HP349 Fill Formulation

The physical stability of different 7HP349 fill formulations containing Phosal 53 MCT were tested at Eurofins Advinus Limited at 2 to 8° C. (long-term), 25±2° C. @ 60±5% RH (ambient) and 40±2° C. @ 75±5% RH (accelerated) storage conditions for 3 months. The test conditions for physical stability are tabulated in Table 7. The compositions of the different 7HP349 formulations were tested as shown in Table 8.

TABLE 7
Test Conditions for Evaluation of Physical
Stability of 7HP349 Fill Formulation

| | | Quantity/ | Frequency of |
|---|---|---|---|
| Study | Storage Conditions | Units | Testing (Months) |
| Long-term | 2 ± 8° C. | 12 samples | 1, 2 and 3 |
| Ambient | 25 ± 2° C. at | 12 samples | 1, 2 and 3 |
| | 60 ± 5% RH | | |
| Accelerated | 40 ± 2° C. at | 12 samples | 1, 2 and 3 |
| | 75 ± 5% RH | | |

TABLE 8
Different 7HP349 Formulations Tested for Physical Stability

% Composition

| ID | 7HP349 | Ethanol | Phosal 53 MCT | Corn Oil |
|---|---|---|---|---|
| Form-1 | 5 | 10.6 | 84.4 | 0.0 |
| Form-2 | | 11.7 | 64.0 | 19.3 |
| Form-3 | | 12.8 | 42.5 | 39.7 |
| Form-4 | | 13.9 | 21.0 | 60.1 |
| Form-5 | 7 | 10.7 | 82.3 | 0.0 |
| Form-6 | | 11.8 | 61.2 | 20.0 |
| Form-7 | | 12.8 | 42.2 | 38.0 |
| Form-8 | | 13.9 | 21.1 | 58.0 |
| Form-9 | 8 | 10.8 | 81.2 | 0.0 |
| Form-10 | | 11.8 | 61.0 | 19.2 |
| Form-11 | | 12.9 | 41.0 | 38.1 |
| Form-12 | | 14 | 20.0 | 58.0 |

The ethanol content in Phosal 53 MCT is 5.2%. The total ethanol content is the sum of the ethanol content of Phosal 53 MCT and added ethanol, so that the resulting content is ˜15%.

Physical Stability Summary

All the formulation samples were tested for precipitation, color change, and phase separation during the storage period. To summarize, all formulation samples stored under ambient conditions (25±2° C. at 60±5% RH) were found to be stable for 3 months without any change in physical appearance. Some formulation samples stored under refrigerated (2 to 8° C.) and accelerated conditions (40±2° C. @ 75±5% RH) showed phase separation with globules and precipitate, which turned into a clear solution after vortex mixing and sonication.

TABLE 9
Physical Stability Summary of 7HP349 Formulations at 3 Months

| | | | Storage Conditions | | |
|---|---|---|---|---|---|
| | % Composition | | | Ambient | Accelerated |
| | | Phosal 53 | Long-term | (25 ± 2° C. @ | (40 ± 2° C. @ |
| ID | 7HP349 | MCT | (2 ± 8° C.) | 60 ± 5% RH) | 75 ± 5% RH) |
| Form-1 | 5 | 84.4 | Single-phase clear | Single-phase | Single-phase |
| | | | solution | clear solution | clear solution |
| Form-2 | | 64.0 | Single-phase clear | Single-phase | Single-phase |
| | | | solution | clear solution | clear solution |
| Form-3 | | 42.5 | Single-phase clear | Single-phase | Single-phase |
| | | | solution | clear solution | clear solution |
| Form-4 | | 21.0 | Single-phase turbid | Single-phase | Single-phase |
| | | | solution; clear | clear solution | clear solution |
| | | | solution after | | |
| | | | sonication | | |
| Form-5 | 7 | 82.3 | Phase separation | Single-phase | Single-phase |
| | | | with small | clear solution | clear solution |
| | | | globules; single- | | |
| | | | phase clear solution | | |
| | | | after sonication | | |
| Form-6 | | 61.2 | Phase separation | Single-phase | Single-phase |
| | | | with small | clear solution | clear solution |
| | | | globules; single- | | |
| | | | phase clear solution | | |
| | | | after sonication | | |
| Form-7 | | 42.2 | Phase separation | Single-phase | Single-phase |
| | | | with small | clear solution | clear solution |
| | | | globules; single- | | |
| | | | phase clear solution | | |
| | | | after sonication | | |
| Form-8 | | 21.1 | Phase separation- | Single-phase | Single-phase |
| | | | like precipitate with | clear solution | clear solution |
| | | | turbid solution; | | |
| | | | single-phase clear | | |
| | | | solution after | | |
| | | | sonication | | |
| Form-9 | 8 | 81.2 | Phase separation | Single-phase | Phase |
| | | | with large globules; | clear solution | separation-like |
| | | | single-phase clear | | precipitate with |
| | | | solution after | | turbid solution; |
| | | | sonication | | single-phase |
| | | | | | clear solution |
| | | | | | after sonication |
| Form-10 | | 61.0 | Phase separation | Single-phase | Turbid solution; |
| | | | with large globules; | clear solution | clear solution |
| | | | single-phase clear | | after sonication |
| | | | solution after | | |
| | | | sonication | | |

### Example 3

Dose Form Characterization

A summary of the initial assay values and impurity levels in the different fill formulations and different fill-shell (two-piece capsule) combinations tested are shown in Table 10. The initial assay values and impurity levels in two development batches of 7HP349 Capsules, 50 mg are tabulated in Table 11 and Table 12. In all formulations, 7HP349 remained stable and no new impurities were identified.

TABLE 10
Initial Purity and Related Substances of Different 7HP349
Fill Formulations and Fill-Shell Combinations Evaluated

| | | | | Initial Values (%) | |
|---|---|---|---|---|---|
| | | | | | Total |
| | Composition (% w/w) | | | Chromatographie | Related |
| | | | Phosal | Purity of | Substance |
| ID/Sample | 7HP349 | Ethanol | 53 MCT | 7HP349 | Impurities |
| U28 | 5 | 10 | 85 | 99.47 | 0.41 |
| U38 | 5 | 0 | 95 | 99.5 | 0.38 |
| U39 | 5 | 3 | 92 | 99.47 | 0.44 |
| Capsule Fill Alone | 5 | 5 | 90 | 99.1 | 0.74 |
| | 5 | 7.5 | 87.5 | 103.1 | 0.76 |
| | 5 | 10 | 85 | 102.2 | 0.74 |
| HMPC Vcaps | 5 | 5 | 90 | 101.1 | 0.73 |
| | 5 | 7.5 | 87.5 | 101.6 | 0.74 |
| | 5 | 10 | 85 | 100.3 | 0.74 |
| HPMC Vcaps Plus | 5 | 5 | 90 | 99.7 | 0.74 |
| | 5 | 7.5 | 87.5 | 102.7 | 0.78 |
| | 5 | 10 | 85 | 98.5 | 0.76 |
| HPMC QualiV | 5 | 5 | 90 | 101.7 | 0.75 |
| | 5 | 7.5 | 87.5 | 101.9 | 0.74 |
| | 5 | 10 | 85 | 100.5 | 0.74 |
| Gelatin Coni-Snap | 5 | 5 | 90 | 99.1 | 0.74 |
| (Hard) | 5 | 7.5 | 87.5 | 97 | 0.78 |
| | 5 | 10 | 85 | 97.1 | 0.73 |
| Gelatin ACG (Soft) | 5 | 5 | 90 | 99.1 | 0.63 |

Phosal 53 MCT contains ˜5% ethanol. The ethanol concentrations mentioned here are further additions. Absolute % values for chromatographic purity assay for fill formulations U28, U38 and U39. % label claim for shell fill and capsules.

### Example 4

The following example illustrates a composition that may be administered parenterally, e.g., intravenously, intra-arterially, intramuscularly, or by injection, to a patient, wherein the composition has anti-solid tumor activity, improves antigen presentation before, during, and/or after antigen administration, enhances antigen presentation cells (APCs) activity, enhances an immune response to vaccines before, during, and/or after vaccine administration, enhances T-cells activity, natural or antigen activated T-cells, enhances therapeutic antibody activity before, during, and/or after therapeutic antibody administration, enhances checkpoint inhibitor activity therapeutic antibody checkpoint inhibitor administration, and/or enhances hematopoietic stem cell bone marrow reconstitution before, during, and/or after hematopoietic stem cell administration.

The composition comprises the following ingredient ranges:

| Ingredient | wt. % | wt. % |
|---|---|---|
| 7HP349 | 1.5 | 4.2 |
| Phosal 53 MCT | 60.6 | 50.0 |
| Ethanol | 7.6 | 12.5 |
| Gelatin (Type 195) | 22.7 | 20.8 |
| Sorbitol-glycerin blend | 7.6 | 12.5 |
| Water | 0.0 | 0.0 |
| Total | 100.0 | 100.0 |

Phosal 53 MCT comprises the following range of ingredients:

| Ingredient | wt. % | wt. % |
|---|---|---|
| Phosphatidylcholine | 50.0 | 55.0 |
| Caprylic/Capric Triglyceride | 44.8 | 27.0 |
| Alcohol | 3.0 | 6.0 |
| Sunflower Seed Oil Glyceride | 1.0 | 5.0 |
| Oleic acid | 1.0 | 5.0 |
| Ascorbyl Palmitate | 0.1 | 1.0 |
| a-tocopherol | 0.1 | 1.0 |
| Total | 100.0 | 100.0 |

In certain embodiments, the composition comprises the following ingredient ranges:

| Ingredient | wt. % | wt. % |
|---|---|---|
| 7HP349 | 1.50 | 4.20 |
| ZISs | 30.30 | 27.50 |
| NLs | 28.48 | 19.50 |
| Organic Solvents | 9.42 | 15.50 |
| Gelatins | 22.70 | 20.80 |
| Sorbitol-glycerin blends | 7.60 | 12.50 |
| Total | 100.00 | 100.00 |

In certain embodiments, the ZISs comprise about 90 wt. % phosphatidylcholine and about 10 wt. % lysophosphatidylcholine, the organic solvent comprises an alcohol, and the NLs comprise about comprise between about 94.12 wt. % triglycerides and about 99.56 wt. % of triglycerides, between about 2.94 wt. % and about 0.22 wt. % of an ascorbyl fatty acid ester, and between about 2.94 wt. % and about 0.22 wt. % of α-tocopherol. In other embodiments, the organic solvent comprises ethanol.

in certain embodiments, the carrier comprises between about 30.76 wt. % and about 28.71 wt. % of one or more ZISs, between about 28.91 wt. % and about 20.35 wt. % of one or more NLs, between about 9.56 wt. % and about 16.18 wt. % of one or more organic solvents, between about 23.05 wt. % and about 21.71 wt. % of one or more gelatins, and between about 7.72 wt. % and about 13.05 wt. % of a bend of sorbitol and glycerin.

The composition forms a stable emulsion when added to an aqueous injectable pharmaceutically acceptable carrier.

### Pharmacokinetic Examples

### Example 5

Several targeted 7HP349 solutions suitable for use in liquid fill capsules were tested. Non-lipid-based solutions with high surfactant levels, e.g., “Solution” provided limited bioavailability. Medium chain triglycerides and high surfactants, provided greater absorption (Formulation Q), while addition of phospholipids to MCT in U28 further improve bioavailability.

TABLE 11
Formulation U28 vs. Other Formulations

| | 7HP349 | | | | | |
|---|---|---|---|---|---|---|
| | Dose | | | | | |
| | (mg/kg, | | Cₘₐₓ | AUCₗₐₛₜ | | |
| Species | PO) | Formulation | ng/mL | ng · h/mL | % F | Remarks |
| Rat | 60 | Formulation Q | 176 | 933 | 1.7 | |
| | 60 | Formulation Q | 85.7 | 617 | 1.1 | |
| | 60 | Formulation Q | 59.5 | 311 | 0.6 | |
| | 15 | Formulation U28 | 189 | 3130 | 22.4 | |
| Dog | 40 | Solution Formulation | 54.6 | 145 | 0.4 | |
| | 40 | Formulation Q | 199 | 608 | 1.9 | |
| | 40 | Formulation U28 | 426 | 1805 | 5.6 | |
| | 40 | Formulation U28 | 708 | 4290 | 13.3 | Combined administration |
| | | | | | | with Ketoconazole, |
| | | | | | | which is a CYP3A4 |
| | | | | | | inhibitor. Increased % F |
| | | | | | | suggests the importance |
| | | | | | | of long chain fatty acids |
| | | | | | | in TG or PL to improve |
| | | | | | | lymphatic partitioning to |
| | | | | | | avoid first pass loss. |
| Monkey | 70 | Formulation Q | 51 | 1280 | 2.8 | Immediate release |
| | | | | | | capsule, fasted |
| | 70 | Formulation Q | 53.7 | 1460 | 3.2 | Enteric coated capsule, |
| | | | | | | fasted |
| | 70 | Formulation Q | 106 | 1440 | 3.2 | Immediate release |
| | | | | | | capsule, high fat fed |

Formulation Q: 7HP349 − 1.0 g, Capmul MCM − 2.125 g, Polysorbate 80 − 1.875 g.
Formulation U28: 7HP349 − 5% w/w, Ethanol − 10% w/w, Phosal 53 MCT − 85% w/w.
Solution Formulation: 5% DMSO + 10% Cremophor EL + 35% PEG400 + 0.1M citrate buffer pH 2.7

Preferred lipid-based formulation based on U28 provided adequate exposure in human. The optimal PK exposure to engage integrins and improve adjuvant activity is systemic plasma exposure of AUC0-t between about 50 ng/mL/hr and about 100 ng/mL/hr.

TABLE 12
Human SAD PK Data

| | 7HP349 | Tₘₐₓ (h) | | Cₘₐₓ | T_{1/2} | AUCₗₐₛₜ | AUC_{0-10 h} |
|---|---|---|---|---|---|---|---|
| Cohort | Dose (mg) | (Range) | | (ng/mL) | (h) | (ng · h/mL) | (ng · h/mL) |
| 1 | 50 | 2.5 | (1-6) | 10.2 ± 8.17 | 8.9 | 31.8 ± 21.5 | 12.5 |
| 2 | 100 | 2 | (0.5-2) | 36.1 ± 16.4 | 20.6 ± 15.0 | 165 ± 113.3 | 115 ± 57.5 |
| 3 | 200 | 2 | (2) | 165.6 ± 61.8 | 34.6 ± 20.7 | 755 ± 241 | 571 ± 195 |
| 4 | 300 | 2 | (1-6) | 321 ± 121.8 | 29.7 ± 15.4 | 1350 ± 316 | 938 ± 282 |

Data represents Mean ± SD (n = 6 per cohort), except for Tₘₐₓ, which is represented as Median (Range)

### EMBODIMENTS OF THE DISCLOSURE

Embodiment 1. A carrier composition comprising:
one or more surfactants (SRFs), and
one or more neural lipids (NLs).

Embodiment 2. A pharmaceutical composition comprising:
a carrier composition comprising:
   one or more SRFs, and
   one or more NLs, and
one or more poorly water-soluble pharmaceutical agents.

Embodiment 3. A pharmaceutical composition comprising:
a soft gel capsule filled with a fill composition comprising:
   a carrier composition comprising:
      one or more SRFs, and
      one or more NLs, and
   one or more poorly water-soluble pharmaceutical agents.

Embodiment 4. The Embodiments of 1, 2, or 3, further comprising:
one or more organic solvents.

Embodiment 5. The Embodiments 1, 2, 3, or 4, wherein the one or more SRFs comprise:
one or more zwitterionic surfactants (ZISs), or
one or more nonionic surfactants (NISs), or
any mixture or combination thereof.

Embodiment 6. The Embodiment 5, wherein the one or more SRFs further comprise:
one or more ionic surfactants (ISs).

Embodiment 7. The Embodiments of 1, 2, 3, or 4, wherein the one or more SRFs comprise:
one or more ZISs.

Embodiment 8. The Embodiments of 1, 2, 3, or 4, wherein the one or more SRFs comprise:
one or more NISs.

Embodiment 9. The Embodiments of 1, 2, 3, or 4, wherein the one or more SRFs comprise:
one or more ZISs and
one or more NISs.

Embodiment 10. The Embodiment of 6, wherein the one or more SRFs comprise:
one or more ZISs,
one or more NISs, and
one or more ISs.

Embodiment 11. Any of the preceding Embodiments, wherein:
the one or more SRFs are present in an amount between about 5 wt. % and about 95 wt. % or any subrange thereof, and
the one or more NLs are present in an amount between about 95 wt. % and about 5 wt. % or any subrange thereof.

Embodiment 12. Any of the preceding Embodiments, wherein:
between about 99 wt. % and about 75 wt. % of an SRF/NL mixture comprising the one or more SRFs and the one or more NLs thereof or any subrange thereof, and
between about 1 wt. % and about 25 wt. % the one or more organic solvents or any subrange thereof.

Embodiment 13. Any of the preceding Embodiments, wherein the one or more SRFs comprise:
between about 99 wt. % to about 90 wt. % of a SRF mixture comprising one or more ZISs, one or more NISs, or any mixture thereof or any subrange thereof, and
between about 1 wt. % and about 10 wt. % of the one or more ISs or any subrange thereof.

Embodiment 14. Any of the preceding Embodiments, wherein:
the carrier composition is present in an amount between about 75 wt. % and about 97 wt. % or any subrange thereof, and
the one or more poorly water-soluble pharmaceutical agents are present in an amount between about 25 wt. % and about 1 wt. % or any subrange thereof.

Embodiment 15. Any of the preceding Embodiments, wherein the one or more NLs comprise:
between about 0 wt. % and about 20 wt. % of a SC mixture comprising one or more SCMs, one or more SCDs, one or more SCTs, one or more SCFEs, or any mixture thereof or any subrange thereof, and
between about 100 wt. % and about 80 wt. % of a NL mixture comprising one or more MCMs, one or more MCDs, one or more MCTs, one or more MCFEs, one or more LCMs, one or more LCDs, one or more LCTs, one or more LCFEs, one or more VLCMs, one or more VLCDs, one or more VLCTs, one or more VLCFEs, or any mixture thereof or any subrange thereof.

Embodiment 16. Any of the preceding Embodiments, wherein the one or more NLs comprise:
between about 0 wt. % and about 20 wt. % of a SC mixture comprising one or more SCMs, one or more SCDs, one or more SCTs, one or more SCFEs, or any mixture thereof or any subrange thereof, and
between about 100 wt. % and about 80 wt. % of a MC mixture comprising one or more MCMs, one or more MCDs, one or more MCTs, one or more MCFEs, or any mixture thereof or any subrange thereof.

Embodiment 17. Any of the preceding Embodiments, wherein the one or more NLs comprise:
between about 0 wt. % and about 20 wt. % of a SC mixture comprising one or more SCMs, one or more SCDs, one or more SCTs, one or more SCFEs, or any mixture thereof or any subrange thereof, and
between about 100 wt. % and about 80 wt. % of a LC mixture comprising one or more LCMs, one or more LCDs, one or more LCTs, one or more LCFEs, or any mixture thereof or any subrange thereof.

Embodiment 18. Any of the preceding Embodiments, wherein the one or more NLs comprise:
between about 0 wt. % and about 20 wt. % of a SC mixture comprising one or more SCMs, one or more SCDs, one or more SCTs, one or more SCFEs, or any mixture thereof or any subrange thereof, and
between about 100 wt. % and about 80 wt. % of a VLC mixture comprising one or more VLCMs, one or more VLCDs, one or more VLCTs, one or more VLCFEs, or any mixture thereof or any subrange thereof.

Embodiment 19. Any of the preceding Embodiments, wherein the one or more NLs comprise:
a MC mixture comprising one or more MCMs, one or more MCDs, one or more MCTs, one or more MCFEs, or any mixture thereof or any subrange thereof.

Embodiment 20. Any of the preceding Embodiments, wherein the one or more NLs comprise:
an LC mixture comprising one or more LCMs, one or more LCDs, one or more LCTs, one or more LCFEs, or any mixture thereof or any subrange thereof.

Embodiment 21. Any of the preceding Embodiments, wherein the one or more NLs comprise:
a VLC mixture comprising one or more VLCMs, one or more VLCDs, one or more VLCTs, one or more VLCFEs, or any mixture thereof or any subrange thereof.

Embodiment 22. Any of the preceding Embodiments, wherein the one or more NLs comprise:
one or more MCMs, one or more MCDs, one or more MCTs, one or more MCFEs, or any mixture thereof or any subrange thereof, and
one or more LCMs, one or more LCDs, one or more LCTs, one or more LCFEs, or any mixture thereof or any subrange thereof.

Embodiment 23. Any of the preceding Embodiments, wherein the one or more NLs comprise:
one or more MCMs, one or more MCDs, one or more MCTs, one or more MCFEs, or any mixture thereof or any subrange thereof, and
one or more LCMs, one or more LCDs, one or more LCTs, one or more LCFEs, or any mixture thereof or any subrange thereof.

Embodiment 24. Any of the preceding Embodiments, wherein the one or more NLs comprise:
one or more MCMs, one or more MCDs, one or more MCTs, one or more MCFEs, or any mixture thereof or any subrange thereof,
one or more LCMs, one or more LCDs, one or more LCTs, one or more LCFEs, or any mixture thereof or any subrange thereof, and
one or more VLCMs, one or more VLCDs, one or more VLCTs, one or more VLCFEs, or any mixture thereof or any subrange thereof.

Embodiment 25. Any of the preceding Embodiments, wherein the one or more NLs comprise:
one or more LCMs, one or more LCDs, one or more LCTs, one or more LCFEs, or any mixture thereof or any subrange thereof, and
one or more VLCMs, one or more VLCDs, one or more VLCTs, one or more VLCFEs, or any mixture thereof or any subrange thereof.

Embodiment 26. Any of the preceding Embodiments, wherein the one or more poorly water-soluble pharmaceutical agents comprising:
one or more poorly water-soluble, small molecule integrin agonist adjuvant pharmaceutical agents.

Embodiment 27. The Embodiment 26, wherein the one or more poorly water-soluble pharmaceutical agents have Log P values in n-octanol v. water of greater than 5 (Log P>5).

Embodiment 28. A pharmaceutical carrier composition comprising:
a carrier comprising:
   between about 30.76 wt. % and 28.71 wt. % of one or more ZISs,
   between about 28.91 wt. % and 20.35 wt. % of one or more NLs,
   between about 9.56 wt. % to 16.18 wt. % of one or more organic solvents,
   between about 23.05 wt. % to 21.71 wt. % of one or more gelatins, and
   between about 7.72 wt. % to 13.05 wt. % of a blend of sorbitol and glycerin.

Embodiment 29. A pharmaceutical composition comprising:
between about 1.50 wt. % and about 4.20 wt. % of one or more one or more poorly water-soluble, small molecule integrin agonist adjuvant pharmaceutical agents,
a carrier comprising:
   between about 30.30 wt. % and about 27.50 wt. % of one of more ZISs,
   between about 28.48 wt. % and about 19.50 wt. % of one or more NLs,
   between about 9.42 wt. % and about 15.50 wt. % of one or more organic solvents,
   between about 22.70 wt. % and about 20.80 wt. % of one or more gelatins,
   between about 7.60 wt. % and about 12.50 wt. 5 of a blend of sorbitol, and
   glycerin, and
   essentially free of water,
a pharmaceutical acceptable aqueous injectable system,
wherein the composition comprises a stable injectable emulsion.

Embodiment 30. An injectable pharmaceutical composition comprising:
a pharmaceutical system comprising:
   between about 1.50 wt. % and about 4.20 wt. % of one or more one or more poorly water-soluble, small molecule integrin agonist adjuvant pharmaceutical agents,
   a carrier comprising:
      between about 30.30 wt. % and about 27.50 wt. % of one of more ZISs,
      between about 28.48 wt. % and about 19.50 wt. % of one or more NLs,
      between about 9.42 wt. % and about 15.50 wt. % of one or more organic solvents,
      between about 22.70 wt. % and about 20.80 wt. % of one or more gelatins,
      between about 7.60 wt. % and about 12.50 wt. 5 of a blend of sorbitol, and glycerin, and
      essentially free of water, and
a pharmaceutical acceptable aqueous injectable system,
wherein the composition comprises a stable injectable emulsion.

Embodiment 31. The Embodiments of 28, 29, or 30, wherein a weight ratio of the pharmaceutical system and the pharmaceutical acceptable aqueous injectable system is between about 1:2 and 1:10.

Embodiment 32. The Embodiments of 28, 29, 30 or 31, wherein the lecithin comprises:
one or more ZISs, and
one or more NLs.

Embodiment 33. Any of the previous Embodiments, wherein:
the one or more SRFs comprising:
   one or more zwitterionic surfactants (ZISs),
   one or more nonionic surfactants (NISs), or
   any combination thereof;
the one or more NLs comprising:
   one or more short chain monoglycerides (SCMs),
   one or more middle chain monoglycerides (MCMs),
   one or more long chain monoglycerides (LCMs),
   one or more long chain monoglycerides (VLCMs),
   one or more short chain diglycerides (SCDs),
   one or more middle chain diglycerides (MCDs),
   one or more long chain diglycerides (LCDs),
   one or more long chain monoglycerides (VLCDs),
   one or more short chain triglycerides (SCTs),
   one or more middle chain triglycerides (MCTs),
   one or more long chain triglycerides (LCTs),
   one or more long chain monoglycerides (VLCTs),
   one or more short-chain fatty acid esters (SCFE),
   one or more medium-chain fatty acid esters (MCFE),
   one or more long-chain fatty acid esters (LCFE),
   one or more very long chain fatty acid esters (VLCFE), or
   any mixture thereof, or
the one or more organic solvents comprising:
   one or more alcohols,
   one or more non-alcohol organic solvent, or
   any mixture thereof.

Embodiment 34. Any of the preceding Embodiments, wherein the one or more poorly water-soluble pharmaceutical agents comprising:
one or more poorly water-soluble, small molecule integrin agonist adjuvant pharmaceutical agents.

Embodiment 35. The Embodiment of 34, wherein the one or more poorly water-soluble, small molecule integrin agonist adjuvant pharmaceutical agents comprising:
one or more compounds of Formula (I),
one or more compounds of Formula (II), or
any mixture thereof of two or more compounds of Formulas (I) and (II).

Embodiment 36. A methods of administering to a human or animal a pharmaceutical composition corresponding to any of the pharmaceutical compositions described in any of the above Embodiments.

### CLOSING PARAGRAPH—GLOBAL INCORPORATION BY REFERENCE

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A carrier composition comprising:
one or more surfactants, and
one or more neutral lipids,
wherein the carrier composition increases the bioavailability of one or more poorly water-soluble pharmaceutical agents that enhance intercellular interactions between cells possessing integrins and cell possessing corresponding integrin ligands when administered to an animal or human.

2. The composition of claim 1, further comprising:
one or more organic solvents.

3. The composition of claim 2, wherein the carrier composition comprises:
between about 99 wt. % and about 75 wt. % of a surfactant/neutral lipid mixture of the one or more surfactants and the one or more neutral lipids or any subrange thereof, and between about 1 wt. % and about 25 wt. % of the one or more organic solvents or any subrange thereof.

4. The composition of claim 3, wherein the surfactant/neutral lipid mixture comprises:
between about 5 wt. % and about 95 wt. % of the one or more surfactants or any subrange thereof, and
between about 95 wt. % and about 5 wt. % of the one or more neutral lipids or any subrange thereof.

5. The composition of claim 4, wherein:
the one or more organic solvents comprises:
one or more alcohols,
one or more non-alcohol polar solvents, or
any mixture thereof;
the one or more surfactants comprise:
one or more zwitterionic surfactants (ZISs),
one or more nonionic surfactants (NISs), or
any combination thereof;
the one or more neutral lipids comprise:
one or more short chain monoglycerides (SCMs),
one or more middle chain monoglycerides (MCMs),
one or more long chain monoglycerides (LCMs),
one or more long chain monoglycerides (VLCMs),
one or more short chain diglycerides (SCDs),
one or more middle chain diglycerides (MCDs),
one or more long chain diglycerides (LCDs),
one or more long chain monoglycerides (VLCDs),
one or more short chain triglycerides (SCTs),
one or more middle chain triglycerides (MCTs),
one or more long chain triglycerides (LCTs),
one or more long chain monoglycerides (VLCTs),
one or more short-chain fatty acid esters (SCFE),
one or more medium-chain fatty acid esters (MCFE),
one or more long-chain fatty acid esters (LCFE),
one or more very long chain fatty acid esters (VLCFE), or
any mixture thereof.

6. The composition of claim 5, wherein the one or more surfactants comprise:
one or more ZISs and any subrange thereof, and
one or more NISs and any subrange thereof.

7. The composition of claim 6, wherein the one or more ZISs comprising:
one or more phospholipids (PLs).

8. The composition of claim 6, wherein the one or more surfactants further comprising:
one or more ionic surfactants (ISs).

9. The composition of claim 8, wherein the one or more surfactants comprise:
between about 1 wt. % and about 10 wt. % of the one or more ISs or any subrange thereof, and
between about 99 wt. % to about 90 wt. % of the one or more ZISs and the one or more NISs or any subrange thereof.

10. The composition of claim 1, further comprising one or more other adjuvants or excipients.

11. A pharmaceutical composition comprising:
a carrier composition comprising:
one or more surfactants, and
one or more neutral lipids, and
one or more poorly water-soluble pharmaceutical agents,
wherein the carrier composition increases the bioavailability of the one or more poorly water-soluble pharmaceutical agents and the one or more poorly water-soluble pharmaceutical agents enhance intercellular interaction between cells possessing integrins and cell possessing corresponding integrin ligands when administered to an animal or human.

12. The composition of claim 11, further comprising:
one or more organic solvents.

13. The composition of claim 12, wherein the carrier composition comprises:
between about 99 wt. % and about 75 wt. % of a surfactant/neutral lipid mixture of the one or more surfactants and the one or more neutral lipids or any subrange thereof, and
between about 1 wt. % and about 25 wt. % of the one or more organic solvents or any subrange thereof.

14. The composition of claim 13, wherein the surfactant/neutral lipid mixture comprises:
between about 5 wt. % and about 95 wt. % of the one or more surfactants or any subrange thereof, and
between about 95 wt. % and about 5 wt. % of the one or more neutral lipids or any subrange thereof.

15. The composition of claim 14, wherein:
the one or more organic solvents comprises:
one or more alcohols,
one or more non-alcohol polar solvents, or
any mixture thereof;
the one or more surfactants comprise:
one or more zwitterionic surfactants (ZISs),
one or more nonionic surfactants (NISs), or
any combination thereof;
the one or more neutral lipids comprise:
one or more short chain monoglycerides (SCMs),
one or more middle chain monoglycerides (MCMs),
one or more long chain monoglycerides (LCMs),
one or more long chain monoglycerides (VLCMs),
one or more short chain diglycerides (SCDs),
one or more middle chain diglycerides (MCDs),
one or more long chain diglycerides (LCDs),
one or more long chain monoglycerides (VLCDs),
one or more short chain triglycerides (SCTs),
one or more middle chain triglycerides (MCTs),
one or more long chain triglycerides (LCTs),
one or more long chain monoglycerides (VLCTs),
one or more short-chain fatty acid esters (SCFE),
one or more medium-chain fatty acid esters (MCFE),
one or more long-chain fatty acid esters (LCFE),
one or more very long chain fatty acid esters (VLCFE), or
any mixture thereof.

16. The composition of claim 15, wherein the one or more surfactants comprise:
one or more ZISs and any subrange thereof, and
one or more NISs and any subrange thereof.

17. The composition of claim 16, wherein the one or more ZISs comprising:
one or more phospholipids (PLs).

18. The composition of claim 16, wherein the one or more surfactants further comprising:
one or more ionic surfactants (ISs).

19. The composition of claim 18, wherein the one or more surfactants comprise:
between about 1 wt. % and about 10 wt. % of the one or more ISs or any subrange thereof, and
between about 99 wt. % to about 90 wt. % of the one or more ZISs and the one or more NISs or any subrange thereof.

20. The composition of claim 11, further comprising one or more other adjuvants or excipients.

21. The composition of claim 11, wherein the one or more poorly water-soluble pharmaceutical agents comprising one or more poorly water-soluble, integrin agonist adjuvant pharmaceutical agents having a Log P value in n-octanol v. water of greater than 5 (Log P>5).

22. The composition of claim 21, wherein:
the one or more poorly water-soluble, integrin agonist adjuvant pharmaceutical agents comprising:
one or more compounds of general Formula (I),
one or more compounds of Formula (II), or
any mixture thereof;
the integrins include α4β1, α4β7, α5β1, αLβ2 and/or αVβ3;
the ligands include VCAM-1, fibronectin, MAdCAM-1, ICAM-1, ICAM-2, and/or vitronectin.
